(19) United States
(12) Patent Application Publication
(10) Pub. No.: US 2017/0369945 A1
(43) Pub. Date: Dec. 28, 2017

(54) METHODS OF DIAGNOSING AUTISM SPECTRUM DISORDERS

(71) Applicant: The Board of Trustees of the Leland Stanford Junior University, Stanford, CA (US)

(72) Inventors: Jingjing Li, Mountain View, CA (US); Minyi Shi, Palo Alto, CA (US); Michael Snyder, Stanford, CA (US)

(21) Appl. No.: 15/540,644

(22) PCT Filed: Dec. 28, 2015
(86) PCT No.: PCT/US15/67712

§ 371 Date: Jun. 29, 2017

Related U.S. Application Data
(60) Provisional application No. 62/097,568, filed on Dec. 29, 2014.

(51) Int. Cl. C12Q 1/68; G09B 19/00

(52) U.S. Cl. CPC C12Q 1/6883; G09B 19/00; C12Q 2600/156; C12Q 2600/172

(57) ABSTRACT

Methods of screening subjects for genetic markers associated with autism spectrum disorders are disclosed. In particular, the invention relates to methods of diagnosing autism spectrum disorders by detecting the presence of deleterious mutations or aberrant expression of genes associated with autism spectrum disorders. The present invention relates to genetic markers associated with ASD and methods of screening subjects for such genetic markers. In particular, the invention relates to methods of diagnosing ASD by detecting the presence of deleterious mutations or aberrant expression of genes associated with ASD.

### STATEMENT REGARDING FEDERALLY SPONSORED RESEARCH OR DEVELOPMENT

This invention was made with Government support under contract HG007735 awarded by the National Institutes of Health. The Government has certain rights in the invention.

### TECHNICAL FIELD

The present invention pertains to genetic markers of autism spectrum disorders (ASD) and methods of screening subjects for such genetic markers for diagnosis of ASD.

### BACKGROUND

Genetic studies of ASD in the past decade have implicated a large number of clinical mutations in more than 300 different human genes (Basu et al. (2009) Nucleic Acids Res 37:D832-D836). These mutations account for very few autism cases, suggesting that the genetic architecture of autism is comprised of extreme locus heterogeneity (Abrahams & Geschwind (2008) Nat Rev Genet 9:341-355). Key issues in understanding the underlying pathophysiology of ASDs are identifying and characterizing the shared molecular pathways perturbed by the diverse set of ASD mutations (Berg & Geschwind (2012) Genome Biol 13:247; Bill & Geschwind (2009) Curr Opin Genet Dev 19:271-278).

The common approach to uncover pathways underlying ASD is based on enrichment tests against a set of annotated pathways for mutations derived from a genome-wide comparison between cases and controls. For example, a β-catenin/chromatin remodeling protein network showed enrichment for the de novo mutations identified from sequencing exomes of sporadic cases with autism (O'Roak et al. (2012) Nature 485:246-250). Common variants from genome-wide association studies (GWAS) were also tested against KEGG pathways, suggesting a possible association with a pathway for ketone body metabolism (Yaspan et al. (2011) Hum Genet 129:563-571). However, in spite of extensive efforts by many research groups worldwide, including recent large-scale genotyping and sequencing studies (Anney et al. (2012) Hum Mol Genet 21:4781-4792; Liu et al. (2013) PLoS Genet 9:e1003443), we still lack a complete understanding of the genetic underpinnings of this disease.

There remains a need for identifying genetic markers associated with ASD and better methods of screening subjects for ASD.

### SUMMARY OF THE INVENTION

The present invention relates to genetic markers associated with ASD and methods of screening subjects for such genetic markers. In particular, the invention relates to methods of diagnosing ASD by detecting the presence of deleterious mutations or aberrant expression of genes associated with ASD.

Genetic markers associated with ASD that can be used in the practice of the invention include any gene, including, but not limited to, GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprising a mutation associated with ASD or that exhibits aberrant expression associated with ASD. Genetic markers associated with ASD may comprise deleterious mutations, for example, that perturb gene regulation or impair gene function. Such mutations may comprise a substitution, an insertion, a deletion, or a rearrangement. In certain embodiments, the mutation is a missense mutation, a nonsense mutation, a frameshift mutation, a splice-site mutation, a single nucleotide polymorphism, an inversion, or a translocation. In addition, ASD may also be associated with copy number variation of a gene. These genetic markers can be used alone or in combination with one or more additional genetic markers or relevant clinical parameters in prognosis, diagnosis, or monitoring treatment of ASD.

In one aspect, the invention includes a method of screening a subject for genetic markers associated with ASD. The method comprises: a) collecting a biological sample from the subject; and b) analyzing the biological sample to determine whether a gene selected from the group consisting of GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprises a mutation associated with ASD.

In one embodiment, the method further comprises determining which allele is present at a single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894, wherein the presence of a mutation at the single nucleotide polymorphism indicates that the subject has ASD.

In another embodiment, the method further comprises determining which allele is present at a single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266, wherein the presence of a mutation at the single nucleotide polymorphism indicates that the subject has ASD.

In certain embodiments, the method comprises analyzing the biological sample for multiple genetic markers described herein. In one embodiment, the method comprises analyzing the biological sample to determine whether the genes GRIN2B, SHANK2, TBR1, DLGAP2, SYNGAP1, LRP2, NLGN3, SHANK3, and ERBB2IP comprise a mutation associated with ASD. In another embodiment, the method comprises analyzing the biological sample to determine whether the genes ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprise a mutation associated with ASD. In another embodiment, the method comprises analyzing the biological sample to determine whether the genes GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprise a mutation associated with ASD. In another embodiment, the method comprises analyzing the biological sample to determine whether the genes ACTN4, ANKS1B, BCAS1, DGKZ, DLG1, DLGAP1, ERBB2IP, EXOC3, EXOC5, EXOC6, GDA, GRID2IP, GRIK3, IL16, KCNJ12, KCNJ15, KIF13B, LPL, LRP2BP, MAPK12, MPP6, MYOZ1, NOS1, SCN5A, THAP8, TNN, and UTRN comprise a mutation associated with ASD. In another embodiment, the method comprises analyzing the biological sample to determine whether the genes ANKS1B, DLG1, ERBB2IP, GRID2IP, GRIK3, KCNJ12, KCNJ15, NOS1, SCN5A, and UTRN comprise a mutation associated with ASD. In yet another embodiment, the method comprises analyzing the biological sample to determine whether the genes CNTNAP4, DLG4, DMD, GRIK2, INPP1, KIF13B, KCNJ10, LRP2, NLGN3, NLGN4X, SCN1A, and SHANK2 comprise a mutation associated with ASD.

In certain embodiments, a subject is screened for copy number variation of at least one gene selected from the group consisting of CAMK2B, DLG1, DLG4, DLGAP2, DLGAP3, DLGAP4, DYNLL1, EXOC3, KCND2, MAPK12, NLGN2, NLGN3, NLGN4X, NOS1, SHANK2, SNTA1, and SYNGAP1, wherein detection of copy number variation of at least one gene indicates that the subject has ASD. In one embodiment, the subject is screened for copy number variation of the genes SHANK2, DLGAP2, and SYNGAP1 genes, wherein detection of copy number variation of at least one gene indicates that the subject has ASD. Screening for copy number variation may be performed separately or in combination with screening for mutations.

The biological sample obtained from a subject for genetic testing is typically blood, serum, plasma, saliva, or cells from buccal swabbing, but can be any sample from bodily fluids, tissue or cells that contains genomic DNA or RNA of the subject. For prenatal testing of a fetus, the biological sample can be, for example, amniotic fluid (e.g., amniocentesis), placental tissue (e.g., chorionic villus sampling), or fetal blood (e.g., umbilical cord blood sampling). In certain embodiments, nucleic acids from the biological sample are further isolated, purified, and/or amplified prior to analysis.

The presence of a particular mutation associated with ASD in the genotype of the subject at can be determined by a variety of methods including, but not limited to, hybridization-based methods using allele-specific probes, such as dynamic allele-specific hybridization (DASH), microarray analysis, detection with molecular beacons, and SNP microarray analysis; PCR-based methods, such as Tetra-primer ARMS-PCR and the TaqMan 5′-nuclease assay; enzyme-based methods, such as the Invader assay with Flap endonuclease (FEN), the Serial Invasive Signal Amplification Reaction (SISAR), the oligonucleotide ligase assay, and restriction fragment length polymorphism (RFLP); and various other methods, such as single-strand conformation polymorphism, temperature gradient gel electrophoresis (TGGE), denaturing high performance liquid chromatography (DHPLC), sequencing, and immunoassay.

The subject may be any individual suspected of having ASD or a genetic predisposition for developing ASD. For example, the subject may be a developmentally disabled child, the parent of a developmentally disabled child, or have a sibling, parent, or other relative who has ASD. Early behavior training may be provided for any child diagnosed with ASD according to a method described herein.

In another aspect, the invention includes a method of determining risk of a human offspring developing ASD, the method comprising detecting in a biological sample from the mother or potential mother of the offspring at least one mutation associated with ASD in a gene selected from the group consisting of GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN, wherein the presence of at least one mutation indicates an increased risk of the offspring developing ASD. The offspring may be, for example, a neonate or a fetus. The biological sample may be obtained prior to or after conception. In one embodiment, the mother or potential mother has a previous child with ASD or a familial history of ASD.

In certain embodiments, the method comprises analyzing the biological sample for multiple genetic markers described herein. In one embodiment, the method comprises analyzing the biological sample from the mother to determine whether the genes GRIN2B, SHANK2, TBR1, DLGAP2, SYNGAP1, LRP2, NLGN3, SHANK3, and ERBB2IP comprise a mutation associated with ASD. In another embodiment, the method comprises analyzing the biological sample from the mother to determine whether the genes ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprise a mutation associated with ASD. In another embodiment, the method comprises analyzing the biological sample from the mother to determine whether the genes GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprise a mutation associated with ASD. In a further embodiment, the method comprises analyzing the biological sample from the mother to determine whether the genes ACTN4, ANKS1B, BCAS1, DGKZ, DLG1, DLGAP1, ERBB2IP, EXOC3, EXOC5, EXOC6, GDA, GRID2IP, GRIK3, IL16, KCNJ12, KCNJ15, KIF13B, LPL, LRP2BP, MAPK12, MPP6, MYOZ1, NOS1, SCN5A, THAP8, TNN, and UTRN comprise a mutation associated with ASD. In yet another embodiment, the method comprises analyzing the biological sample from the mother to determine whether the genes CNTNAP4, DLG4, DMD, GRIK2, INPP1, KIF13B, KCNJ10, LRP2, NLGN3, NLGN4X, SCN1A, and SHANK2 comprise a mutation associated with ASD.

In certain embodiments, the biological sample from the mother or potential mother may be screened for copy number variation of at least one gene selected from the group consisting of CAMK2B, DLG1, DLG4, DLGAP2, DLGAP3, DLGAP4, DYNLL1, EXOC3, KCND2, MAPK12, NLGN2, NLGN3, NLGN4X, NOS1, SHANK2, SNTA1, and SYNGAP1, wherein detection of copy number variation of at least one gene indicates that the offspring has ASD. In one embodiment, the biological sample is screened for copy number variation of the genes SHANK2, DLGAP2, and SYNGAP1 genes, wherein detection of copy number variation of at least one gene indicates that the offspring has ASD. Screening for copy number variation may be performed separately or in combination with screening for mutations.

In another aspect, the invention includes a kit for screening a subject for one or more genetic markers associated with ASD. The kit may include at least one agent for detecting one or more genetic markers associated with ASD (e.g., allele-specific hybridization probes, PCR primers, or SNP microarray), a container for holding a biological sample isolated from a human subject for genetic testing, and printed instructions for reacting the agent with the biological sample or a portion of the biological sample to determine whether or not a genetic marker associated with ASD is present in the biological sample. The agents may be packaged in separate containers. The kit may further comprise one or more control reference samples or other reagents for detecting genetic markers associated with ASD and genotyping a subject suspected of having ASD.

In one embodiment, the kit comprises at least one agent for analyzing one or more genes selected from the group consisting of GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN for determining the presence or absence of at least one mutation associated with ASD.

In another embodiment, the kit comprises at least one agent for determining which allele is present at a single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894, wherein the presence of a mutation at the single nucleotide polymorphism indicates that the subject has ASD.

In another embodiment, the kit comprises at least one agent for determining which allele is present at a single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266, wherein the presence of a mutation at the single nucleotide polymorphism indicates that the subject has ASD.

In another embodiment, the kit comprises at least one agent for determining whether the genes GRIN2B, SHANK2, TBR1, DLGAP2, SYNGAP1, LRP2, NLGN3, SHANK3, and ERBB2IP comprise a mutation associated with ASD.

In another embodiment, the kit comprises at least one agent for determining whether a gene selected from the group consisting of ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprises a mutation associated with ASD.

In another embodiment, the kit comprises at least one agent for determining whether a gene selected from the group consisting of ACTN4, ANKS1B, BCAS1, DGKZ, DLG1, DLGAP1, ERBB2IP, EXOC3, EXOC5, EXOC6, GDA, GRID2IP, GRIK3, IL16, KCNJ12, KCNJ15, KIF13B, LPL, LRP2BP, MAPK12, MPP6, MYOZ1, NOS1, SCN5A, THAP8, TNN, and UTRN comprises a mutation associated with ASD.

In another embodiment, the kit comprises at least one agent for determining whether a gene selected from the group consisting of CNTNAP4, DLG4, DMD, GRIK2, INPP1, KIF13B, KCNJ10, LRP2, NLGN3, NLGN4X, SCN1A, and SHANK2 comprises a mutation associated with ASD.

In another embodiment, the kit comprises at least one agent for analyzing a biological sample to detect copy number variation of at least one gene selected from the group consisting of CAMK2B, DLG1, DLG4, DLGAP2, DLGAP3, DLGAP4, DYNLL1, EXOC3, KCND2, MAPK12, NLGN2, NLGN3, NLGN4X, NOS1, SHANK2, SNTA1, and SYNGAP1.

In another embodiment, the kit comprises agents for analyzing a biological sample to detect copy number variation of the genes SHANK2, DLGAP2, and SYNGAP1.

In another aspect, the invention includes a method for diagnosing ASD in a subject, the method comprising: a) measuring the level of one or more biomarkers in a biological sample derived from the subject; and b) analyzing the levels of the one or more biomarkers in conjunction with respective reference value ranges for said one or more biomarkers, wherein differential expression of one or more biomarkers in the biological sample compared to one or more biomarkers in a control sample from a normal subject indicates that the subject has ASD.

Biomarkers that can be used in the practice of the invention include polynucleotides comprising nucleotide sequences from genes or RNA transcripts of genes, including but not limited to, ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCND2, KCNJ4, LDB3, LPL, NRXN2, PGM5, PTPRN, S100A3, SCN1A, SHANK2, SHANK3, TBR1, TJAP1, and ZDHHC23; or gene products thereof (e.g., proteins or peptides).

The reference value ranges can represent the levels of one or more biomarkers found in one or more samples of one or more subjects without ASD (e.g., normal, healthy subject). Alternatively, the reference values can represent the levels of one or more biomarkers found in one or more samples of one or more subjects with ASD. In certain embodiments, the levels of the biomarkers are compared to age-matched reference value ranges for normal subjects.

Biomarker polynucleotides (e.g., coding transcripts) can be detected, for example, by microarray analysis, polymerase chain reaction (PCR), reverse transcriptase (RT-PCR), Northern blot, or serial analysis of gene expression (SAGE).

Biomarker polypeptides can be measured, for example, by performing an enzyme-linked immunosorbent assay (ELISA), a radioimmunoassay (RIA), an immunofluorescent assay (IFA), immunohistochemistry (IHC), a sandwich assay, magnetic capture, microsphere capture, a Western Blot, surface enhanced Raman spectroscopy (SERS), flow cytometry, or mass spectrometry. In certain embodiments, the level of a biomarker is measured by contacting an antibody with the biomarker, wherein the antibody specifically binds to the biomarker, or a fragment thereof containing an antigenic determinant of the biomarker. Antibodies that can be used in the practice of the invention include, but are not limited to, monoclonal antibodies, polyclonal antibodies, chimeric antibodies, recombinant fragments of antibodies, Fab fragments, Fab′ fragments, F(ab′)₂ fragments, Fᵥ fragments, or scFᵥ fragments.

In certain embodiments, a panel of biomarkers is used for diagnosis of ASD. Biomarker panels of any size can be used in the practice of the invention. Biomarker panels for diagnosing ASD typically comprise at least 3 biomarkers and up to 30 biomarkers, including any number of biomarkers in between, such as 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the invention includes a biomarker panel comprising at least 3, at least 4, or at least 5, or at least 6, or at least 7, or at least 8, or at least 9, or at least 10, or at least 11 or more biomarkers. Although smaller biomarker panels are usually more economical, larger biomarker panels (i.e., greater than 30 biomarkers) have the advantage of providing more detailed information and can also be used in the practice of the invention.

In one embodiment, the invention includes a biomarker panel comprising a plurality of biomarkers for diagnosing ASD, wherein one or more biomarkers are selected from the group consisting of ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, and ZDHHC23.

In another aspect, the invention includes a kit for diagnosing ASD in a subject. The kit may include a container for holding a biological sample isolated from a human subject suspected of having ASD, at least one agent that specifically detects an ASD biomarker; and printed instructions for reacting the agent with the biological sample or a portion of the biological sample to detect the presence or amount of at least one ASD biomarker in the biological sample. The agents may be packaged in separate containers. The kit may further comprise one or more control reference samples and reagents for performing an immunoassay and/or PCR, and/or microarray analysis for detection of biomarkers as described herein.

In another aspect, the invention includes a composition comprising at least one in vitro complex comprising a labeled probe hybridized to a nucleic acid comprising a biomarker ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, or ZDHHC23 gene sequence, said labeled probe hybridized to said biomarker gene sequence, or complement thereof, wherein said nucleic acid is extracted from a patient who has an ASD, or is an amplification product of a nucleic acid extracted from a patient who has the ASD. Probes may be detectably labeled with any type of label, including, but not limited to, a fluorescent label, bioluminescent label, chemiluminescent label, colorimetric label, or isotopic label (e.g., stable trace isotope or radioactive isotope). In certain embodiments, the composition is in a detection device (i.e., device capable of detecting labeled probe).

In certain embodiments, the composition comprises a plurality of in vitro complexes, wherein each nucleic acid comprising a biomarker ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, or ZDHHC23 gene sequence, or complement thereof, is hybridized to a complementary labeled probe.

In another aspect, the invention includes a set of primers or probes for diagnosing a subject with ASD comprising a plurality of primers or probes for detecting a plurality of target nucleic acids, wherein the plurality of target nucleic acids comprises one or more gene sequences, or complements thereof, of genes selected from the group consisting of ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, and ZDHHC23. Primers and probes may be detectably labeled with any type of label, including, but not limited to, a fluorescent label, bioluminescent label, chemiluminescent label, colorimetric label, or isotopic label (e.g., stable trace isotope or radioactive isotope).

In certain embodiments, the set of primers or probes is capable of detecting a plurality of target nucleic acids collectively comprising the gene sequences, or complements thereof, of the genes ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, and ZDHHC23.

In another aspect, the invention includes a composition comprising at least one in vitro complex comprising a labeled allele-specific probe hybridized to a nucleic acid comprising a GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, or UTRN gene sequence, said labeled allele-specific probe hybridized to said gene sequence, or complement thereof, wherein said nucleic acid is extracted from a patient who has an autism spectrum disorder (ASD), or is an amplification product of a nucleic acid extracted from a patient who has ASD. Allele-specific probes may be detectably labeled with any type of label, including, but not limited to, a fluorescent label, bioluminescent label, chemiluminescent label, colorimetric label, or isotopic label (e.g., stable trace isotope or radioactive isotope). In certain embodiments, the composition is in a detection device (i.e., device capable of detecting labeled probe).

In certain embodiments, the composition comprises a plurality of in vitro complexes, wherein each nucleic acid comprising a GRIN2B, SHANK2, TBR1, DLGAP2, SYNGAP1, LRP2, NLGN3, SHANK3, or ERBB2IP gene sequence, or complement thereof, is hybridized to a complementary labeled probe.

In other embodiments, the composition comprises at least one in vitro complex comprising a nucleic acid comprising a single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894 hybridized to a labeled allele-specific probe.

In other embodiments, the composition comprises at least one in vitro complex comprising a nucleic acid comprising at least one single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266 hybridized to a labeled allele-specific probe.

In another aspect, the invention includes a set of primers or probes for diagnosing a subject with ASD comprising a plurality of allele-specific primers or allele-specific probes for detecting a plurality of target nucleic acids, wherein the plurality of target nucleic acids comprises one or more gene sequences, or complements thereof, of genes selected from the group consisting of GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN. Allele-specific primers or allele-specific probes may be detectably labeled with any type of label, including, but not limited to, a fluorescent label, bioluminescent label, chemiluminescent label, colorimetric label, or isotopic label (e.g., stable trace isotope or radioactive isotope).

In certain embodiments, the set of allele-specific primers or allele-specific probes is capable of detecting a plurality of target nucleic acids collectively comprising the gene sequences, or complements thereof, of the genes GRIN2B, SHANK2, TBR1, DLGAP2, SYNGAP1, LRP2, NLGN3, SHANK3, and ERBB2IP.

In other embodiments, the set of allele-specific primers or allele-specific probes is capable of detecting at least one single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894. In one embodiment, the set of allele-specific primers or allele-specific probes is capable of detecting the single nucleotide polymorphisms rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894.

In other embodiments, the set of allele-specific primers or allele-specific probes is capable of detecting at least one single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266. In one embodiment, the set of allele-specific primers or allele-specific probes is capable of detecting single nucleotide polymorphism at the chromosome positions chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266.

These and other embodiments of the subject invention will readily occur to those of skill in the art in view of the disclosure herein.

### BRIEF DESCRIPTION OF THE FIGURES

FIGS. 1A and 1B show candidate genes from sequencing screens. FIG. 1A shows an overview of the identified loci from whole-genome and exome sequencing. Evolutionary conservation is quantified by GERP++ score, where the higher scores indicate greater selective pressure on the genomic loci. For genes with multiple significant loci, the most conserved residue is considered. Variants absent in the 1000 Genome dataset are considered rare variants. The genes were colorized based on the fraction of deleterious mutations predicted by MutationTaster among all the identified mutations in the gene from this study. FIG. 1B shows replication using another larger patient cohort with >500 patients sequenced with the SOLiD platform. In this dataset, variants with allele frequencies with increased absolute differences between cases and controls are more likely to affect genes that were also detected in our study (light gray line). The allele frequency difference is the absolute differences between cases and controls. This trend cannot be observed by 10,000 simulations (dark gray line for one randomized dataset).

FIGS. 2A-2C show expression analysis of the synaptic module. FIG. 2A shows dichotomized expression of the genes in module #13 across 295 brain sections. Relative abundance of each gene across the 295 brain sections was hierarchically clustered to reveal gene groups exhibiting similar expression patterns across tissues. Group 1 genes showed elevated expression in 175 regions (T1, e.g. corpus callosum) relative to other brain sections, and Group 2 genes showed high expression in 120 regions (e.g. hippocampal regions) (T2) relative to other brain sections. FIG. 2B shows RNA-sequencing of 4 different brain regions from a healthy subject. The brain regions include the Brodmann areas 9 (BA9), 40 (BA40), the amygdala (AMY) and the corpus callosum (CC), which revealed the same observation as the microarray analyses. Group 1 (light gray) and 2 (dark gray) genes were compared with 1000 randomly sampled genes (medium grey) from the transcriptome in each brain region. The raw FPKM values were normalized into the cumulative density functions based on kernel density estimation. The elevation of Group 2 genes across all brain regions and the greatest increase of Group 1 genes in the corpus callosum were all statistically significant (P<1e-5, Wilcoxon ranksum test). FIG. 2C shows RNA-sequencing of the corpus callosum transcriptomes from 6 non-autistic individuals. FPKM quantifies the absolute expression of genes in each group. The two groups have similar expression in the corpus callosum (P>0.5, Wilcoxon ranksum test), which, however, are all above the transcriptome background (P<4.87e-6, Wilcoxon ranksum test), suggesting that both sub-components are active in this tissue.

FIGS. 3A-3E show cell-type expression of this module in oligodendrocytes. FIG. 3A shows immunohistochemisry analysis in the corpus callosum. Staining of LRP2 in the human corpus callosum reveals that the major cell population in the corpus callosum is oligodendrocytes (the round nuclei) which express LRP2 stained in brown. A zoom-in view is shown in the inset. FIG. 3B shows neural cell-type expression of the orthologous module #13 in mouse brain. Gene expression in different neural cell types was hierarchically clustered into the 3 major cell types in brain (neurons, oligodendrocytes and astrocytes), which also grouped genes in this module into a neuron cluster and a glial cluster, enriched for Group 1 and 2 genes (panel A), respectively. The fraction of Group 1 (light gray) and 2 (dark gray) genes in the neuron and glial clusters were represented by the pie charts, with statistical significance determined by a chi-square test. FIG. 3C shows overall expression of the module in cultured oligodendrocyte precursor cells (OPCs). Group 1 and 2 were expressed at the similar level with the transcriptome background. FIG. 3D shows the role of the module in oligodendrocyte (OL) development. Differentiation of OPCs into mature myelinating OLs (MOG+) led to a significant up-regulation of Group 1 genes (left, OPCs->mature OLs). On the other hand, conditional knockout (CKO) of the master myelination factor MRF from mature OLs led to a significant up-regulation of Group 2 genes (right, mature OLs->MRF CKO). FIG. 3E shows a proposed model. Up-regulation is associated with, or likely to contribute to, the differentiation of OPCs into mature myelinating OLs. The mature OLs acquire their myelination capacity by activating the MRF-mediated regulatory network, which also serves to repress expression of Group 2 genes.

FIGS. 4A-4D show an integrative analysis of the genetic alteration in this study. FIG. 4A shows enrichment of the differentially expressed genes in this ASD module. RNA-sequencing corpus callosum of autism patients and their matched controls. Enrichment was not observed for the genes in the human synaptome or the collection of known autism genes. FIG. 4B shows the mutation pattern of the genes from the innermost layers of the interaction network (K≧10) to the periphery layer (K=1). Genes in the central and periphery layers in this module are more likely to be affected, while the trend cannot be observed in 10,000 random simulations. For individual bins, a significant enrichment and depletion was observed in the central layers (K≧10) and the intermediate layers (3≦K<6), respectively. FIG. 4C shows compositional bias of the mutated genes in central layers. The mutated genes in central layers are more biased towards the corpus-callosum specific subcomponent; this trend is not observed in background or other mutated genes with varying degree of K. FIG. 4D shows a positive correlation between network coreness and gene expression in corpus callosum. RNA-sequencing of 6 non-autistic individual's corpus callosum reveal the positive correlation, suggesting the central layers may play critical roles in corpus callosum. Two outlier genes DYNLL1 and BCAS1 are separately labeled due to their extreme expression in this tissue.

FIG. 5 shows a flowchart of the study design. This study first uncovered an ASD-related module, followed by validation among ASD patients, and by functional characterization using network and transcriptome analyses. In the Discovery panel, the red nodes are genes previously known to be associated with ASD. In the Integrative analysis panel, blue and red nodes represent excessive mutation and differential expression for a given gene in the network.

FIG. 6 shows co-expression of the interacting proteins. A comparison among randomly sampled protein pairs (random on the x-axis), HINT (a recently benchmarked protein interaction network) and BioGrid revealed that interacting proteins in BioGrid have the highest expression correlation among 79 human tissues and cell types.

FIGS. 7A and 7B show topological properties of the network modules. FIG. 7A shows that the cluster size distribution follows a power-law. The inset of the histogram is a log-log plot for the cluster sizes showing a significant scale-free property. FIG. 7B shows the elevated network modularity Q for the real human protein interaction network. A set of 100 randomized networks was generated to determine the statistical significance. The random simulation preserved the number of interacting partners for each node but randomly rewired the interactions.

FIG. 8A shows FDR distribution of GO enrichment for the protein modules. The vast majority of the modules are highly significant with FDR<5e-3. FIG. 8B shows the threshold selection to determine the size of the clusters showing GO enrichment. The number of enriched clusters plotted against this threshold varied from 1 to 20 (the dark gray line with circles). The gradient of the line at each threshold was shown in black (with squares). We chose to use n=5 as a threshold in our analysis, which represents a transition point from a rapid increase of the gradient towards full convergence.

FIG. 9 shows hierarchical clustering of the enrichment map for the topological modules. Gray pixels indicate GO term enrichment (arranged along the horizontal axis) for each module (arranged along the vertical axis). Exemplar terms are also highlighted in the map. The right panel depicts the enrichment (false discovery rates, FDRs) of each module for a collection of known autism genes and generic human disease genes. Insignificant FDR is set to 1, and the two autism-associated modules are enriched for transcriptional regulation and neuron synaptic transmission, respectively. 3 different modules showed enrichment for the genes involved in generic human disease genes.

FIG. 10 shows enrichment for the ASD genes in this module #13. The enrichment tests were performed on the known SFARI ASD genes from different releases. The newly added genes are those from September 2012 to July 2013, representing the growth of our knowledge.

FIG. 11 shows absolute expression of genes in the 2 groups across the 295 brain sections. The median of each group in each tissue (in black) was compared with the transcriptome median (shared by both groups, in light gray). The zoom-in view shows an elevation of gene expression of Group 1 in the corpus callosum, where Group 2 genes are a down-regulated, which are all above the transcriptome background (in light gray).

FIGS. 12A and 12B show expression propensity for genes in the 2 groups. Increased expression specificity index for genes in Group 1 (FIG. 12A) is consistent with its reduced expression breadth (FIG. 12B). Expression breadth is defined to be the number of the tissues where a gene is expressed. 3 cutoffs were used to determine the absence/presence of a gene in a tissue, representing the 5%, 25% and the 50% percentiles of the expression data across all genes and all tissues.

FIG. 13 shows the biased expression of LRP2 in the corpus callosum. RNA-sequencing for the Brodmann areas 9 (BA9), 40 (BA40), and the amygdala (AMY) was from a typically developing individual. LRP2 expression in CC was evaluated based on its expression across the six control subjects in our study.

FIGS. 14A-14D show immunohistochemistry analysis of LRP2 in the human corpus callosum. FIG. 14A shows a control subject, who was also immunostained with anti-LRP2, whose specificity was determined by a positive control and two sets of negative control (FIGS. 14B-14D). FIG. 14B shows the positive control is the staining in human kidney carcinoma, which is known to have an extremely high LRP2 protein level. FIG. 14C shows IgG staining in the corpus callosum, which was used as the first negative control. FIG. 14D shows LRP2 staining in the normal human ovary, which was used as the second negative control, where the absence of LRP2 has been indicated in literature.

FIG. 15 shows a Pearson's correlation between 2 biological replicates of RNA-seq experiments. Biological replicates were performed on 6 samples, where different sections from the tissue blocks were assayed. Genes with extreme expression (FPKM>50, accounting for less than 1%) were excluded from the analysis.

FIG. 16 shows the layered structure of the protein interaction network in this study. K-core decomposition was used to partition the network. The visualization was implemented by LaNet-vi. Node colors follow the rainbow color scale with violet for the most peripheral nodes (K=1), and red nodes with the greatest K in the network.

FIG. 17 shows the cumulative distribution of the node coreness in the network. In this analysis, we considered nodes in the network center with K≧10, where >80% of proteins in the network were below this threshold.

### DETAILED DESCRIPTION OF THE INVENTION

The practice of the present invention will employ, unless otherwise indicated, conventional methods of genetics, chemistry, biochemistry, molecular biology and recombinant DNA techniques, within the skill of the art. Such techniques are explained fully in the literature. See, e.g., Autism Spectrum Disorders (D. Amaral, D. Geschwind, and G. Dawson eds., Oxford University Press, 2011); Autism Spectrum Disorders: Identification, Education, and Treatment (D. Zager, D. Cihak, A. K. Stone-MacDonald eds., Routledge; 3rd edition, 2004); Single Nucleotide Polymorphisms: Methods and Protocols (Methods in Molecular Biology, A. A. Komar ed., Humana Press; 2^{nd} edition, 2009); Genetic Variation: Methods and Protocols (Methods in Molecular Biology, M. R. Barnes and G. Breen eds., Humana Press, 2010); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a polynucleotide” includes a mixture of two or more such polynucleotides, and the like.

As used herein, the term “autism spectrum disorder” refers to a neurodevelopmental disorder including, but not limited to autism, Asperger syndrome, pervasive developmental disorder not otherwise specified (PDD-NOS), childhood disintegrative disorder, and Rett syndrome.

The term “genetic marker associated with ASD” refers to a gene or gene product (e.g., RNA transcript or protein) that distinguishes subjects who have ASD from control subjects (e.g., a person with a negative diagnosis, normal or healthy subject). Genetic markers include genetic variations associated with development of ASD, such as genes (or gene products thereof) comprising deleterious mutations, such as those perturbing gene regulation or impairing gene function, or copy number variation resulting in abnormal levels of a gene product.

The terms “polymorphism,” “polymorphic nucleotide,” “polymorphic site” or “polymorphic nucleotide position” refer to a position in a nucleic acid that possesses the quality or character of occurring in several different forms. A nucleic acid may be naturally or non-naturally polymorphic, e.g., having one or more sequence differences (e.g., additions, deletions and/or substitutions) as compared to a reference sequence. A reference sequence may be based on publicly available information (e.g., the U.C. Santa Cruz Human Genome Browser Gateway (genome.ucsc.edu/cgi-bin/hgGateway) or the NCBI website (ncbi.nlm.nih.gov)) or may be determined by a practitioner of the present invention using methods well known in the art (e.g., by sequencing a reference nucleic acid). A nucleic acid polymorphism is characterized by two or more “alleles”, or versions of the nucleic acid sequence. Typically, an allele of a polymorphism that is identical to a reference sequence is referred to as a “reference allele” and an allele of a polymorphism that is different from a reference sequence is referred to as an “alternate allele,” or sometimes a “variant allele”. As used herein, the term “major allele” refers to the more frequently occurring allele at a given polymorphic site, and “minor allele” refers to the less frequently occurring allele, as present in the general or study population.

The term “single nucleotide polymorphism” or “SNP” refers to a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. Single nucleotide polymorphisms can also arise from a deletion of a nucleotide or an insertion of a nucleotide relative to a reference allele.

The terms “hybridize” and “hybridization” refer to the formation of complexes between nucleotide sequences which are sufficiently complementary to form complexes via Watson Crick base pairing. The terms are intended to refer to the formation of a specific hybrid between a probe and a target region.

The term “derived from” is used herein to identify the original source of a molecule but is not meant to limit the method by which the molecule is made which can be, for example, by chemical synthesis or recombinant means.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

“Substantially purified” generally refers to isolation of a substance (compound, polynucleotide, oligonucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides oligonucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide or oligonucleotide is a nucleic acid molecule devoid, in whole or part, of sequences normally associated with it in nature; or a sequence, as it exists in nature, but having heterologous sequences in association therewith; or a molecule disassociated from the chromosome.

The terms “polypeptide” and “protein” refer to a polymer of amino acid residues and are not limited to a minimum length. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54:3607-3630; Obika et al. (1998) Tetrahedron Lett. 39: 5401-5404.

As used herein, the term “probe” or “oligonucleotide probe” refers to a polynucleotide, as defined above, that contains a nucleic acid sequence complementary to a nucleic acid sequence present in the target nucleic acid analyte (e.g., at SNP location). The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally.

An “allele-specific probe” hybridizes to only one of the possible alleles of a SNP under suitably stringent hybridization conditions.

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, nucleic acids are amplified using at least one set of oligonucleotide primers comprising at least one forward primer and at least one reverse primer capable of hybridizing to regions of a nucleic acid flanking the portion of the nucleic acid to be amplified.

An “allele-specific primer” matches the sequence exactly of only one of the possible alleles of a SNP, hybridizes at the SNP location, and amplifies only one specific allele if it is present in a nucleic acid amplification reaction.

The term “amplicon” refers to the amplified nucleic acid product of a PCR reaction or other nucleic acid amplification process (e.g., ligase chain reaction (LGR), nucleic acid sequence based amplification (NASBA), transcription-mediated amplification (TMA), Q-beta amplification, strand displacement amplification, or target mediated amplification). Amplicons may comprise RNA or DNA depending on the technique used for amplification. For example, DNA amplicons may be generated by RT-PCR, whereas RNA amplicons may be generated by TMA/NASBA.

The terms “subject,” “individual,” and “patient,” are used interchangeably herein and refer to any mammalian subject, particularly humans. Other subjects may include cattle, dogs, cats, guinea pigs, rabbits, rats, mice, horses, and so on. In some cases, the methods of the invention find use in experimental animals, in veterinary application, and in the development of animal models, including, but not limited to, rodents including mice, rats, and hamsters; and primates.

A “biomarker” in the context of the present invention refers to a biological compound, such as a polypeptide or polynucleotide which is differentially expressed in a sample taken from patients having ASD as compared to a comparable sample taken from control subjects (e.g., a person with a negative diagnosis, normal or healthy subject). The biomarker can be a nucleic acid, a fragment of a nucleic acid, a polynucleotide, or an oligonucleotide or a protein, a fragment of a protein, a peptide, or a polypeptide that can be detected and/or quantified. ASD biomarkers include polynucleotides comprising nucleotide sequences from genes or RNA transcripts of genes, including but not limited to, ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, and ZDHHC23; or gene products thereof (e.g., proteins or peptides).

The phrase “differentially expressed” refers to differences in the quantity and/or the frequency of a biomarker present in a sample taken from patients having, for example, ASD as compared to a control subject. For example, a biomarker can be a polypeptide or polynucleotide which is present at an elevated level or at a decreased level in samples of patients with ASD compared to samples of control subjects. Alternatively, a biomarker can be a polypeptide or polynucleotide which is detected at a higher frequency or at a lower frequency in samples of patients with ASD compared to samples of control subjects. A biomarker can be differentially present in terms of quantity, frequency or both.

A polypeptide or polynucleotide is differentially expressed between two samples if the amount of the polypeptide or polynucleotide in one sample is statistically significantly different from the amount of the polypeptide or polynucleotide in the other sample. For example, a polypeptide or polynucleotide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide or polynucleotide is differentially expressed in two sets of samples if the frequency of detecting the polypeptide or polynucleotide in samples of patients' suffering from ASD, is statistically significantly higher or lower than in the control samples. For example, a polypeptide or polynucleotide is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

A “similarity value” is a number that represents the degree of similarity between two things being compared. For example, a similarity value may be a number that indicates the overall similarity between a patient's expression profile using specific phenotype-related biomarkers and reference value ranges for the biomarkers in one or more control samples or a reference expression profile (e.g., the similarity to an “ASD” expression profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

As used herein, a “biological sample” refers to a sample of tissue, cells, or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents, including but not limited to, conditioned media resulting from the growth of cells and tissues in culture medium, e.g., recombinant cells, and cell components. For prenatal testing of a fetus, the biological sample can be, for example, amniotic fluid (e.g., amniocentesis), placental tissue (e.g., chorionic villus sampling), or fetal blood (e.g., umbilical cord blood sampling).

A “test amount” of a biomarker refers to an amount of a biomarker present in a sample being tested. A test amount can be either an absolute amount (e.g., μg/ml) or a relative amount (e.g., relative intensity of signals).

A “diagnostic amount” of a biomarker refers to an amount of a biomarker in a subject's sample that is consistent with a diagnosis of ASD. A diagnostic amount can be either an absolute amount (e.g., μg/ml) or a relative amount (e.g., relative intensity of signals).

A “control amount” of a biomarker can be any amount or a range of amount which is to be compared against a test amount of a biomarker. For example, a control amount of a biomarker can be the amount of a biomarker in a person without ASD. A control amount can be either in absolute amount (e.g., μg/ml) or a relative amount (e.g., relative intensity of signals).

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Immunoassay” is an assay that uses an antibody to specifically bind an antigen (e.g., a biomarker). The immunoassay is characterized by the use of specific binding properties of a particular antibody to isolate, target, and/or quantify the antigen. An immunoassay for a biomarker may utilize one antibody or several antibodies. Immunoassay protocols may be based, for example, upon competition, direct reaction, or sandwich type assays using, for example, labeled antibody. The labels may be, for example, fluorescent, chemiluminescent, or radioactive.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a biomarker from specific species such as rat, mouse, or human can be selected to obtain only those polyclonal antibodies that are specifically immunoreactive with the biomarker and not with other proteins, except for polymorphic variants and alleles of the biomarker. This selection may be achieved by subtracting out antibodies that cross-react with biomarker molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Capture reagent” refers to a molecule or group of molecules that specifically bind to a specific target molecule or group of target molecules. For example, a capture reagent can comprise two or more antibodies each antibody having specificity for a separate target molecule. Capture reagents can be any combination of organic or inorganic chemicals, or biomolecules, and all fragments, analogs, homologs, conjugates, and derivatives thereof that can specifically bind a target molecule.

The capture reagent can comprise a single molecule that can form a complex with multiple targets, for example, a multimeric fusion protein with multiple binding sites for different targets. The capture reagent can comprise multiple molecules each having specificity for a different target, thereby resulting in multiple capture reagent-target complexes. In certain embodiments, the capture reagent is comprised of proteins, such as antibodies.

The capture reagent can be directly labeled with a detectable moiety. For example, an anti-biomarker antibody can be directly conjugated to a detectable moiety and used in the inventive methods, devices, and kits. In the alternative, detection of the capture reagent-biomarker complex can be by a secondary reagent that specifically binds to the biomarker or the capture reagent-biomarker complex. The secondary reagent can be any biomolecule, and is preferably an antibody. The secondary reagent is labeled with a detectable moiety. In some embodiments, the capture reagent or secondary reagent is coupled to biotin, and contacted with avidin or streptavidin having a detectable moiety tag.

“Detectable moieties” or “detectable labels” contemplated for use in the invention include, but are not limited to, radioisotopes, fluorescent dyes such as fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), Cyan Fluorescent Protein (CFP), Yellow Fluorescent Protein (YFP), Cerianthus Orange Fluorescent Protein (cOFP), alkaline phosphatase (AP), β-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), lacZ (encoding α-galactosidase), and xanthine guanine phosphoribosyltransferase (XGPRT), β-Glucuronidase (gus), Placental Alkaline Phosphatase (PLAP), Secreted Embryonic Alkaline Phosphatase (SEAP), or Firefly or Bacterial Luciferase (LUC). Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of genetic markers that are especially useful in diagnosing ASD (see Example 1). In particular, the inventors have used interactome, gene, and genome sequencing to identify genetic markers associated with ASD. Sequencing of 25 patients confirmed the involvement of certain genes in autism, which was subsequently validated using an independent cohort of over 500 patients. RNA-sequencing of the corpus callosum from patients with autism showed that these genes exhibited extensive misexpression.

In order to further an understanding of the invention, a more detailed discussion is provided below regarding the identified genetic markers associated with ASD and methods of screening subjects for such genetic markers for diagnosing ASD.

A. Detecting Genetic Markers Associated with ASD

In one aspect, the invention provides methods of diagnosing ASD by detecting the presence of deleterious mutations associated with ASD in genes, including GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN, which can be used singly or in combination as genetic markers for determining whether a subject is likely to have ASD.

Mutations associated with ASD, for example, may impair gene regulation or gene function. A large number of clinical mutations have been identified in subjects with ASD and can be used as genetic markers of ASD. Representative mutations are presented in Example 1 and additional representative mutations are listed in the SFARI Gene database (gene.sfari.org/autdb/). See, for example, SFARI entries: GEN111, GEN229, GEN477, GEN066, GEN245, GEN362, GEN171, GEN230, GEN616, GEN412, GEN070, GEN109, GEN135, GEN140, GEN362, GEN171, GEN172, and GEN223; all of which entries (as entered by the date of filing of this application) are herein incorporated by reference. In addition, mutations associated with ASD are also described in O'Roak et al. (2011) Nat Genet. 43(6):585-589; O'Roak et al. (2012) Science 338(6114):1619-1622; Talkowski et al. (2012) Cell 149(3):525-537; Awadalla et al. (2010) Am J Hum Genet. 87(3):316-324; Klassen et al. (2011) Cell 145(7):1036-1048; de Ligt et al. (2012) N Engl J Med. 367(20):1921-1929; Tarabeux et al. (2011) Transl. Psychiatry 1:e55; Dimassi et al. (2013) Am J Med Genet A. 161A(10):2564-2569; Epi4K Consortium et al. (2013) Nature 501(7466):217-221; Endele et al. (2010) Nat. Genet. 42(11):1021-1026; Freunscht et al. (2013) Behav Brain Funct. 9:20; Kenny et al. (2014) Mol Psychiatry 19(8):872-879; Lemke et al. (2014) Ann Neurol. 75(1):147-154; De Rubeis et al. (2014) Nature 515(7526):209-215; Berkel et al. (2010) Nat Genet. 42(6):489-491; Liu et al. (2013) PLoS One 8(2):e56639; Leblond et al. (2012) PLoS Genet. 8(2):e1002521; Pinto et al. (2010) Nature 466(7304):368-372; Chilian et al. (2013) Clin Genet. 84(6):560-565; Schluth-Bolard et al. (2013) J Med Genet. 50(3):144-150; Schluth-Bolard et al. (2013) J Med Genet. 50(3):144-150; Prasad et al. (2012) G3 (Bethesda) 2(12):1665-1685; Rauch et al. (2012) Lancet. 380(9854):1674-1682; Sanders et al. (2012) Nature 485(7397):237-241; Hwang et al. (2005) J Biol Chem. 280(13):12467-12473; Sheng et al. (2000) J Cell Sci. 113 (Pt 11):1851-1856; Leblond et al. (2014) PLoS Genet. 10(9):e1004580; Berkel et al. (2012) Hum Mol Genet. 21(2):344-357; O'Roak et al. (2012) Nature 485(7397):246-250; Traylor et al. (2012) Mol Syndromol. 3(3):102-112; Neale et al. (2012) Nature 485(7397):242-245; Palumbo et al. (2014) Am J Med Genet A. 164A(3):828-833; De Rubeis et al. (2014) Nature. 515(7526):209-215; Deriziotis et al. (2014) Nat Commun. 5:4954; Marshall et al. (2008) Am J Hum Genet. 82(2):477-488; Cukier et al. (2014) Mol Autism. 5(1):1; Chien et al. (2013) Mol Autism 4(1):26; Pinto et al. (2010) Nature 466(7304):368-372; Hamdan et al. (2011) Biol Psychiatry 69(9):898-901; Writzl et al. (2013)Am J Med Genet A. 161A(7):1682-1685; Brett et al. (2014) PLoS One 9(4):e93409; Hamdan et al. (2011) Am J Hum Genet. 88(3):306-316; Krepischi et al. (2010) Am J Med Genet A. 152A(9):2376-2378; Carvill et al. (2013) Nat Genet. 45(7):825-830; Berryer et al. (2013) Hum Mutat. 34(2):385-394; Ionita-Laza et al. (2012) Am J Hum Genet. 90(6):1002-1013; Iossifov et al. (2012) Neuron 74(2):285-299; Kantarci et al. (2007) Nat Genet. 39(8):957-959; Jamain et al. (2003) Nat Genet. 34(1):27-29; Yu et al. (2011) Behav Brain Funct. 7:13; Ylisaukko-oja et al. (2005) Eur J Hum Genet. 13(12):1285-1292; Steinberg et al. (2012) Mol Autism. 3(1):8; Yanagi et al. (2012) Autism Res Treat. 2012:724072; Jiang et al. (2013) Am J Hum Genet. 93(2):249-263; Yu et al. (2013) Neuron 77(2):259-273; Jaramillo et al. (2014) Autism Res. 7(2):264-272; De Jaco et al. (2006) J Biol Chem. 281(14):9667-9676; Földy et al. (2013) Neuron. 78(3):498-450; Rothwell et al. (2014) Cell 158(1):198-212; Durand et al. (2007) Nat Genet. 39(1):25-27; Kolevzon et al. (2011) Brain Res. 1380:98-105; Qin et al. (2009) BMC Med Genet. 10:61; Sykes et al. (2009) Eur J Hum Genet. 17(10):1347-1353; Waga et al. (2011) Psychiatr Genet. 21(4):208-211; Coe et al. (2014) Nat Genet. 46(10):1063-1071; Gauthier et al. (2009) Am J Med Genet B Neuropsychiatr Genet. 150B(3):421-424; Koshimizu et al. (2013) PLoS One. 8(9):e74167; Moessner et al. (2007) Am J Hum Genet. 81(6):1289-1297; Kelleher et al. (2012) PLoS One 7(4):e35003; Leblond et al. (2014) PLoS Genet. 10(9):e1004580; Boccuto et al. (2013) Eur J Hum Genet. 21(3):310-31; Zhu et al. (2014) Hum Mol Genet. 23(6):1563-1578; herein incorporated by reference in their entireties. Any of the described mutations associated with ASD may be used as a genetic marker of ASD as described herein.

In one embodiment, the method further comprises determining which allele is present at a single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894, wherein the presence of a mutation at the single nucleotide polymorphism indicates that the subject has ASD.

In another embodiment, the method further comprises determining which allele is present at a single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266, wherein the presence of a mutation at the single nucleotide polymorphism indicates that the subject has ASD.

In certain embodiments, the method comprises analyzing the biological sample for multiple genetic markers described herein. In one embodiment, the method comprises analyzing the biological sample to determine whether the genes GRIN2B, SHANK2, TBR1, DLGAP2, SYNGAP1, LRP2, NLGN3, SHANK3, and ERBB2IP comprise a mutation associated with ASD.

In another embodiment, the method comprises analyzing the biological sample to determine whether the genes ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprise a mutation associated with ASD.

In another embodiment, the method comprises analyzing the biological sample to determine whether the genes GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprise a mutation associated with ASD.

In another embodiment, the method comprises analyzing the biological sample to determine whether the genes ACTN4, ANKS1B, BCAS1, DGKZ, DLG1, DLGAP1, ERBB2IP, EXOC3, EXOC5, EXOC6, GDA, GRID2IP, GRIK3, IL16, KCNJ12, KCNJ15, KIF13B, LPL, LRP2BP, MAPK12, MPP6, MYOZ1, NOS1, SCN5A, THAP8, TNN, and UTRN comprise a mutation associated with ASD.

In another embodiment, the method comprises analyzing the biological sample to determine whether the genes ANKS1B, DLG1, ERBB2IP, GRID2IP, GRIK3, KCNJ12, KCNJ15, NOS1, SCN5A, and UTRN comprise a mutation associated with ASD.

In yet another embodiment, the method comprises analyzing the biological sample to determine whether the genes CNTNAP4, DLG4, DMD, GRIK2, INPP1, KIF13B, KCNJ10, LRP2, NLGN3, NLGN4X, SCN1A, and SHANK2 comprise a mutation associated with ASD.

In addition, the methods of the invention can be used to assess the risk of a human offspring developing ASD. A biological sample can be collected from the mother or potential mother of an offspring prior to conception or after conception and analyzed for one or more genetic markers association with ASD. Detection of at least one genetic marker associated with ASD, as described herein, indicates an increased risk of the offspring developing ASD. The offspring may be, for example, a neonate or a fetus. In particular, this method can be used to evaluate a mother or potential mother potentially at high risk of having a child with ASD, such as a mother or potential mother who has had a previous child with ASD or a familial history of ASD.

For genetic testing, a biological sample containing nucleic acids is collected from an individual suspected of having ASD. The biological sample is typically blood, saliva, or cells from buccal swabbing, but can be any sample from bodily fluids, tissue, or cells that contains genomic DNA or RNA of the individual. For prenatal testing of a fetus, the biological sample can be, for example, amniotic fluid (e.g., amniocentesis), placental tissue (e.g., chorionic villus sampling), or fetal blood (e.g., umbilical cord blood sampling). In certain embodiments, nucleic acids from the biological sample are isolated, purified, and/or amplified prior to analysis using methods well-known in the art. See, e.g., Green and Sambrook Molecular Cloning: A Laboratory Manual (Cold Spring Harbor Laboratory Press; 4^{th} edition, 2012); and Current Protocols in Molecular Biology (Ausubel ed., John Wiley & Sons, 1995); herein incorporated by reference in their entireties.

It is understood that genetic markers associated with ASD can be detected in a sample by any suitable method known in the art. Detection of a nucleic acid comprising a mutation associated with ASD can be direct or indirect. For example, the nucleic acid itself can be detected directly. Alternatively, a genetic marker can be detected indirectly from cDNAs, amplified RNAs or DNAs, or proteins expressed by a gene comprising a mutation associated with ASD. Any method that detects a single base change in a nucleic acid sample can be used. For example, allele-specific probes that specifically hybridize to a nucleic acid containing a mutation associated with ASD can be used to detect the genetic marker. A variety of nucleic acid hybridization formats are known to those skilled in the art. For example, common formats include sandwich assays and competition or displacement assays. Hybridization techniques are generally described in Hames, and Higgins “Nucleic Acid Hybridization, A Practical Approach,” IRL Press (1985); Gall and Pardue, Proc. Natl. Acad. Sci. U.S.A., 63:378-383 (1969); and John et al Nature, 223:582-587 (1969).

Sandwich assays are commercially useful hybridization assays for detecting or isolating nucleic acids. Such assays utilize a “capture” nucleic acid covalently immobilized to a solid support and a labeled “signal” nucleic acid in solution. The clinical sample will provide the target nucleic acid. The “capture” nucleic acid and “signal” nucleic acid probe hybridize with the target nucleic acid to form a “sandwich” hybridization complex.

In one embodiment, the allele-specific probe is a molecular beacon. Molecular beacons are hairpin shaped oligonucleotides with an internally quenched fluorophore. Molecular beacons typically comprise four parts: a loop of about 18-30 nucleotides, which is complementary to the target nucleic acid sequence; a stem formed by two oligonucleotide regions that are complementary to each other, each about 5 to 7 nucleotide residues in length, on either side of the loop; a fluorophore covalently attached to the 5′ end of the molecular beacon, and a quencher covalently attached to the 3′ end of the molecular beacon. When the beacon is in its closed hairpin conformation, the quencher resides in proximity to the fluorophore, which results in quenching of the fluorescent emission from the fluorophore. In the presence of a target nucleic acid having a region that is complementary to the strand in the molecular beacon loop, hybridization occurs resulting in the formation of a duplex between the target nucleic acid and the molecular beacon. Hybridization disrupts intramolecular interactions in the stem of the molecular beacon and causes the fluorophore and the quencher of the molecular beacon to separate resulting in a fluorescent signal from the fluorophore that indicates the presence of the target nucleic acid sequence.

For detection of a genetic marker, the molecular beacon is designed to only emit fluorescence when bound to a specific allele. When the molecular beacon probe encounters a target sequence with as little as one non-complementary nucleotide, the molecular beacon preferentially stay in its natural hairpin state and no fluorescence is observed because the fluorophore remains quenched. See, e.g., Nguyen et al. (2011) Chemistry 17(46):13052-13058; Sato et al. (2011) Chemistry 17(41):11650-11656; Li et al. (2011) Biosens Bioelectron. 26(5):2317-2322; Guo et al. (2012) Anal. Bioanal. Chem. 402(10):3115-3125; Wang et al. (2009) Angew. Chem. Int. Ed. Engl. 48(5):856-870; and Li et al. (2008) Biochem. Biophys. Res. Commun. 373(4):457-461; herein incorporated by reference in their entireties.

Probes can readily be synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109. Poly(A) or poly(C), or other non-complementary nucleotide extensions may be incorporated into polynucleotides using these same methods. Hexaethylene oxide extensions may be coupled to the polynucleotides by methods known in the art. Cload et al., J. Am. Chem. Soc. (1991) 113:6324-6326; U.S. Pat. No. 4,914,210 to Levenson et al.; Durand et al., Nucleic Acids Res. (1990) 18:6353-6359; and Horn et al., Tet. Lett. (1986) 27:4705-4708.

Alternatively, probes can be produced by amplification of a target nucleic acid using, e.g., polymerase chain reaction (PCR), nucleic acid sequence based amplification (NASBA), ligase chain reaction (LCR), self-sustained sequence replication (3 SR), Q-beta amplification; strand displacement amplification, or any other nucleic acid amplification method to produce a probe capable of hybridizing to the desired target sequence.

The probes may be coupled to labels for detection. There are several means known for derivatizing polynucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al., Nucl. Acids Res. (1985) 13:1529-1541 which discloses biotinylation of the 5′ termini of polynucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly, Nucl. Acids Res. (1987) 15:3131-3139, Gibson et al. Nucl. Acids Res. (1987) 15:6455-6467 and U.S. Pat. No. 4,605,735 to Miyoshi et al. Methods are also available for synthesizing sulfhydryl-derivatized polynucleotides, which can be reacted with thiol-specific labels, see, e.g., U.S. Pat. No. 4,757,141 to Fung et al., Connolly et al., Nucl. Acids Res. (1985) 13:4485-4502 and Spoat et al. Nucl. Acids Res. (1987) 15:4837-4848. A comprehensive review of methodologies for labeling DNA fragments is provided in Matthews et al., Anal. Biochem. (1988) 169:1-25.

For example, probes may be fluorescently labeled. Guidance for selecting appropriate fluorescent labels can be found in Smith et al., Meth. Enzymol. (1987) 155:260-301; Karger et al., Nucl. Acids Res. (1991) 19:4955-4962; Guo et al. (2012) Anal. Bioanal. Chem. 402(10):3115-3125; and Molecular Probes Handbook, A Guide to Fluorescent Probes and Labeling Technologies, 11th edition, Johnson and Spence eds., 2010 (Molecular Probes/Life Technologies); herein incorporated by reference. Fluorescent labels include fluorescein and derivatives thereof, such as disclosed in U.S. Pat. No. 4,318,846 and Lee et al., Cytometry (1989) 10:151-164. Dyes for use in the present invention include 3-phenyl-7-isocyanatocoumarin, methyl coumarin-3-acetic acid (AMCA), acridines, such as 9-isothiocyanatoacridine and acridine orange, pyrenes, benzoxadiazoles, and stilbenes, such as disclosed in U.S. Pat. No.4,174,384. Additional dyes include SYBR green, SYBR gold, Yakima Yellow, Texas Red, 3-(ε-carboxypentyl)-3′-ethyl-5,5′-dimethyloxa-carbocyanine (CYA); 6-carboxy fluorescein (FAM); CAL Fluor Orange 560, CAL Fluor Red 610, Quasar Blue 670; 5,6-carboxyrhodamine-110 (R110); 6-carboxyrhodamine-6G (R6G); N′,N′,N′,N′-tetramethyl-6-carboxyrhodamine (TAMRA); 6-carboxy-X-rhodamine (ROX); 2′, 4′, 5′, 7′, -tetrachloro-4-7-dichlorofluorescein (TET); 2′, 7′-dimethoxy-4′, 5′-6 carboxyrhodamine (JOE); 6-carboxy-2′,4,4′,5′,7,7′-hexachlorofluorescein (HEX); Dragonfly orange; ATTO-Tec; Bodipy; ALEXA; VIC, Cy3, CyS, and Cy7. These dyes are commercially available from various suppliers such as Life Technologies (Carlsbad, Calif.), Biosearch Technologies (Novato, Calif.), and Integrated DNA Technolgies (Coralville, Iowa). Fluorescent labels include fluorescein and derivatives thereof, such as disclosed in U.S. Pat. No. 4,318,846 and Lee et al., Cytometry (1989) 10:151-164, and 6-FAM, JOE, TAMRA, ROX, HEX-1, HEX-2, ZOE, TET-1 or NAN-2, and the like.

Fluorophores may be covalently attached to a particular nucleotide, for example, and the labeled nucleotide incorporated into the probe using standard techniques such as nick translation, random priming, and PCR labeling. Alternatively, a fluorophore may be covalently attached via a linker to a deoxycytidine nucleotide that has been transaminated. Methods for labeling probes are described in U.S. Pat. No. 5,491,224 and Molecular Cytogenetics: Protocols and Applications (2002), Y.-S. Fan, Ed., Chapter 2, “Labeling Fluorescence In Situ Hybridization Probes for Genomic Targets,” L. Morrison et al., p. 21-40, Humana Press; which are herein incorporated by reference.

One of skill in the art will recognize that other luminescent agents or dyes may be used in lieu of fluorophores as label containing moieties. Other luminescent agents, which may be used, include, for example, radioluminescent, chemiluminescent, bioluminescent, and phosphorescent label containing moieties, as well as quantum dots. Alternatively, in situ hybridization of chromosomal probes may be employed with the use of detection moieties visualized by indirect means. Probes may be labeled with biotin or digoxygenin using routine methods known in the art, and then further processed for detection. Visualization of a biotin-containing probe may be achieved via subsequent binding of avidin conjugated to a detectable marker. Chromosomal probes hybridized to target regions may alternatively be visualized by enzymatic reactions of label moieties with suitable substrates for the production of insoluble color products. Each probe may be discriminated from other probes within the set by choice of a distinct label. A biotin-containing probe within a set may be detected via subsequent incubation with avidin conjugated to alkaline phosphatase (AP) or horseradish peroxidase (HRP) and a suitable substrate, e.g., 5-bromo-4-chloro-3-indolylphosphate and nitro blue tetrazolium (NBT) serve as substrates for alkaline phosphatase, whereas diaminobenzidine serves as a substrate for HRP.

In another embodiment, detection of a genetic marker sequence is performed using allele specific amplification. In the case of PCR, amplification primers can be designed to bind to a portion of one of the disclosed genes, and the terminal base at the 3′ end is used to discriminate between the major and minor alleles or mutant and wild-type forms of the genes. If the terminal base matches the major or minor allele, polymerase-dependent three prime extension can proceed. Amplification products can be detected with specific probes. This method for detecting point mutations or polymorphisms is described in detail by Sommer et al. in Mayo Clin. Proc. 64:1361-1372 (1989).

Tetra-primer ARMS-PCR uses two pairs of primers that can amplify two alleles in one PCR reaction. Allele-specific primers are used that hybridize at the location of a genetic marker, but each matches perfectly to only one of the possible alleles. If a given allele is present in the PCR reaction, the primer pair specific to that allele will amplify that allele, but not another allele. The two primer pairs for the different alleles may be designed such that their PCR products are of significantly different length, which allows them to be distinguished readily by gel electrophoresis. See, e.g., Muñoz et al. (2009) J. Microbiol. Methods 78(2):245-246 and Chiapparino et al. (2004) Genome. 47(2):414-420; herein incorporated by reference.

Genetic markers may also be detected by ligase chain reaction (LCR) or ligase detection reaction (LDR). The specificity of the ligation reaction is used to discriminate between alleles at the site of the genetic marker. Two probes are hybridized at the polymorphic site of a nucleic acid of interest, whereby ligation can only occur if the probes are identical to the target sequence. See e.g., Psifidi et al. (2011) PLoS One 6(1):e14560; Asari et al. (2010) Mol. Cell. Probes 24(6):381-386; Lowe et al. (2010) Anal Chem. 82(13):5810-5814; herein incorporated by reference.

Genetic markers can also be detected in a biological sample by sequencing and genotyping. In the former method, one simply carries out whole genome sequencing of a patient sample, and uses the results to detect the sequences present. Whole genome analysis is used in the field of “personal genomics,” and genetic testing services exist, which provide full genome sequencing using massively parallel sequencing. Massively parallel sequencing is described e.g. in U.S. Pat. No. 5,695,934, entitled “Massively parallel sequencing of sorted polynucleotides,” and US 2010/0113283 A1, entitled “Massively multiplexed sequencing.” Massively parallel sequencing typically involves obtaining DNA representing an entire genome, fragmenting it, and obtaining millions of random short sequences, which are assembled by mapping them to a reference genome sequence.

Genetic analysis can be carried out by a variety of methods that do not involve massively parallel random sequencing. As described below, a commercially available MassARRAY system can be used. This system uses matrix-assisted laser desorption ionization time-of-flight mass spectrometry (MALDI-TOF MS) coupled with single-base extension PCR for high-throughput multiplex detection of genetic markers. Another commercial system is made by Illumina. The Illumina Golden Gate assay generates specific PCR products for genetic markers that are subsequently hybridized to beads either on a solid matrix or in solution. Three oligonucleotides are synthesized for each genetic marker: two allele specific oligos (ASOs) that distinguish the genetic marker, and a locus specific sequence (LSO) just downstream of the genetic marker. The ASO and LSO sequences also contain target sequences for a set of universal primers (P1 through P3 in the adjacent figure), while each LSO also contains a particular address sequences (the “illumicode”) complementary to sequences attached to beads.

As another example, Affymetrix SNP arrays use multiple sets of short oligonucleotide probes for known SNPs. The design of a SNP array such as manufactured by Affymetrix and Illumina is described further in LaFamboise, “Single nucleotide polymorphism arrays: a decade of biological, computational and technological advances,” Nuc. Acids Res. 37(13):4181-4193 (2009), which provides additional description of methods for detecting SNPs.

Another technology useful in analysis of genetic markers is PCR-dynamic allele specific hybridization (DASH), which involves dynamic heating and coincident monitoring of DNA denaturation, as disclosed by Howell et al. (Nat. Biotech. 17:87-88, 1999). A target sequence is amplified (e.g., by PCR) using one biotinylated primer. The biotinylated product strand is bound to a streptavidin-coated microtiter plate well (or other suitable surface), and the non-biotinylated strand is rinsed away with alkali wash solution. An oligonucleotide probe, specific for one allele (e.g., the wild-type allele), is hybridized to the target at low temperature. This probe forms a duplex DNA region that interacts with a double strand-specific intercalating dye. When subsequently excited, the dye emits fluorescence proportional to the amount of double-stranded DNA (probetarget duplex) present. The sample is then steadily heated while fluorescence is continually monitored. A rapid fall in fluorescence indicates the denaturing temperature of the probe-target duplex. Using this technique, a single-base mismatch between the probe and target results in a significant lowering of melting temperature (Tm) that can be readily detected.

A variety of other techniques can be used to detect genetic markers, including but not limited to, the Invader assay with Flap endonuclease (FEN), the Serial Invasive Signal Amplification Reaction (SISAR), the oligonucleotide ligase assay, restriction fragment length polymorphism (RFLP), single-strand conformation polymorphism, temperature gradient gel electrophoresis (TGGE), and denaturing high performance liquid chromatography (DHPLC). See, for example Molecular Analysis and Genome Discovery (R. Rapley and S. Harbron eds., Wiley 1^{st} edition, 2004); Jones et al. (2009) New Phytol. 183(4):935-966; Kwok et al. (2003) Curr Issues Mol. Biol. 5(2):43-60; Muñoz et al. (2009) J. Microbiol. Methods 78(2):245-246; Chiapparino et al. (2004) Genome. 47(2):414-420; Olivier (2005) Mutat Res. 573(1-2):103-110; Hsu et al. (2001) Clin. Chem. 47(8):1373-1377; Hall et al. (2000) Proc. Natl. Acad. Sci. U.S.A. 97(15):8272-8277; Li et al. (2011) J. Nanosci. Nanotechnol. 11(2):994-1003; Tang et al. (2009) Hum. Mutat. 30(10):1460-1468; Chuang et al. (2008) Anticancer Res. 28(4A):2001-2007; Chang et al. (2006) BMC Genomics 7:30; Galeano et al. (2009) BMC Genomics 10:629; Larsen et al. (2001) Pharmacogenomics 2(4):387-399; Yu et al. (2006) Curr. Protoc. Hum. Genet. Chapter 7: Unit 7.10; Lilleberg (2003) Curr. Opin. Drug Discov. Devel. 6(2):237-252; and U.S. Pat. Nos. 4,666,828; 4,801,531; 5,110,920; 5,268,267; 5,387,506; 5,691,153; 5,698,339; 5,736,330; 5,834,200; 5,922,542; and 5,998,137 for a description of such methods; herein incorporated by reference in their entireties.

If the genetic marker is located in the coding region of a gene of interest, the genetic marker can be identified indirectly by detection of the variant protein produced by the gene. Variant proteins (i.e., containing an amino acid substitution encoded by the allele comprising the genetic marker) can be detected using antibodies specific for the variant protein. For example, immunoassays that can be used to detect variant proteins produced by an allele comprising a genetic marker include, but are not limited to, immunohistochemistry (IHC), western blotting, enzyme-linked immunosorbent assay (ELISA), radioimmunoassays (RIA), “sandwich” immunoassays, fluorescent immunoassays, and immunoprecipitation assays, the procedures of which are well known in the art (see, e.g., Schwarz et al. (2010) Clin. Chem. Lab. Med. 48(12):1745-1749; The Immunoassay Handbook (D. G. Wild ed., Elsevier Science; 3^{rd} edition, 2005); Ausubel et al, eds, 1994, Current Protocols in Molecular Biology, Vol. 1 (John Wiley & Sons, Inc., New York); Coligan Current Protocols in Immunology (1991); Harlow & Lane, Antibodies: A Laboratory Manual (1988); Handbook of Experimental Immunology, Vols. I-IV (D. M. Weir and C. C. Blackwell eds., Blackwell Scientific Publications); herein incorporated by reference herein in their entireties).

In addition, copy number variation of certain genes is associated with ASD and may be used as a genetic marker. Copy number variation can be calculated based on “relative copy number” so that apparent differences in gene copy numbers in different samples are not distorted by differences in sample amounts. The relative copy number of a gene (per genome) can be expressed as the ratio of the copy number of a target gene to the copy number of a reference polynucleotide sequence in a DNA sample. The reference polynucleotide sequence can be a sequence having a known genomic copy number. Typically the reference sequence will have a single genomic copy and is a sequence that is not likely to be amplified or deleted in the genome. It is not necessary to empirically determine the copy number of a reference sequence. Rather, the copy number may be assumed based on the normal copy number in the organism of interest. Accordingly, the relative copy number of the target nucleotide sequence in a DNA sample is calculated from the ratio of the two genes. In certain embodiments, a subject is screened for copy number variation of at least one gene selected from the group consisting of CAMK2B, DLG1, DLG4, DLGAP2, DLGAP3, DLGAP4, DYNLL1, EXOC3, KCND2, MAPK12, NLGN2, NLGN3, NLGN4X, NOS1, SHANK2, SNTA1, and SYNGAP1, wherein detection of copy number variation, that is, the presence of a greater or fewer number of a gene (i.e., abnormal copy number) in the subject compared to a control subject (e.g., normal, healthy subject) indicates that the subject has ASD. In one embodiment, the subject is screened for copy number variation of the SHANK2, DLGAP2, and SYNGAP1 genes, wherein detection of copy number variation in at least one gene indicates that the subject has ASD. Screening for copy number variation may be performed separately or in combination with screening for mutations.

B. Kits for Screening for Genetic Markers

In yet another aspect, the invention provides kits for screening a subject for genetic markers associated with ASD. The kit may include one or more agents for detection of a nucleic acid comprising a mutation associated with ASD, such as allele-specific hybridization probes, PCR primers, or a microarray for determining which allele is present. The kit may further comprise a container for holding a biological sample isolated from a human subject for genetic testing and printed instructions for reacting agents with the biological sample or a portion of the biological sample to detect the presence of at least one genetic marker associated with ASD in the biological sample. The agents may be packaged in separate containers. The kit may further comprise one or more control reference samples or other reagents for detecting genetic markers associated with ASD and genotyping a subject suspected of having ASD.

In certain embodiments, the kit further comprises reagents for performing dynamic allele-specific hybridization (DASH), Tetra-primer ARMS-PCR, a TaqMan 5′-nuclease assay; an Invader assay with Flap endonuclease (FEN), a Serial Invasive Signal Amplification Reaction (SISAR), an oligonucleotide ligase assay, restriction fragment length polymorphism (RFLP), single-strand conformation polymorphism, temperature gradient gel electrophoresis (TGGE), denaturing high performance liquid chromatography (DHPLC), sequencing, or an immunoassay.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of detecting genetic markers associated with ASD and diagnosing ASD.

In certain embodiments, the kit comprises at least one agent for analyzing one or more genes selected from the group consisting of GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN for determining the presence or absence of at least one mutation associated with ASD.

In one embodiment, the kit comprises at least one agent for determining whether a gene selected from the group consisting of ACTN4, ANKS1B, BCAS1, DGKZ, DLG1, DLGAP1, ERBB2IP, EXOC3, EXOC5, EXOC6, GDA, GRID2IP, GRIK3, IL16, KCNJ12, KCNJ15, KIF13B, LPL, LRP2BP, MAPK12, MPP6, MYOZ1, NOS1, SCN5A, THAP8, TNN, and UTRN comprises a mutation associated with ASD.

In another embodiment, the kit comprises at least one agent for determining whether a gene selected from the group consisting of CNTNAP4, DLG4, DMD, GRIK2, INPP1, KIF13B, KCNJ10, LRP2, NLGN3, NLGN4X, SCN1A, and SHANK2 comprises a mutation associated with ASD.

In another embodiment, the kit comprises at least one agent for determining whether a gene selected from the group consisting of ANKS1B, DLG1, ERBB2IP, GRID2IP, GRIK3, KCNJ12, KCNJ15, NOS1, SCN5A, UTRN comprises a mutation associated with ASD.

In another embodiment, the kit comprises at least one agent for determining which allele is present at a single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894, wherein the presence of a mutation at the single nucleotide polymorphism indicates that the subject has ASD.

In another embodiment, the kit comprises at least one agent for determining which allele is present at a single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266, wherein the presence of a mutation at the single nucleotide polymorphism indicates that the subject has ASD.

In another embodiment, the kit comprises agents for analyzing a biological sample for multiple genetic markers described herein. In one embodiment, the kit comprises agents for determining whether the genes GRIN2B, SHANK2, TBR1, DLGAP2, SYNGAP1, LRP2, NLGN3, SHANK3, and ERBB2IP comprise a mutation associated with ASD. In another embodiment, the kit comprises agents for determining whether the genes ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprise a mutation associated with ASD. In another embodiment, the kit comprises agents for determining whether the genes GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprise a mutation associated with ASD. In another embodiment, the kit comprises agents for determining whether the genes ACTN4, ANKS1B, BCAS1, DGKZ, DLG1, DLGAP1, ERBB2IP, EXOC3, EXOC5, EXOC6, GDA, GRID2IP, GRIK3, IL16, KCNJ12, KCNJ15, KIF13B, LPL, LRP2BP, MAPK12, MPP6, MYOZ1, NOS1, SCN5A, THAP8, TNN, and UTRN comprise a mutation associated with ASD. In another embodiment, the kit comprises agents for determining whether the genes ANKS1B, DLG1, ERBB2IP, GRID2IP, GRIK3, KCNJ12, KCNJ15, NOS1, SCN5A, and UTRN comprise a mutation associated with ASD. In yet another embodiment, the kit comprises agents for determining whether the genes CNTNAP4, DLG4, DMD, GRIK2, INPP1, KIF13B, KCNJ10, LRP2, NLGN3, NLGN4X, SCN1A, and SHANK2 comprise a mutation associated with ASD.

In another embodiment, the kit comprises agents for analyzing a biological sample to detect copy number variation of at least one gene selected from the group consisting of CAMK2B, DLG1, DLG4, DLGAP2, DLGAP3, DLGAP4, DYNLL1, EXOC3, KCND2, MAPK12, NLGN2, NLGN3, NLGN4X, NOS1, SHANK2, SNTA1, and SYNGAP1.

In another embodiment, the kit comprises agents for analyzing a biological sample to detect copy number variation of the genes SHANK2, DLGAP2, and SYNGAP1.

C. Biomarkers Showing Differential Expression in Association with ASD

Biomarkers that can be used in the practice of the invention include polynucleotides comprising nucleotide sequences from genes or RNA transcripts of genes, including but not limited to, ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, and ZDHHC23; or gene products thereof (e.g., proteins or peptides). Differential expression of these biomarkers is associated with ASD and therefore expression profiles of these biomarkers are useful for diagnosing ASD.

Accordingly, in one aspect, the invention provides a method for diagnosing ASD in a subject, comprising measuring the levels of one or more biomarkers in a biological sample derived from a subject suspected of having ASD, and analyzing the levels of the biomarkers and comparing with respective reference value ranges for the biomarkers, wherein differential expression of one or more biomarkers in the biological sample compared to one or more biomarkers in a control sample indicates that the subject has ASD. When analyzing the levels of biomarkers in a biological sample, the reference value ranges used for comparison can represent the level of one or more biomarkers found in one or more samples of one or more subjects without ASD (i.e., normal or control samples). Alternatively, the reference values can represent the level of one or more biomarkers found in one or more samples of one or more subjects with ASD.

The biological sample obtained from the subject to be diagnosed can be any sample from bodily fluids, tissue or cells that contain the expressed biomarkers. A “control” sample, as used herein, refers to a biological sample, such as a bodily fluid, tissue, or cells that are not diseased. That is, a control sample is obtained from a normal subject (e.g. an individual known to not have ASD). A biological sample can be obtained from a subject by conventional techniques. For example, blood can be obtained by venipuncture, and solid tissue samples can be obtained by surgical techniques according to methods well known in the art.

In certain embodiments, a panel of biomarkers is used for diagnosis of ASD. Biomarker panels of any size can be used in the practice of the invention. Biomarker panels for diagnosing ASD typically comprise at least 3 biomarkers and up to 30 biomarkers, including any number of biomarkers in between, such as 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the invention includes a biomarker panel comprising at least 3, at least 4, or at least 5, or at least 6, or at least 7, or at least 8, or at least 9, or at least 10 or more biomarkers. Although smaller biomarker panels are usually more economical, larger biomarker panels (i.e., greater than 30 biomarkers) have the advantage of providing more detailed information and can also be used in the practice of the invention.

In certain embodiments, the invention includes a panel of biomarkers for diagnosing ASD comprising one or more polynucleotides comprising a nucleotide sequence from a gene or an RNA transcript of a gene selected from the group consisting of an ACTN2 polynucleotide, an ATP2B2 polynucleotide, a BCAS1 polynucleotide, a CAMK2A polynucleotide, a CNTNAP4 polynucleotide, a DGKZ polynucleotide, a DLGAP2 polynucleotide, a DLGAP3 polynucleotide, a DYNLL1 polynucleotide, a GDA polynucleotide, a GRIA1 polynucleotide, a GRIK3 polynucleotide, a GRIN2A polynucleotide, a GRIN2B polynucleotide, a HTR2C polynucleotide, a KCNA4 polynucleotide, a KCNJ2 polynucleotide, a KCNJ4 polynucleotide, a LDB3 polynucleotide, a LPL polynucleotide, a NRXN2 polynucleotide, a PGM5 polynucleotide, a PTPRN polynucleotide, a S100A3 polynucleotide, a SCN1A polynucleotide, a SHANK2 polynucleotide, a SHANK3 polynucleotide, a TBR1 polynucleotide, a TJAP1 polynucleotide, and a ZDHHC23 polynucleotide.

D. Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites) that are indicative of the expression level of the biomarker. The methods for measuring biomarkers in a sample have many applications. For example, one or more biomarkers can be measured to aid in the diagnosis of ASD, to determine the appropriate treatment for a subject, to monitor responses in a subject to treatment, or to identify therapeutic compounds that modulate expression of the biomarkers in vivo or in vitro.

Detecting Biomarker Polynucleotides

In one embodiment, the expression levels of the biomarkers are determined by measuring polynucleotide levels of the biomarkers. The levels of transcripts of specific biomarker genes can be determined from the amount of mRNA, or polynucleotides derived therefrom, present in a biological sample. Polynucleotides can be detected and quantitated by a variety of methods including, but not limited to, microarray analysis, polymerase chain reaction (PCR), reverse transcriptase polymerase chain reaction (RT-PCR), Northern blot, and serial analysis of gene expression (SAGE). See, e.g., Draghici Data Analysis Tools for DNA Microarrays, Chapman and Hall/CRC, 2003; Simon et al. Design and Analysis of DNA Microarray Investigations, Springer, 2004; Real-Time PCR: Current Technology and Applications, Logan, Edwards, and Saunders eds., Caister Academic Press, 2009; Bustin A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008; herein incorporated by reference in their entireties.

In one embodiment, microarrays are used to measure the levels of biomarkers. An advantage of microarray analysis is that the expression of each of the biomarkers can be measured simultaneously, and microarrays can be specifically designed to provide a diagnostic expression profile for a particular disease or condition (e.g., ASD). Biomarker polynucleotides which may be measured by microarray analysis can be expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)⁺ messenger RNA (mRNA) or a fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation (Chirgwin et al., 1979, Biochemistry 18:5294-5299), a silica gel-based column (e.g., RNeasy (Qiagen, Valencia, Calif.) or StrataPrep (Stratagene, La Jolla, Calif.)), or using phenol and chloroform, as described in Ausubel et al., eds., 1989, Current Protocols In Molecular Biology, Vol. III, Green Publishing Associates, Inc., John Wiley & Sons, Inc., New York, at pp. 13.12.1-13.12.5). Poly(A)⁺ RNA can be selected, e.g., by selection with oligo-dT cellulose or, alternatively, by oligo-dT primed reverse transcription of total cellular RNA. RNA can be fragmented by methods known in the art, e.g., by incubation with ZnCl₂, to generate fragments of RNA.

In one embodiment, total RNA, mRNA, or nucleic acids derived therefrom, are isolated from a sample taken from an ASD patient. Biomarker polynucleotides that are poorly expressed in particular cells may be enriched using normalization techniques (Bonaldo et al., 1996, Genome Res. 6:791-806).

In one embodiment, the invention includes a microarray comprising an oligonucleotide that hybridizes to a ACTN2 polynucleotide, an oligonucleotide that hybridizes to a ATP2B2 polynucleotide, an oligonucleotide that hybridizes to a BCAS1 polynucleotide, an oligonucleotide that hybridizes to a CAMK2A polynucleotide, an oligonucleotide that hybridizes to a CNTNAP4 polynucleotide, an oligonucleotide that hybridizes to a DGKZ polynucleotide, an oligonucleotide that hybridizes to a DLGAP2 polynucleotide, an oligonucleotide that hybridizes to a DLGAP3 polynucleotide, an oligonucleotide that hybridizes to a DYNLL1 polynucleotide, an oligonucleotide that hybridizes to a GDA polynucleotide, an oligonucleotide that hybridizes to a GRIA1 polynucleotide, an oligonucleotide that hybridizes to a GRIK3 polynucleotide, an oligonucleotide that hybridizes to a GRIN2A polynucleotide, an oligonucleotide that hybridizes to a GRIN2B polynucleotide, an oligonucleotide that hybridizes to a HTR2C polynucleotide, an oligonucleotide that hybridizes to a KCNA4 polynucleotide, an oligonucleotide that hybridizes to a KCNJ2 polynucleotide, an oligonucleotide that hybridizes to a KCNJ4 polynucleotide, an oligonucleotide that hybridizes to a LDB3 polynucleotide, an oligonucleotide that hybridizes to a LPL polynucleotide, an oligonucleotide that hybridizes to a NRXN2 polynucleotide, an oligonucleotide that hybridizes to a PGM5 polynucleotide, an oligonucleotide that hybridizes to a PTPRN polynucleotide, an oligonucleotide that hybridizes to a S100A3 polynucleotide, an oligonucleotide that hybridizes to a SCN1A polynucleotide, an oligonucleotide that hybridizes to a SHANK2 polynucleotide, an oligonucleotide that hybridizes to a SHANK3 polynucleotide, an oligonucleotide that hybridizes to a TBR1 polynucleotide, an oligonucleotide that hybridizes to a TJAP1 polynucleotide, and an oligonucleotide that hybridizes to a ZDHHC23 polynucleotide that can be used for detecting and measuring biomarker polynucleotides.

Polynucleotides can also be analyzed by other methods including, but not limited to, northern blotting, nuclease protection assays, RNA fingerprinting, polymerase chain reaction, ligase chain reaction, Qbeta replicase, isothermal amplification method, strand displacement amplification, transcription based amplification systems, nuclease protection (S1 nuclease or RNAse protection assays), SAGE as well as methods disclosed in International Publication Nos. WO 88/10315 and WO 89/06700, and International Applications Nos. PCT/US87/00880 and PCT/US89/01025; herein incorporated by reference in their entireties.

A standard Northern blot assay can be used to ascertain an RNA transcript size, identify alternatively spliced RNA transcripts, and the relative amounts of mRNA in a sample, in accordance with conventional Northern hybridization techniques known to those persons of ordinary skill in the art. In Northern blots, RNA samples are first separated by size by electrophoresis in an agarose gel under denaturing conditions. The RNA is then transferred to a membrane, cross-linked, and hybridized with a labeled probe. Nonisotopic or high specific activity radiolabeled probes can be used, including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radiolabelled cDNA, either containing the full-length, single stranded DNA or a fragment of that DNA sequence may be at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. Isotopes that can be used include, but are not limited to ³H, ¹⁴C, ³²P, ³⁵S, ³⁶Cl, ³⁵Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

Nuclease protection assays (including both ribonuclease protection assays and Si nuclease assays) can be used to detect and quantitate specific mRNAs. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

The ribonuclease protection assay, which is the most common type of nuclease protection assay, requires the use of RNA probes. Oligonucleotides and other single-stranded DNA probes can only be used in assays containing S1 nuclease. The single-stranded, antisense probe must typically be completely homologous to target RNA to prevent cleavage of the probe:target hybrid by nuclease.

Serial Analysis Gene Expression (SAGE), can also be used to determine RNA abundances in a cell sample. See, e.g., Velculescu et al., 1995, Science 270:484-7; Carulli, et al., 1998, Journal of Cellular Biochemistry Supplements 30/31:286-96; herein incorporated by reference in their entireties. SAGE analysis does not require a special device for detection, and is one of the preferable analytical methods for simultaneously detecting the expression of a large number of transcription products. First, poly A⁺ RNA is extracted from cells. Next, the RNA is converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptoavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a read-out of consecutive ditags of constant length. The presence of mRNA corresponding to each tag can then be identified from the nucleotide sequence of the clone and information on the sequence tags.

Quantitative reverse transcriptase PCR (qRT-PCR) can also be used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

A more recent variation of the RT-PCR technique is the real time quantitative PCR, which measures PCR product accumulation through a dual-labeled fluorigenic probe (i.e., TAQMAN probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Detecting Biomarker Proteins, Polypeptides, and Peptides

In one embodiment, the expression levels of biomarkers are determined by measuring protein, polypeptide, or peptide levels of the biomarkers. Assays based on the use of antibodies that specifically recognize the proteins, polypeptide fragments, or peptides of the biomarkers may be used for the measurement. Such assays include, but are not limited to, immunohistochemistry (IHC), western blotting, enzyme-linked immunosorbent assay (ELISA), radioimmunoassays (RIA), “sandwich” immunoassays, fluorescent immunoassays, immunoprecipitation assays, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 31 42, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prey. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278(2):123-131 (2000)), or a chromatography column, etc; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as ²H, ¹⁴C, ³²P, or ¹²⁵I, a fluorescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. and Cytochem., 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

It may be useful in the practice of the invention to fractionate biological samples, e.g., to enrich samples for lower abundance proteins to facilitate detection of biomarkers, or to partially purify biomarkers isolated from biological samples to generate specific antibodies to biomarkers. There are many ways to reduce the complexity of a sample based on the binding properties of the proteins in the sample, or the characteristics of the proteins in the sample.

In one embodiment, a sample can be fractionated according to the size of the proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of biomarkers.

In another embodiment, a sample can be fractionated by anion exchange chromatography. Anion exchange chromatography allows fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of biomarkers in a sample that are more negatively charged from other types of biomarkers. Proteins that are eluted with an eluant having a high pH are likely to be weakly negatively charged, and proteins that are eluted with an eluant having a low pH are likely to be strongly negatively charged. Thus, in addition to reducing complexity of a sample, anion exchange chromatography separates proteins according to their binding characteristics.

In yet another embodiment, a sample can be fractionated by heparin chromatography. Heparin chromatography allows fractionation of the biomarkers in a sample also on the basis of affinity interaction with heparin and charge characteristics. Heparin, a sulfated mucopolysaccharide, will bind biomarkers with positively charged moieties, and a sample can be sequentially eluted with eluants having different pH's or salt concentrations. Biomarkers eluted with an eluant having a low pH are more likely to be weakly positively charged. Biomarkers eluted with an eluant having a high pH are more likely to be strongly positively charged. Thus, heparin chromatography also reduces the complexity of a sample and separates biomarkers according to their binding characteristics.

In yet another embodiment, a sample can be fractionated by isolating proteins that have a specific characteristic, e.g. glycosylation. For example, a CSF sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

In yet another embodiment, a sample can be fractionated using a sequential extraction protocol. In sequential extraction, a sample is exposed to a series of adsorbents to extract different types of biomarkers from a sample. For example, a sample is applied to a first adsorbent to extract certain proteins, and an eluant containing non-adsorbent proteins (i.e., proteins that did not bind to the first adsorbent) is collected. Then, the fraction is exposed to a second adsorbent. This further extracts various proteins from the fraction. This second fraction is then exposed to a third adsorbent, and so on.

Any suitable materials and methods can be used to perform sequential extraction of a sample. For example, a series of spin columns comprising different adsorbents can be used. In another example, a multi-well comprising different adsorbents at its bottom can be used. In another example, sequential extraction can be performed on a probe adapted for use in a gas phase ion spectrometer, wherein the probe surface comprises adsorbents for binding biomarkers. In this embodiment, the sample is applied to a first adsorbent on the probe, which is subsequently washed with an eluant. Biomarkers that do not bind to the first adsorbent are removed with an eluant. The biomarkers that are in the fraction can be applied to a second adsorbent on the probe, and so forth. The advantage of performing sequential extraction on a gas phase ion spectrometer probe is that biomarkers that bind to various adsorbents at every stage of the sequential extraction protocol can be analyzed directly using a gas phase ion spectrometer.

In yet another embodiment, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

Prior to gas phase ion spectrometry analysis, it may be desirable to cleave biomarkers in the spot into smaller fragments using cleaving reagents, such as proteases (e.g., trypsin). The digestion of biomarkers into small fragments provides a mass fingerprint of the biomarkers in the spot, which can be used to determine the identity of the biomarkers if desired.

In yet another embodiment, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

Optionally, a biomarker can be modified before analysis to improve its resolution or to determine its identity. For example, the biomarkers may be subject to proteolytic digestion before analysis. Any protease can be used. Proteases, such as trypsin, that are likely to cleave the biomarkers into a discrete number of fragments are particularly useful. The fragments that result from digestion function as a fingerprint for the biomarkers, thereby enabling their detection indirectly. This is particularly useful where there are biomarkers with similar molecular masses that might be confused for the biomarker in question. Also, proteolytic fragmentation is useful for high molecular weight biomarkers because smaller biomarkers are more easily resolved by mass spectrometry. In another example, biomarkers can be modified to improve detection resolution. For instance, neuraminidase can be used to remove terminal sialic acid residues from glycoproteins to improve binding to an anionic adsorbent and to improve detection resolution. In another example, the biomarkers can be modified by the attachment of a tag of particular molecular weight that specifically binds to molecular biomarkers, further distinguishing them. Optionally, after detecting such modified biomarkers, the identity of the biomarkers can be further determined by matching the physical and chemical characteristics of the modified biomarkers in a protein database (e.g., SwissProt).

After preparation, biomarkers in a sample are typically captured on a substrate for detection. Traditional substrates include antibody-coated 96-well plates or nitrocellulose membranes that are subsequently probed for the presence of the proteins. Alternatively, protein-binding molecules attached to microspheres, microparticles, microbeads, beads, or other particles can be used for capture and detection of biomarkers. The protein-binding molecules may be antibodies, peptides, peptoids, aptamers, small molecule ligands or other protein-binding capture agents attached to the surface of particles. Each protein-binding molecule may comprise a “unique detectable label,” which is uniquely coded such that it may be distinguished from other detectable labels attached to other protein-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes. See, e.g., U.S. Pat. No. 5,981,180, U.S. Pat. No. 7,445,844, U.S. Pat. No. 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp.114-122; Nanobiotechnology Protocols Methods in Molecular Biology, 2005, Volume 303; herein incorporated by reference in their entireties).

In another example, biochips can be used for capture and detection of proteins. Many protein biochips are described in the art. These include, for example, protein biochips produced by Packard BioScience Company (Meriden Conn.), Zyomyx (Hayward, Calif.) and Phylos (Lexington, Mass.). In general, protein biochips comprise a substrate having a surface. A capture reagent or adsorbent is attached to the surface of the substrate. Frequently, the surface comprises a plurality of addressable locations, each of which location has the capture reagent bound there. The capture reagent can be a biological molecule, such as a polypeptide or a nucleic acid, which captures other biomarkers in a specific manner. Alternatively, the capture reagent can be a chromatographic material, such as an anion exchange material or a hydrophilic material. Examples of such protein biochips are described in the following patents or patent applications: U.S. Pat. No. 6,225,047 (Hutchens and Yip, “Use of retentate chromatography to generate difference maps,” May 1, 2001), International publication WO 99/51773 (Kuimelis and Wagner, “Addressable protein arrays,” Oct. 14, 1999), International publication WO 00/04389 (Wagner et al., “Arrays of protein-capture agents and methods of use thereof,” Jul. 27, 2000), International publication WO 00/56934 (Englert et al., “Continuous porous matrix arrays,” Sep. 28, 2000).

In general, a sample containing the biomarkers is placed on the active surface of a biochip for a sufficient time to allow binding. Then, unbound molecules are washed from the surface using a suitable eluant. In general, the more stringent the eluant, the more tightly the proteins must be bound to be retained after the wash. The retained protein biomarkers now can be detected by any appropriate means, for example, mass spectrometry, fluorescence, surface plasmon resonance, ellipsometry or atomic force microscopy.

Mass spectrometry, and particularly SELDI mass spectrometry, is a particularly useful method for detection of the biomarkers of this invention. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the biomarkers of this invention. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

Analysis of Biomarker Data

Biomarker data may be analyzed by a variety of methods to identify biomarkers and determine the statistical significance of differences in observed levels of biomarkers between test and reference expression profiles in order to evaluate whether a patient has ASD. In certain embodiments, patient data is analyzed by one or more methods including, but not limited to, multivariate linear discriminant analysis (LDA), receiver operating characteristic (ROC) analysis, principal component analysis (PCA), ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; McLachlan (2004) Discriminant Analysis and Statistical Pattern Recognition. Wiley Interscience; Zweig et al. (1993) Clin. Chem. 39:561-577; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif., USA; English et al. (2009) J. Biomed. Inform. 42(2):287-295; Zhang (2007) Bioinformatics 8: 230; Shen-Orr et al. (2010) Journal of Immunology 184:144-130; Qiu et al. (2011) Nat. Biotechnol. 29(10):886-891; Ru et al. (2006) J. Chromatogr. A. 1111(2):166-174, Jolliffe Principal Component Analysis (Springer Series in Statistics, 2^{nd} edition, Springer, N.Y., 2002), Koren et al. (2004) IEEE Trans Vis Comput Graph 10:459-470; herein incorporated by reference in their entireties.)

E. Kits for Measuring ASD Biomarkers

In yet another aspect, the invention provides kits for diagnosing ASD, wherein the kits can be used to detect the biomarkers of the present invention. For example, the kits can be used to detect any one or more of the biomarkers described herein, which are differentially expressed in samples of an ASD patient and normal subjects. The kit may include one or more agents for detection of biomarkers, a container for holding a biological sample isolated from a human subject suspected of having ASD; and printed instructions for reacting agents with the biological sample or a portion of the biological sample to detect the presence or amount of at least one ASD biomarker in the biological sample. The agents may be packaged in separate containers. The kit may further comprise one or more control reference samples and reagents for performing an immunoassay or microarray analysis. In addition, the kit may include agents for detecting one or more genetic markers associated with ASD, as described herein. Biomarkers can be used together in any combination with one or more genetic markers and/or in combination with clinical parameters for diagnosis of ASD.

In certain embodiments, the kit comprises at least one agent for measuring the level of at least one biomarker of interest, such as a gene or RNA transcripts of a gene, including, but not limited to, ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, and ZDHHC23; or a gene product thereof (e.g., protein or peptide).

In one embodiment, the kit comprises agents for detecting one or more biomarkers selected from the group consisting of an ACTN2 polynucleotide, a ATP2B2 polynucleotide, a BCAS1 polynucleotide, a CAMK2A polynucleotide, a CNTNAP4 polynucleotide, a DGKZ polynucleotide, a DLGAP2 polynucleotide, a DLGAP3 polynucleotide, a DYNLL1 polynucleotide, a GDA polynucleotide, a GRIA1 polynucleotide, a GRIK3 polynucleotide, a GRIN2A polynucleotide, a GRIN2B polynucleotide, a HTR2C polynucleotide, a KCNA4 polynucleotide, a KCNJ2 polynucleotide, a KCNJ4 polynucleotide, a LDB3 polynucleotide, a LPL polynucleotide, a NRXN2 polynucleotide, a PGM5 polynucleotide, a PTPRN polynucleotide, a S100A3 polynucleotide, a SCN1A polynucleotide, a SHANK2 polynucleotide, a SHANK3 polynucleotide, a TBR1 polynucleotide, a TJAP1 polynucleotide, and a ZDHHC23 polynucleotide.

In certain embodiments, the kit comprises a microarray for analysis of a plurality of biomarker polynucleotides. An exemplary microarray included in the kit comprises an oligonucleotide that hybridizes to a ACTN2 polynucleotide, an oligonucleotide that hybridizes to a ATP2B2 polynucleotide, an oligonucleotide that hybridizes to a BCAS1 polynucleotide, an oligonucleotide that hybridizes to a CAMK2A polynucleotide, an oligonucleotide that hybridizes to a CNTNAP4 polynucleotide, an oligonucleotide that hybridizes to a DGKZ polynucleotide, an oligonucleotide that hybridizes to a DLGAP2 polynucleotide, an oligonucleotide that hybridizes to a DLGAP3 polynucleotide, an oligonucleotide that hybridizes to a DYNLL1 polynucleotide, an oligonucleotide that hybridizes to a GDA polynucleotide, an oligonucleotide that hybridizes to a GRIA1 polynucleotide, an oligonucleotide that hybridizes to a GRIK3 polynucleotide, an oligonucleotide that hybridizes to a GRIN2A polynucleotide, an oligonucleotide that hybridizes to a GRIN2B polynucleotide, an oligonucleotide that hybridizes to a HTR2C polynucleotide, an oligonucleotide that hybridizes to a KCNA4 polynucleotide, an oligonucleotide that hybridizes to a KCNJ2 polynucleotide, an oligonucleotide that hybridizes to a KCNJ4 polynucleotide, an oligonucleotide that hybridizes to a LDB3 polynucleotide, an oligonucleotide that hybridizes to a LPL polynucleotide, an oligonucleotide that hybridizes to a NRXN2 polynucleotide, an oligonucleotide that hybridizes to a PGM5 polynucleotide, an oligonucleotide that hybridizes to a PTPRN polynucleotide, an oligonucleotide that hybridizes to a S100A3 polynucleotide, an oligonucleotide that hybridizes to a SCN1A polynucleotide, an oligonucleotide that hybridizes to a SHANK2 polynucleotide, an oligonucleotide that hybridizes to a SHANK3 polynucleotide, an oligonucleotide that hybridizes to a TBR1 polynucleotide, an oligonucleotide that hybridizes to a TJAP1 polynucleotide, and an oligonucleotide that hybridizes to a ZDHHC23 polynucleotide.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing ASD.

The kits of the invention have a number of applications. For example, the kits can be used to determine if a subject has ASD. In another example, the kits can be used to determine if a patient should be treated for ASD, for example, with behavior training, occupational therapy, or special education courses. In a further example, the kits can be used to identify compounds that modulate expression of one or more of the biomarkers in in vitro or in vivo animal models to determine the effects of treatment.

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Integrated Systems Analysis Reveals A Molecular Network Underlying Autism Spectrum Disorders

Introduction

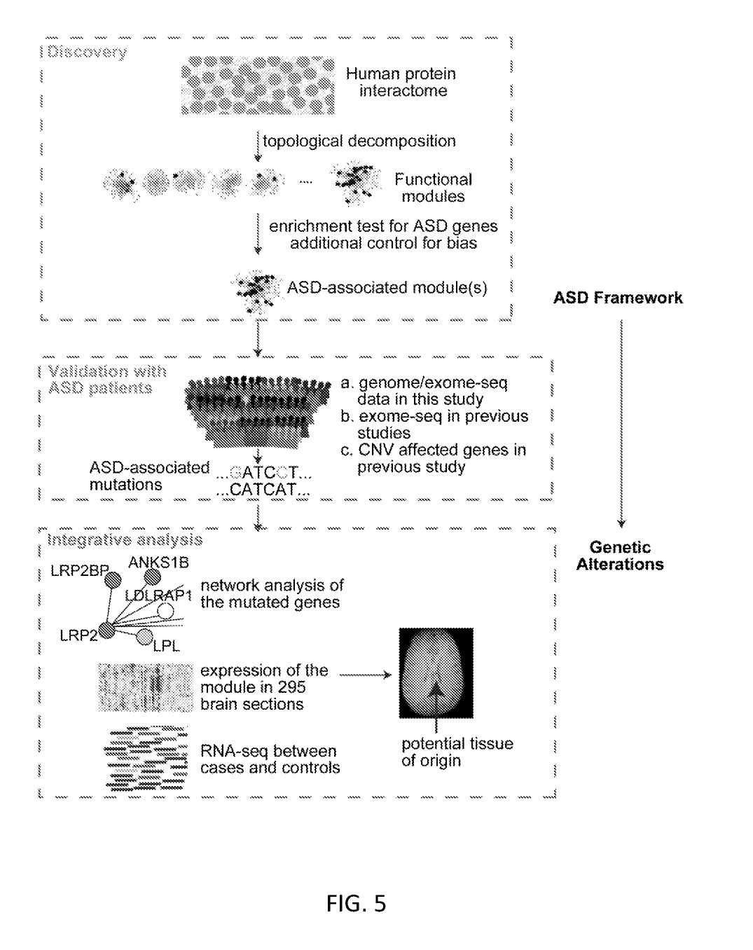

Herein we describe a systems biology approach (FIG. 5) to unravel the natural organization of physically interacting proteins implicated in ASD. We analyzed the human protein interactome to detect a protein module strongly enriched for biological processes relevant to ASD etiology. The module is frequently mutated in patients with autism, which was further validated in a large patient cohort and by our own independent sequencing studies. Network and transcriptome analyses of this ASD module collectively revealed that the corpus callosum is likely a potential tissue of origin underlying ASD, in line with morphological alterations that have been described for patients with an ASD (Boger-Megiddo et al. (2006) J. Autism Dev. Disord. 36(6):733-739; Frazier et al. (2012) J. Autism Dev. Disord. 42(11):2312-2322).

Results

Modularization of the Human Protein Interactome

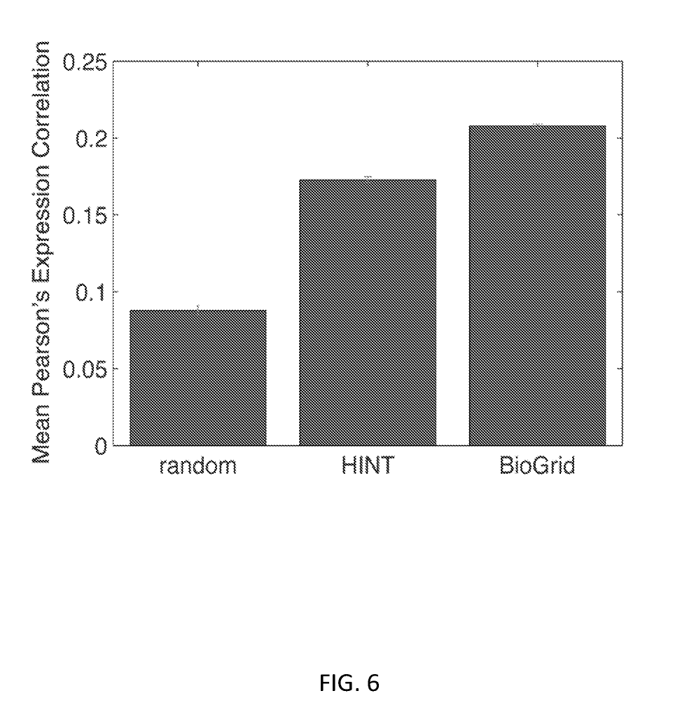

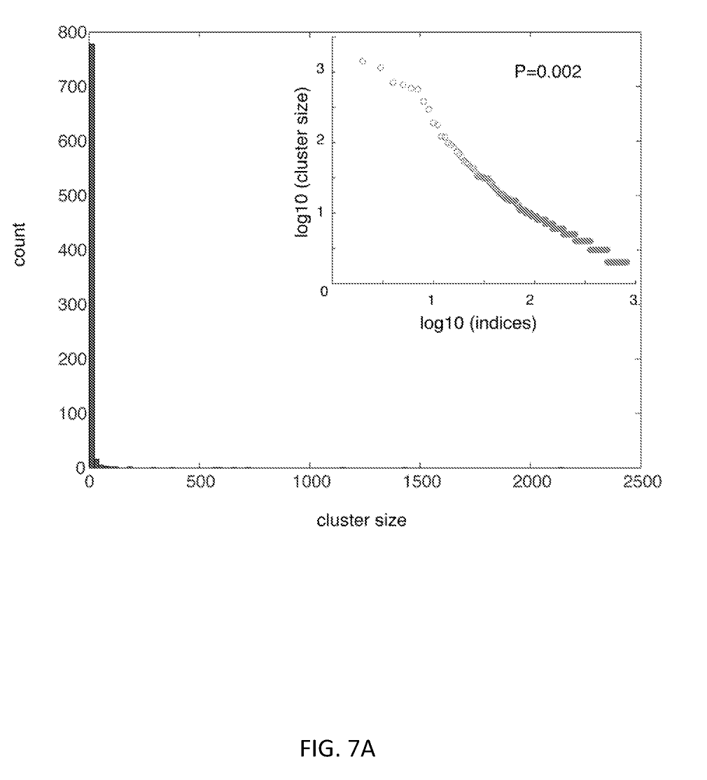

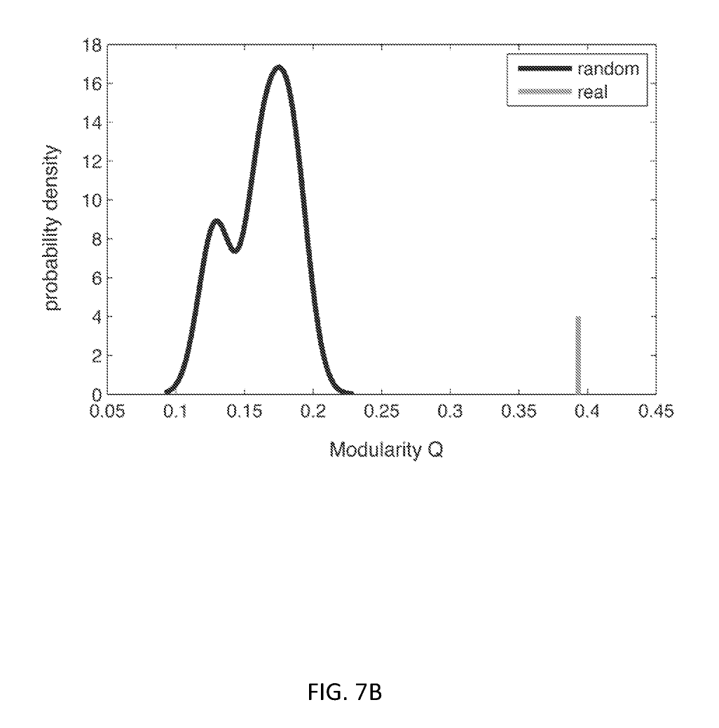

We first generated a new topological protein interaction network using the most comprehensive human protein interactome from BioGrid (Stark et al. (2011) Nucleic Acids Res. 39:D698-704) comprising 13039 genes and 69113 curated interactions. Since interacting proteins are presumably co-expressed, the quality of these protein interactions was often analyzed by co-expression analysis (Yu et al. (2008) Science 322:104-110, herein incorporated by reference). We found significantly increased gene co-expression from this dataset relative to a set of previously benchmarked interacting proteins (Das & Yu (2012) BMC Syst. Biol. 6:92) and also to randomly paired proteins (FIG. 6), demonstrating high quality of this human protein interactome dataset. We then topologically clustered the proteins that constituted the network into highly interacting modules using a parameter-free algorithm that was specifically designed for detecting community structures in a large-scale network (Blondel et al. (2008) Fast unfolding of communities in large networks, Journal of Statistical Mechanics-Theory and Experiment). By maximizing the score for network modularity, the human interactome was decomposed into 817 topological modules of non-uniform sizes (FIG. 7A). Within each module the proteins tightly interacted with each other, but sparsely with proteins in other modules. This observed modularity of the human interactome was then tested against a set of shuffled networks of the same size by randomly rewiring existing interactions while maintaining the same number of interacting partners. None of the randomized networks achieved the same modularity observed from the network in this study (FIG. 7B), confirming the significance of these topological clusters (P<0.01, estimated from the 100 random shufflings).

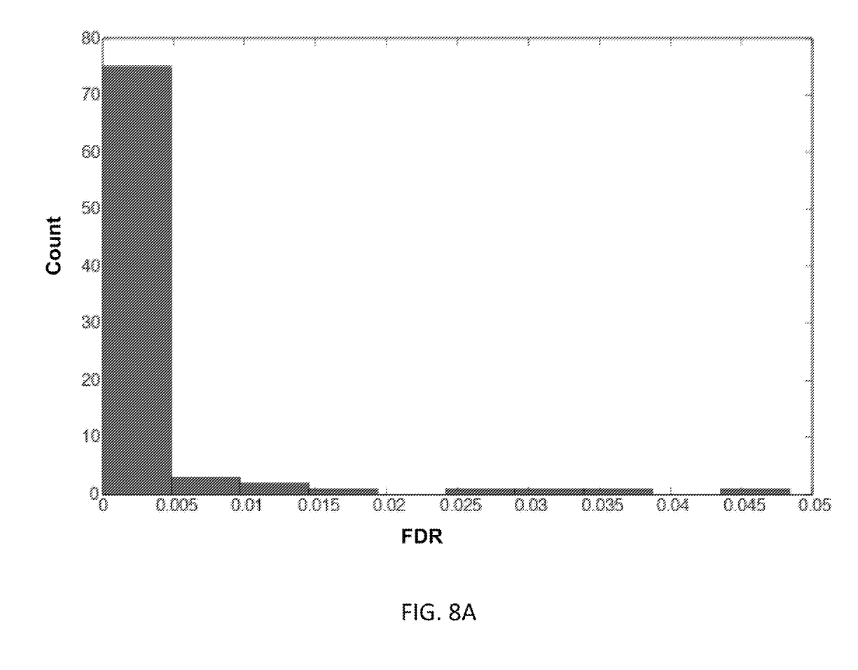

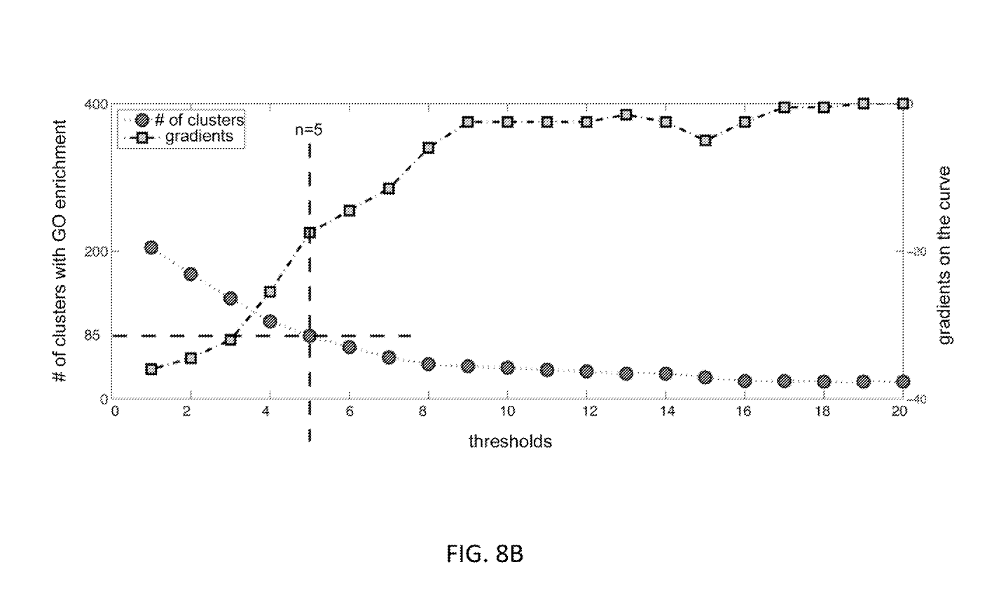

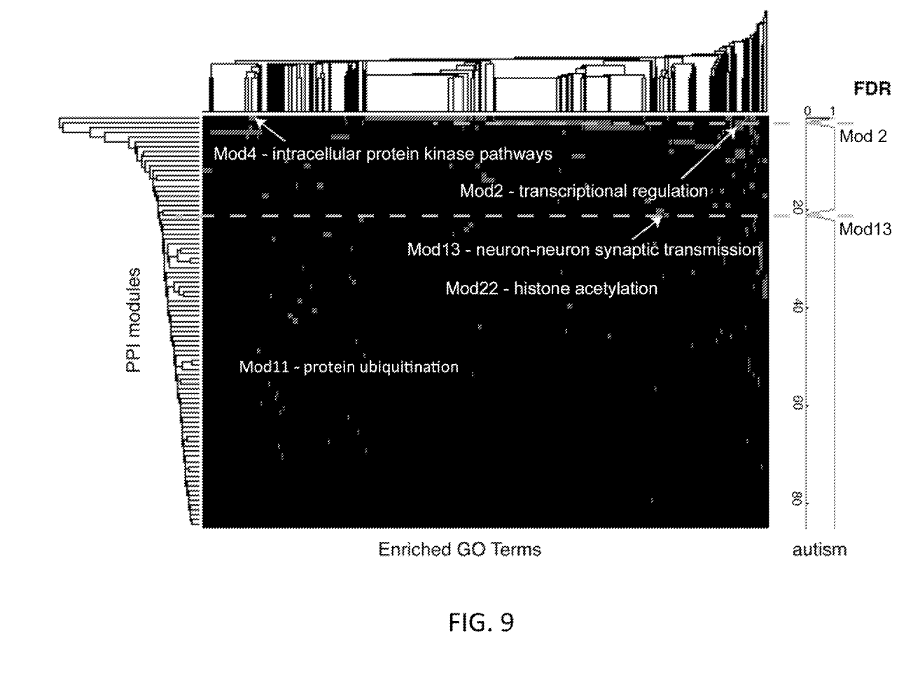

Gene Ontology (GO) enrichment analysis for the 192 topological modules containing more than 5 genes (FIG. 8) revealed 85 modules that showed significant enrichment for at least one GO term (FDR<0.05, hypergeometric test). The enrichment was highly significant for most of the modules (FDR≦5e-3, FIG. 8), including module #22 for histone acetylation (FDR=5.3e-3), module #4 for kinase cascades (FDR=9.41231e-18), module #2 for DNA-dependent regulation (FDR=2.43e-237) and module #13 for synaptic transmission (FDR=2.77e-28). Overall, these observations revealed the modular architecture of the human protein interactome, with different modules organized for specific functions (FIG. 9).

A Protein Interaction Module is Associated with Autism

To determine if any of the modules are related to autism, we examined the 383 genes involved in ASD susceptibility from the SFARI Gene list (gene.sfari.org/autdb/) that were present in the network. Enrichment tests for each module in the network revealed that module #2 (1430 member genes, FDR=2.3e-3, hypergeometric test) and #13 (119 member genes, FDR=4.6e-11, hypergeometric test) showed significant enrichment. Module #2 was enriched for transcriptional regulation, including ASD-associated transcription factors and chromatin remodelers (FOXP2, MECP2, and CHD8, etc.), and module #13 encompassed many genes for synaptic transmission (SHANK2, SHANK3, NLGN1, NLGN3, etc., see GO enrichment test above). Given the substantially stronger enrichment for SFARI ASD genes in module #13 relative to module #2, in the remaining part of the study we focused on module #13 for its ASD implication and molecular function.

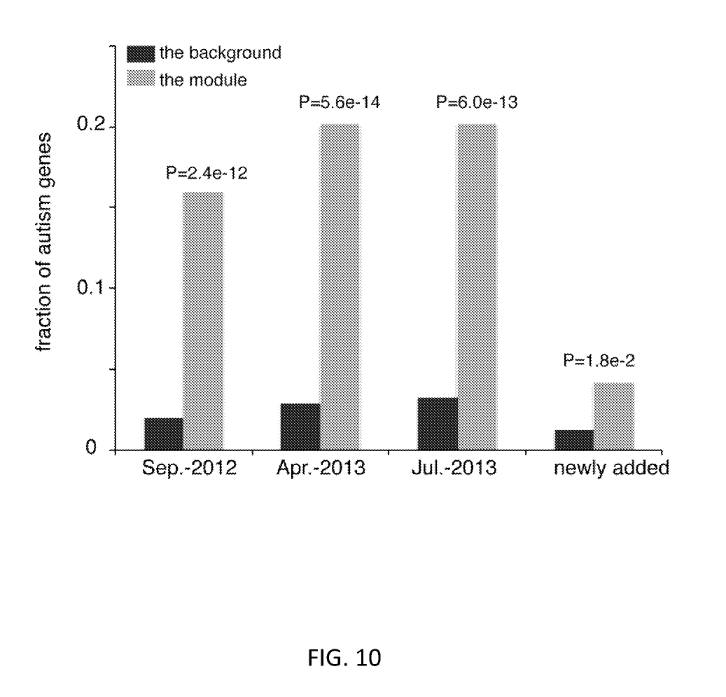

To determine that the observed enrichment for SFARI genes was not biased by unequal CDS (coding DNA sequences) length and GC content in the above comparison, we further performed 10000 sets of permutation tests. In each permutation we randomly sampled genes with indistinguishable CDS length and GC content from the SFARI genes, and we observed the same enrichment for module #13 (P<1e-5). The SFARI reference ASD gene list, although comprehensive, is likely to have potential curation bias. We therefore tested this module's enrichment for ASD genes using a variety of validation tests. We first tested whether the observed enrichment for ASD genes in module #13 was simply accounted for by its overall enrichment for synaptic genes. Of the total 1886 known synaptic genes from SynaptomeDB (Pirooznia et al. (2012) Bioinformatics 28:897-899), 1745 were present on the network. After removal of the synaptic genes from module #13, ASD non-synaptic genes were highly enriched in the module relative to those in the entire network or across the genome (14.8% vs 2.6% and 2.9%, respectively; P=1.64e-4, hypergeometric test). Furthermore, 5.44% (95/1745) of ASD genes were in the synaptic set for the entire network, but 21% (25/119) were in in module #13, a highly significant enrichment (P=1.27e-7, Fisher's exact test, for the ratio difference from the synaptic gene set). These comparisons collectively demonstrate that the ASD enrichment in module #13 cannot be attributed to only the synaptic genes in this module, but instead is due to a clustering of ASD genes in the module. Furthermore, the enrichment was also observed when testing ASD genes from different releases of the SFARI curated database (P≦1e-10, FIG. 10).

We next analyzed the association of module #13 with ASD using data from several unbiased genomic studies. To account for any potential bias in CDS length or GC content, all comparisons were based on a set of 9,782 genes with comparable CDS length and GC content with genes in module #13 (P=0.25 and 0.14, respectively, Wilcoxson ranksum test). We performed five independent tests using (1) all the genes whose exons were affected by de novo CNV events from three independent studies (Levy et al. (2011) Neuron 70: 886-897; Pinto et al. (2014) Nature 466:368-372; Sanders et al. (2011) Neuron 70: 863-885); (2) a list of 203 high-confidence genes affected by ASD-associated de novo CNVs detected in 181 individuals with autism (Noh et al. (2013) PLoS Genetics 9:e1003523); (3) 407 genes affected by rare CNV events associated with ASD (Pinto et al. (2010) Nature 466:368-372); (4) 70 genes affected by de novo loss-of-function mutations in ASD probands; (5) 379 genes affected by de novo missense mutations in ASD probands. As control gene sets for these analyses we also included: (6) 557 genes whose exons were affected by de novo CNVs identified from non-ASD individuals (Kirov et al. (2012) Mol. Psychiatry (2):142-153) or unaffected siblings (Levy et al. (2011) Neuron 70:886-897; Sanders et al. (2011) Neuron 70:863-885); (7) 109 genes with de novo missense mutations identified in unaffected siblings; (8) 148 and 52 genes with de novo silent mutations in ASD probands and unaffected siblings, respectively. All of the above de novo point mutations were from recent large-scale exome sequencing studies (Neale et al. (2012) Nature 485:242-245; O'Roak et al. (2012) Nature 485: 246-250; Sanders et al. (2012) Nature 485:237-241). The exact comparisons are shown in Tables 1A and 1B.

We observed that genes affected in ASD patients by the de novo CNVs (19.33% in the module versus 11.27% in the background, P=0.01, Fisher's exact test), the rare CNVs (5% in the module v.s. 2.1% in the background, P=0.048, Fisher's exact test) and the disruptive mutations (2.52% in the module v.s. 0.54% in the background, P=0.03, Fisher's exact test) each displayed a significant enrichment for this module, whereas the enrichment signal was absent from all types of mutations identified from non-ASD individuals and unaffected siblings, nor the silent mutations from ASD probands (See Tables 1A and 1B for the exact comparisons). Notably, although all ASD cohorts were enriched, the strongest enrichment signal was from the high-confidence CNV genes in ASD patients (Noh et al. (2013) PLoS Genetics 9:e1003523), where 14.29% of these genes were implicated in this module compared with 1.2% in the matched background (P=3.1e-13, Fisher's exact test). Lastly, the similar enrichment was also observed from a set of ASD-associated genes with syndromic mutations, or highly replicable genes in different GWAS patient cohorts (P=3.85e-6, Fisher's exact test, scored by SFARI Gene Module, category “S”). Overall, both curated data and data from genome-wide screening consistently support a significant association of module #13 with ASD. Our own sequencing as described in the section below provides further evidence for this module's involvement in ASD.

Module #13 was also more enriched for ASD genes (21% in the module) than genes involved in schizophrenia (10% in the module, Jia et al. (2010) Mol. Psychiatry 15(5):453-462) and intellectual disability (9.2% in the module, Parikshak et al. (2013) Cell 155:1008-1021), whereas no enrichment was observed for the Alzheimer's disease (Bertram et al, 2007) (P=0.28, Fisher's exact test). The increased overlap with schizophrenia and intellectual disability relative to Alzheimer's disease was expected given the shared molecular etiology among psychiatric disorders (Lee et al. (2013) Nature Genetics 45:984-994). Overall, this comparison suggests that the module is likely most specific towards ASD-related genes.

DNA Sequencing of ASD Patients Reveals an Enrichment of Rare Nonsynonymous Mutations in this Module

We sequenced postmortem brain DNAs collected from 25 ASD patients (all Europeans); in 19 subjects we sequenced the whole exomes (WES, >97X coverage) and in six the whole-genome (WGS, ˜35-40X coverage). In addition, we sequenced four genomes and one exome from non-autistic European individuals to control for the overall sequencing quality (see Tables 2-4). We first analyzed variants identified from the WES platform, and identified 153 non-synonymous variants that were mapped onto the module, among which 19.6% (30/153) were extremely rare and were not previously observed in the 1000 Genome dataset. Randomly sampling the same number of genes 10,000 times, with indistinguishable CDS length and GC content from those in this module, demonstrated a significant enrichment for the rare non-synonymous variants in this module (P=1.2e-3, with the expected fraction 12%). The same enrichment signal was also observed from the variants identified by WGS (P=2.5e-3, permutation test).

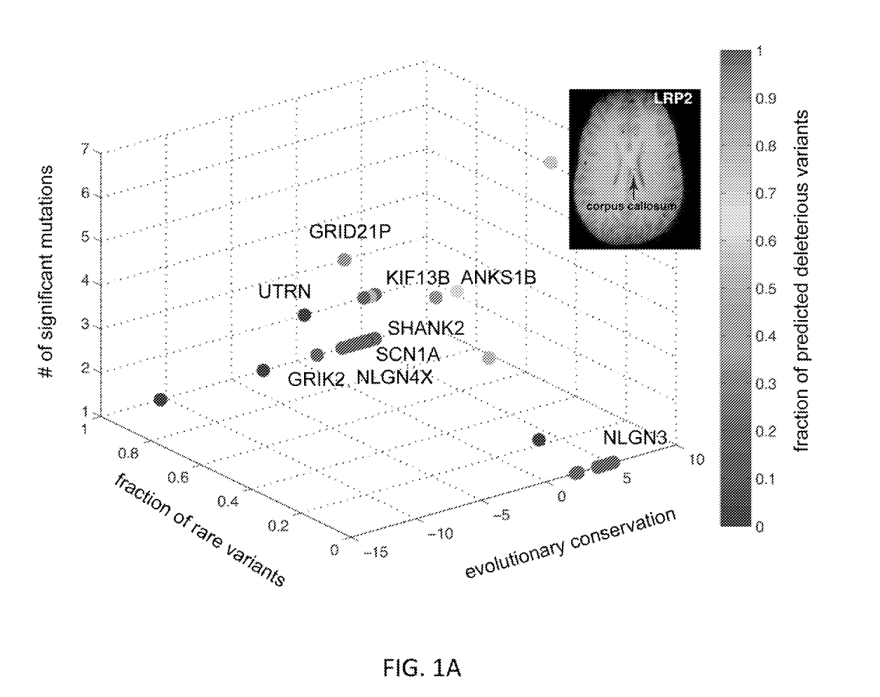

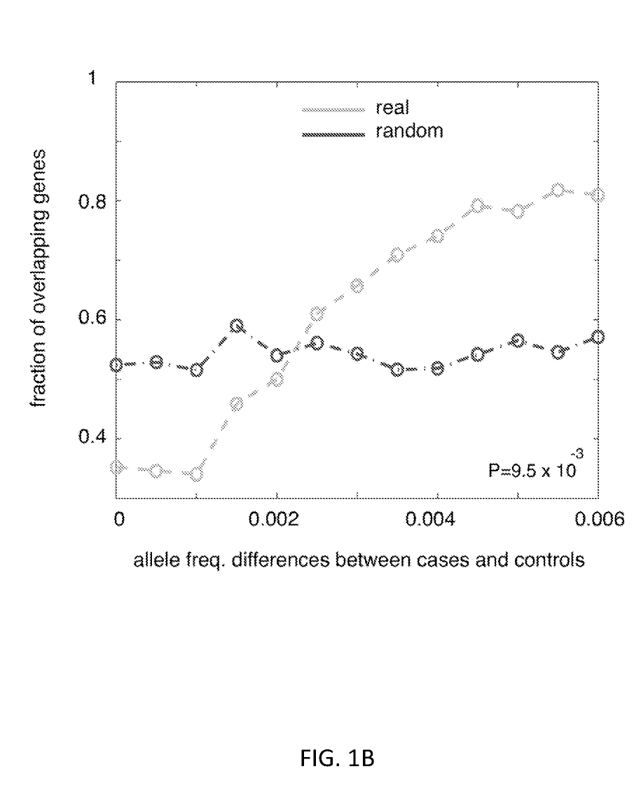

Excluding the variants also identified in the control subjects that were sequenced on the same platform, we considered 113 non-synonymous sites in this module collectively identified from WGS or WES. We compared their allele frequencies to those in the 1000 Genomes data set, both the entire global populations and the European populations, and from the 25 patients we identified a total of 38 genes affected by significant non-synonymous variants in this module with an expected false positive rate at 0.1 (determined by Fisher's exact test followed by Benjamini-Hochberg correction). The high gene overlap between WGS and WES was not expected by chance (P=0.03 by random permutation test). Furthermore, the identification of genes in our module was not affected by the CDS length of the identified genes relative to average CDS length in the module (P=0.16, Wilcoxon ranksum test). The identified genes and a summary of the variant information are shown in FIG. 1A. For example, LRP2 harbored seven distinct nonsynonymous mutations (z-axis, FIG. 1A), four of which are predicted to be deleterious by MutationTaster (Schwarz et al. (2010) Nature Methods 7:575-576). LRP2 has recently been identified as an ASD candidate gene (Ionita-Laza et al. (2012) Am. J. Hum. Genet. 90(6):1002-1013), whose clinical mutations cause the DonnaiBarrow syndrome (Kantarci et al. (2007) Nature Genetics 39:957-959) with underdeveloped or absent corpus callosum. This syndrome exhibits many autistic-like symptoms. FIG. 1A further underlines its tissue specificity in the corpus callosum using Brain Explorer (brain-map.org). Other well-characterized ASD-associated genes included SHANK2, SCN1A, NLGN4X and NLGN3 as well as several LRP2 interacting proteins (LRP2BP, ANKS1B). Overall, the affected loci in these genes were more likely to be both rare in the population (y-axis) and evolutionarily conserved (x-axis), suggesting their functional importance (FIG. 1A). We also noted that 28 genes of the 38 ASD candidates have not been described previously. To better support their association with this disease, we further examined their mouse mutant phenotypes in Mouse Genome Informatics (informatics.jax.org), and observed that 10 of the 28 new candidate genes displayed abnormal behavioral traits or a defective nervous system in their respective mouse mutants. For example, mouse mutants of 1) ANKS1B and KCNJ12 exhibited hyperactivity, 2) ERBB2IP hyporesponsive behavior to stimuli, 3) GRID2IP abnormal reflex and 4) SCN5A seizure.

Validation Using an Independent Patient Cohort

We next sought to further validate our observations in a larger patient cohort. An exome-sequencing dataset of 505 ASD cases and 491 controls, each of European ancestry and unrelated within the cohort, was analyzed (Liu et al. (2013) PLoS Genetics 9:e1003443). These samples had been sequenced using a separate sequencing platform (SOLiD) and the patients did not overlap with our sequenced cohort. A previous study examined this dataset but did not find any genes (or variants) significantly associated with ASD (Liu et al., supra). We compared the allele frequencies for each of nonsynonymous variant detected in this study, and found ˜95% of these variants had case-control frequency differences below 0.8%. We observed that genes with nonsynonymous variants with the highest allele frequency differences between cases and controls were more likely to be in the 38 module-specific candidate genes that we identified in our sequencing cohort (FIG. 1B), and this trend was not observed when we randomly sampled the same number of genes from the module for 10,000 times (P=9.5e-3, FIG. 1B). Furthermore, regression analysis on this dataset identified 16 genes in this module with the extreme imbalanced allele frequencies among the patient population (P<0.05), 14 were in the 38 candidate genes we identified (P=1.2e-6, hypergeometric test). Thus, this large-scale exome-sequencing data validated and extended our results.

Expression Specificity of the Module in the Corpus Callosum

We next examined expression of the genes in module#13 using the Allen Human Brain Atlas (Hawrylycz et al. (2012) Nature 489: 391-399), which describes the spatial gene expression across hundreds of neuroanatomically precise subdivisions as measured by microarray analyses of two individuals. Since the individuals exhibited high concordance in expression profiles across brain sections (Hawrylycz et al., supra), we averaged the gene expression data for each of the 295 anatomical brain sections.

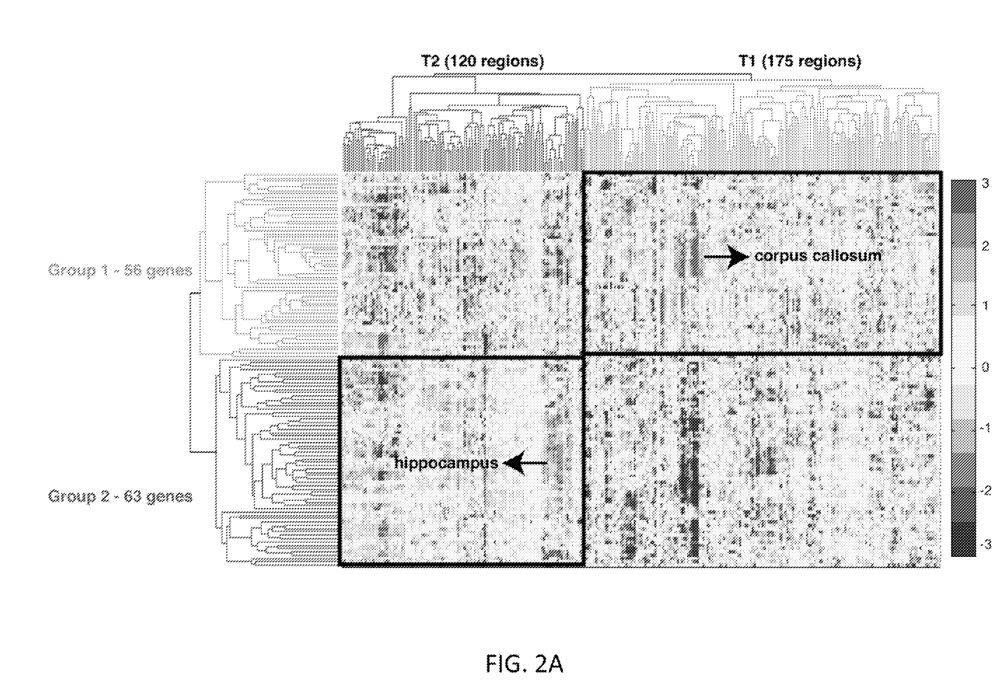

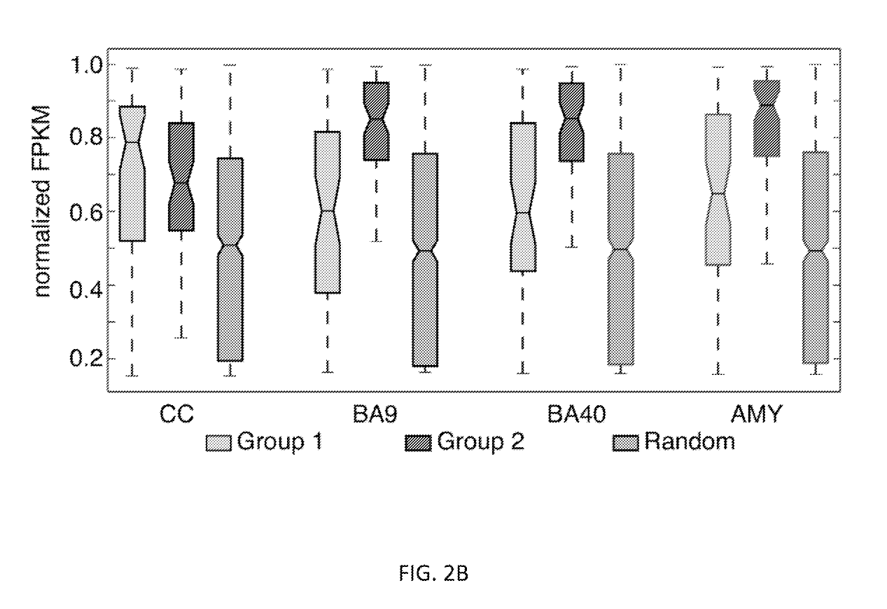

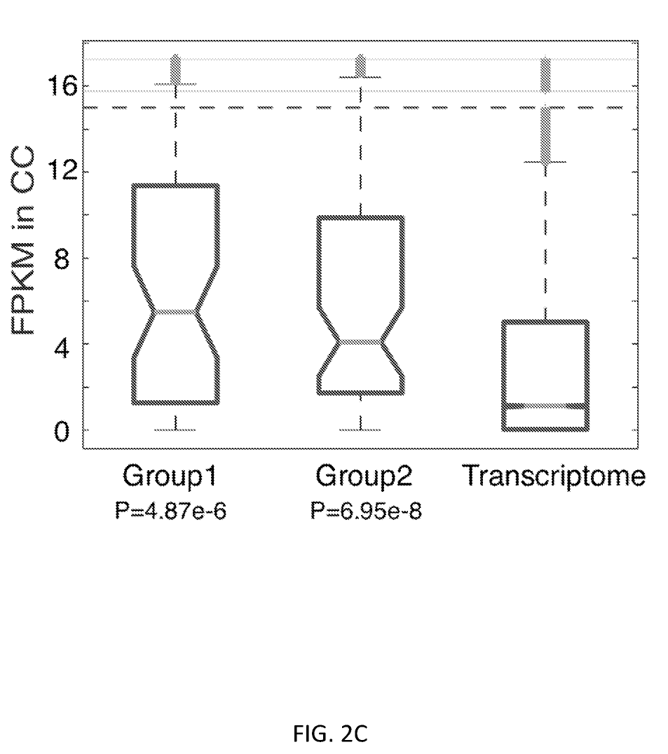

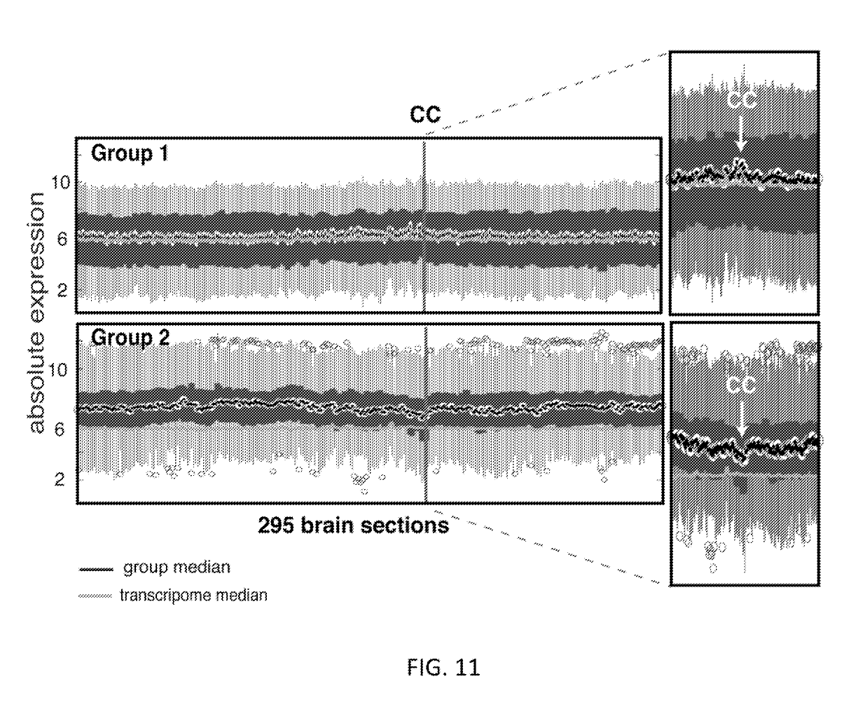

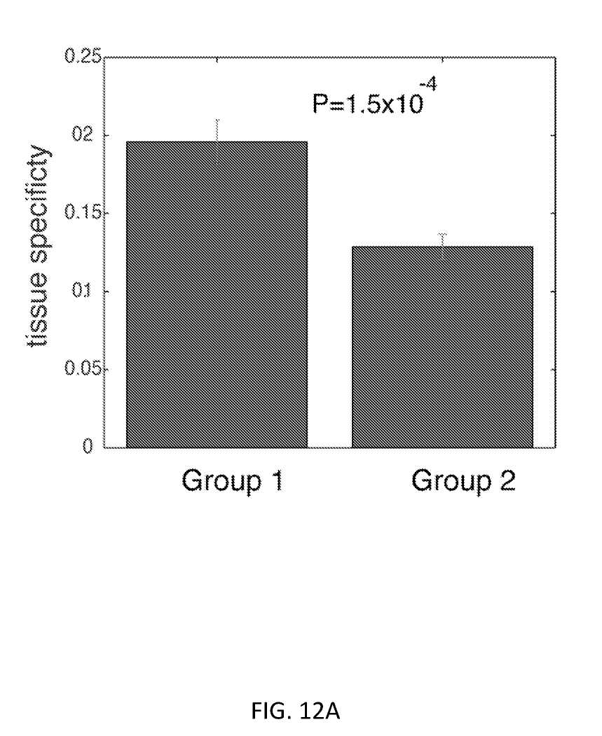

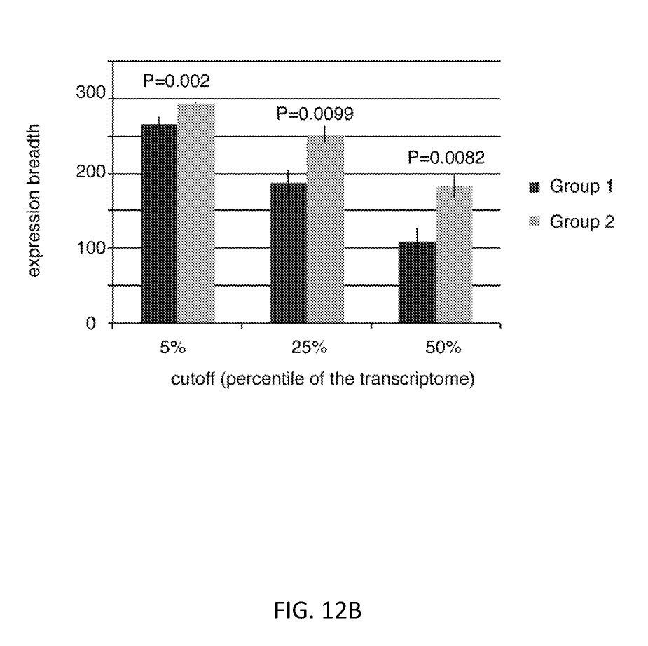

Most genes in module #13 were expressed across all brain sections (FIG. 11). However, hierarchical clustering of normalized gene expression across brain sections revealed two distinct spatial patterns with some heterogeneity apparent in each (FIG. 2A). Group 1 had 56 of 119 total genes preferentially expressed in 175 regions (T1 tissues in FIG. 2A), whereas the 63 genes of Group 2 had elevated expression in the other 120 brain regions (T2 tissues in FIG. 2A). Group 1 genes were strongly expressed in tissues associated with the corpus callosum (FIG. 2A, including LRP2 shown in FIG. 1A), which transfers motor, sensory and cognitive signals between the brain hemispheres. Group 2 genes (e.g. SHANK2 and SHANK3) were up-regulated in T2 regions, which encompassed neuron-rich regions, exemplified by the hippocampal formation, including CA 1/2/3/4 fields, subiculum and dentate gyms. Tissue enrichment was derived from relative expression of individual genes across brain sections; closer examination of their absolute expression in each brain section relative to the transcriptome background revealed that Group 1 expression levels were at background levels across most tissue types, but peaked in the corpus callosum (FIG. 11). Group 2 genes were highly expressed across all tissues, albeit their expression levels were slightly depressed in the corpus callosum (FIG. 11). Thus Group 2 genes were more ubiquitously expressed, and Group 1 genes were tissue-specific in the corpus callosum with an increased tissue specificity index (P=1.5e-4, Wilcoxon ranksum test) and decreased expression breadth (P<0.01, Wilcoxon ranksum test, FIG. 12).

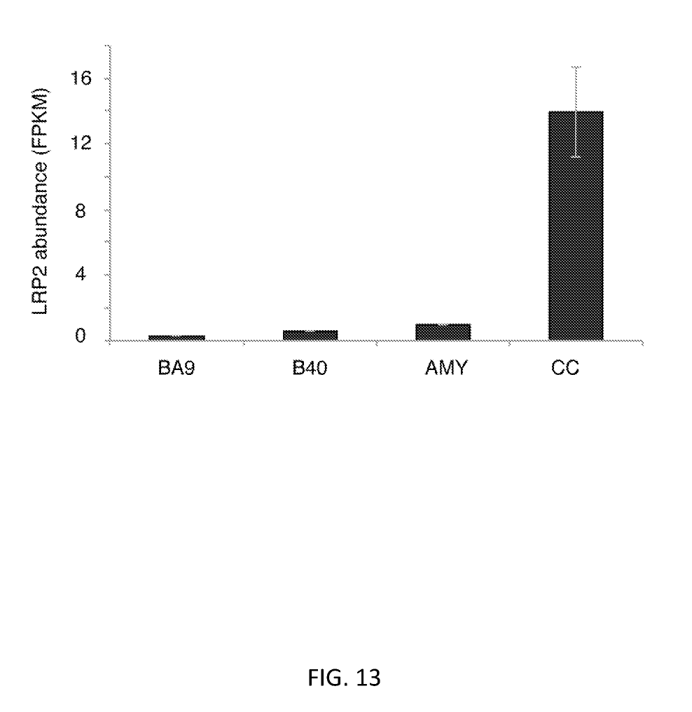

We further tested the tissue-specificity of expression patterns by RNA-sequencing (RNA-Seq) of postmortem human brain samples in two sets of experiments. First, we examined expression levels in four brain regions of one individual with no known disease. These regions were the dorsolateral prefrontal cortex (Brodmann Area 9, BA9), the parietal lobe (Brodmann Area 40, BA40), the amygdala (AMY), and the corpus callosum. BA9, BA40 and AMY are neuron-rich regions, while the corpus callosum is glial-rich. Consistent with the microarray results, Group 2 genes were highly expressed in all tissues (P<8e-7, Wilcoxon ranksum test, FIG. 2B) confirming their ubiquitous expression, and Group 1 genes showed the greatest up-regulation over the average transcriptome background in the corpus callosum (P<1.6e-6, Wilcoxon ranksum test, FIG. 2B) confirming their increased tissue specificity. These RNA-Seq experiments also confirmed the tissue specificity of LRP2 in the corpus callosum (FIG. 13), as expected from FIG. 1A. Secondly, to rule out individual variability, we also examined gene expression by RNA-Seq of the corpus callosum from 6 normal individuals (all young Caucasian males; the control subjects in our later RNA-Seq experiments). We found that both Group 1 and 2 genes were highly expressed in the corpus callosum relative to the transcriptome background (P<4.87e-6, Wilcoxon ranksum test, FIG. 2C). These results confirm that module #13 as a whole is highly expressed in the corpus callosum, the largest white matter structure in human brain.

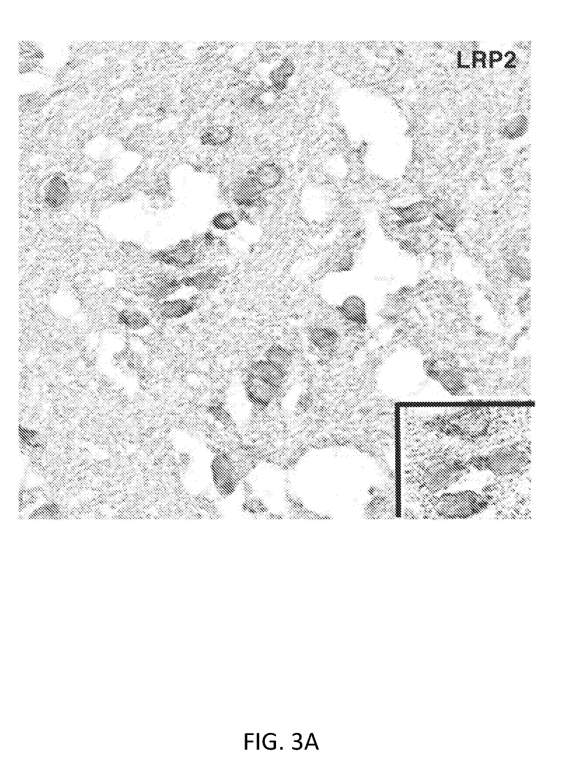

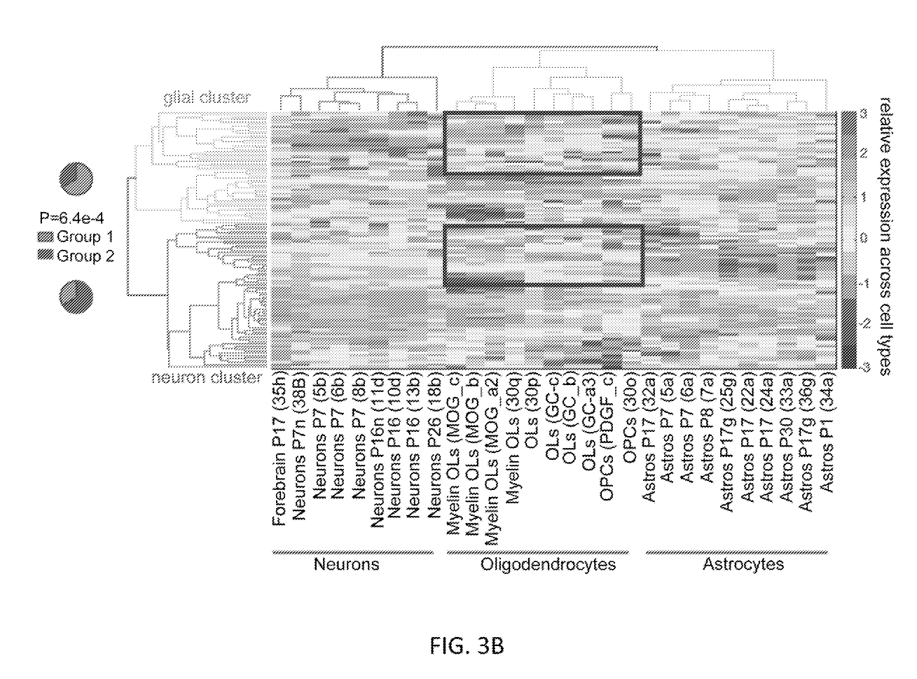

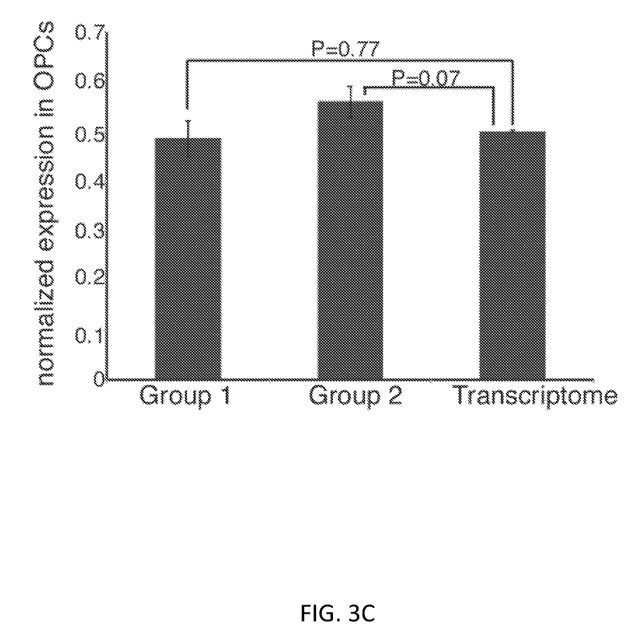

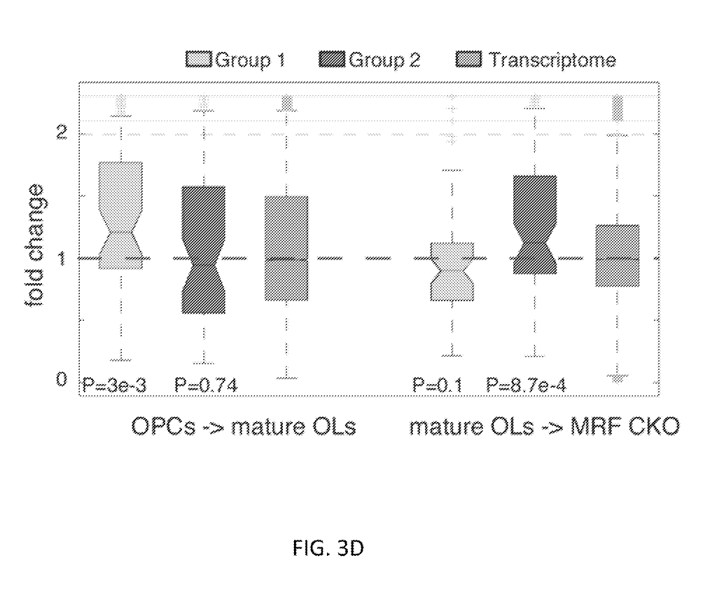

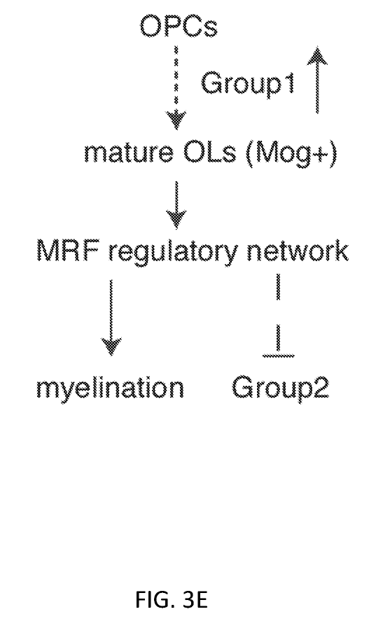

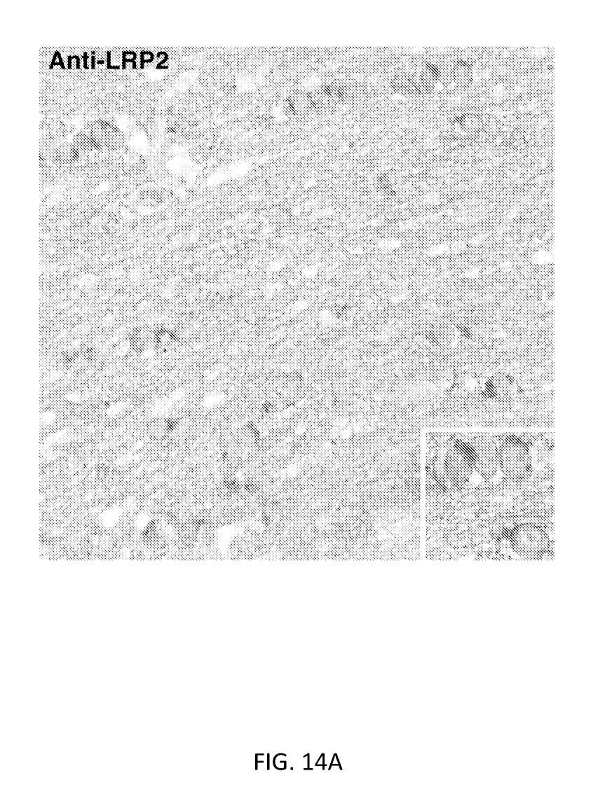

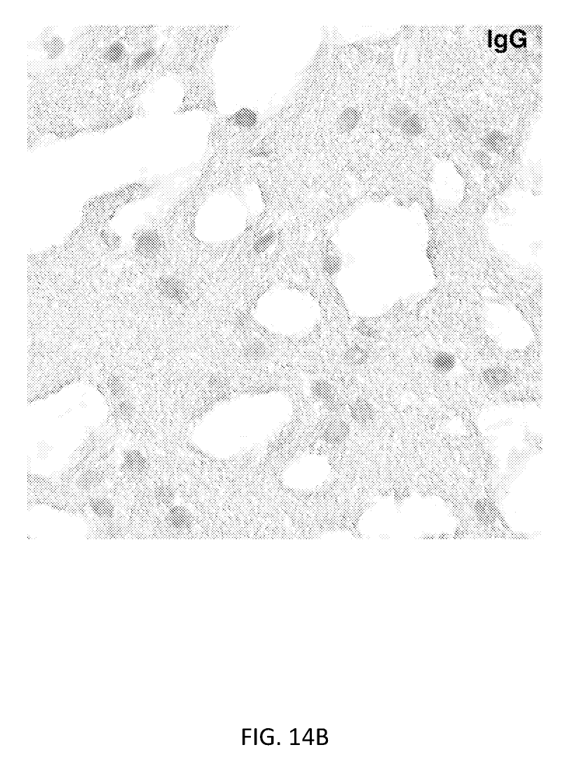

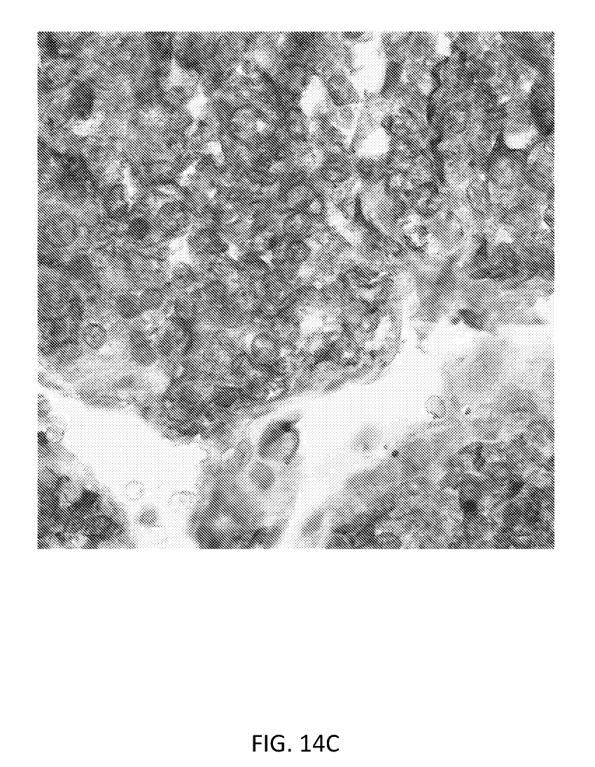

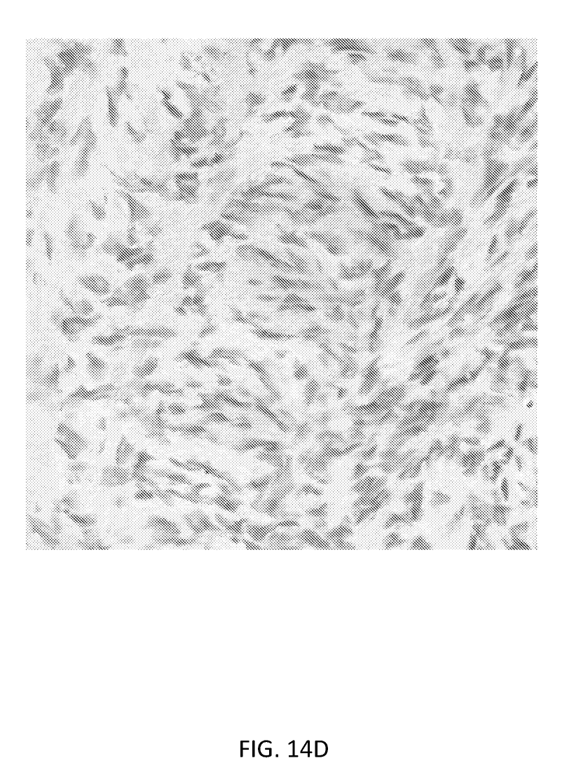

To further validate our results we performed immunohistochemical analyses for a Group 1 corpus-callosum specific gene (FIG. 13), LRP2, that also showed excessive mutation in our sequencing analyses (FIG. 1A). The experiment was performed in the postmortem corpus callosum tissue from one autism patient (FIG. 3A) and one control subject (FIG. 14). LRP2 protein was significantly expressed in the corpus callosum in both individuals, with no obvious difference between the normal and ASD subjects. As shown in FIG. 3A, the staining results further revealed that the human corpus callosum was predominantly populated by oligodendrocyte cells.

Given this fact, we next explored the function of this module in the oligodendrocytes by comparing gene expression of module #13 with other major cell types (neurons and astrocytes) in brain. Due to a lack of the cell-type expression data in human brain, we mapped module #13 onto their unambiguous mouse orthologs (the one-to-one orthology), and analyzed their cell-type expression (Cahoy et al. (2008) J. Neurosci. 28(1):264-278). Hierarchical clustering revealed that the mouse orthologs in our module formed two major clusters with expression enrichments in either neurons or in glial cells (i.e. oligodendrocytes and astrocytes, FIG. 3B). The expression profiles of glial cells were significantly enriched for Group 1 genes, and of neuronal cells for Group 2 genes (P=6.4e-4, chi-square test, FIG. 3B), suggesting that expression propensities of Group 1 and 2 in sections T1 and T2 (FIG. 2A), respectively, were largely due to their different compositions of glial cells and neurons. However, a portion of the genes in both the neuron and glial clusters showed common enrichment in oligodendrocytes, separating the cluster of the myelinating oligodendrocytes (myelin OLs, the sub-cluster on the x-axis, FIG. 3B) from the non-myelinating oligodendrocytes (the newly differentiated oligodendrocytes, OLs, and the oligodendrocyte precursor cells, OPCs, the sub-cluster on the x-axis, FIG. 3B). We thus hypothesized that the two subcomponents (Group 1 and 2) in the module are likely to be involved in the development of oligodendrocyte cells.

Using the data generated by Emery et al. (Emery et al. (2009) Cell 138:172-185), we next compared gene expression of the mouse orthologs of Group 1 and 2 genes in differentiating mouse culture systems. In cultured oligodendrocyte precursor cells (OPCs) the two gene groups did not show substantial expression changes relative to the transcriptome average (FIG. 3C). However, in the matured myelinating oligodendrocytes (MOG⁺), Group 1 genes exhibited marked up-regulation (P=3.0e-3, Wilcoxon ranksum test, FIG. 3D), whereas the Group 2 genes showed slight down-regulation with no statistical significance (P=0.74, Wilcoxon ranksum test). This indicates that up-regulation of Group 1 genes is associated with oligodendrocyte maturation.

In the same mature oligodendrocytes, we tested the expression of module #13 components using mouse knockouts (Emery et al. (2009), supra). The transcription factor, myelin gene regulatory factor (MRF), plays a central role in developing myelination capacity for oligodendrocyte cells and mice lacking MRF in the oligodendrocyte lineage show defects of myelination, accompanied by severe neurological abnormalities and postnatal lethality due to seizures (Emery et al., supra). In mouse oligodendrocytes with a conditional knockout of MRF (MRF^{fl}/^{fl}; Olig2^{wt/cre}), Group 2 genes exhibited a significant up-regulation relative to the transcriptome background (P=8.7e-4, Wilcoxon ranksum test, FIG. 3D), whereas Group 1 genes underwent down-regulation with marginal statistical significance (P=0.1, Wilcoxon ranksum test, FIG. 3D). This suggests that Group 2 genes are directly or indirectly suppressed by the master myelination factor MRF in the myelinating oligodendrocytes. Overall, given these observations, we propose that up-regulation of the Group 1 genes in our module is associated with, or likely contributes to, oligodendrocyte maturation from their precursor cells (OPSc). However, in the mature oligodendrocytes, myelination capacity is acquired by the MRF-mediated regulatory network, which also serves to suppress expression of the Group 2 genes (FIG. 3E).

Altered Gene Expression in the Corpus Callosum of ASD Patients Revealed by RNA-Sequencing

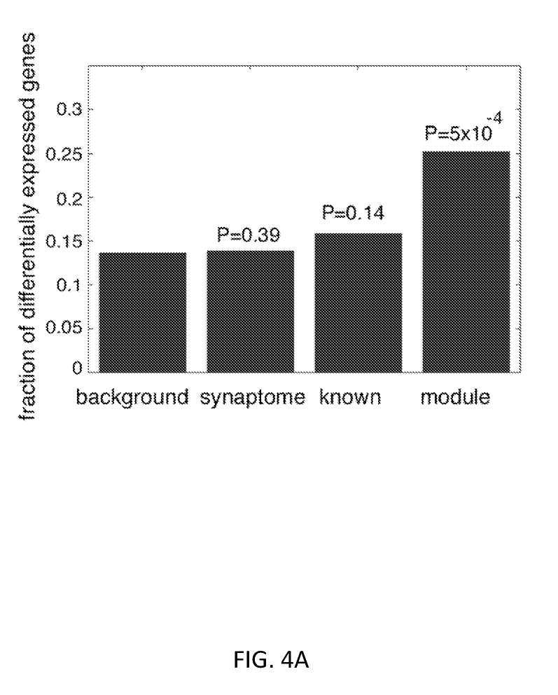

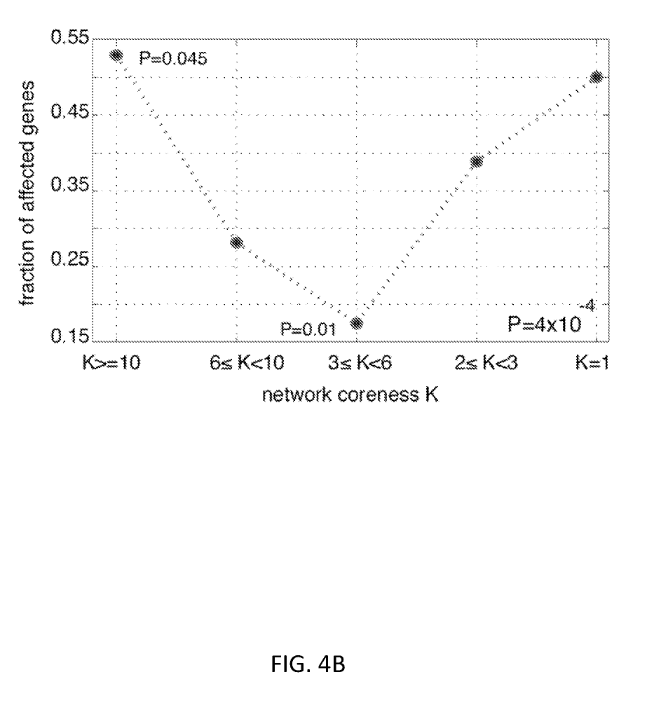

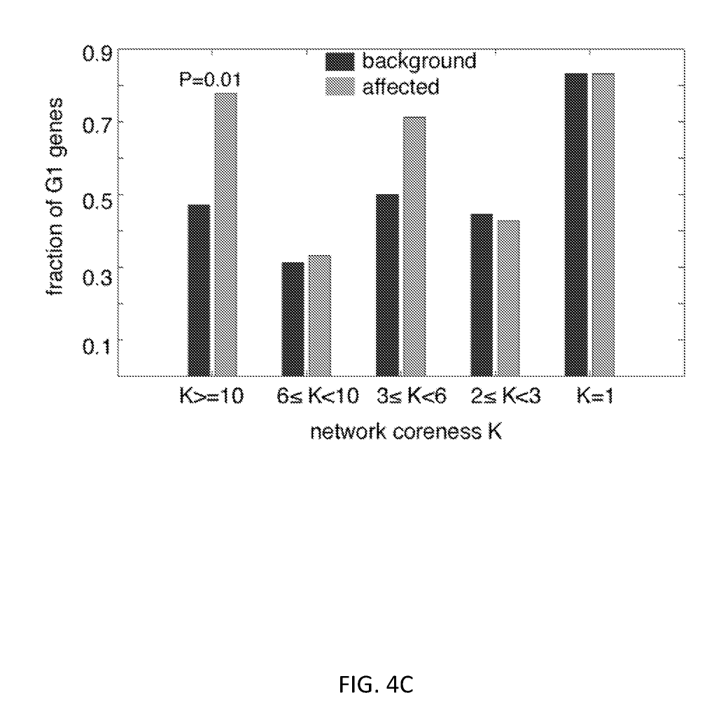

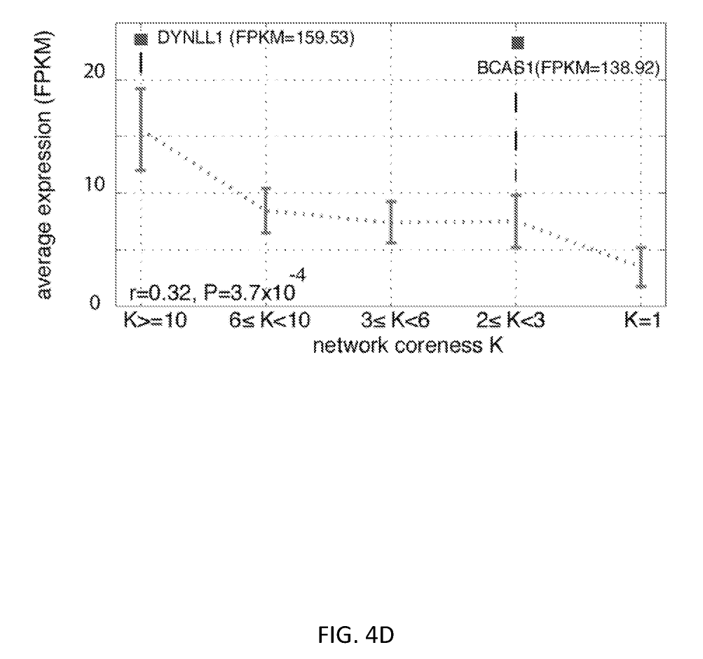

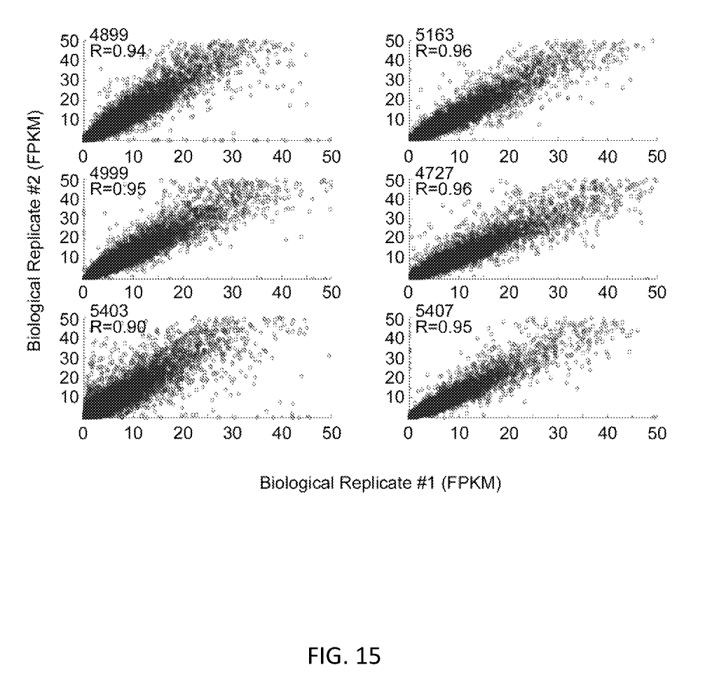

Given the apparent importance of oligodendrocytes in the corpus callosum, we further hypothesized that gene expression in this module is likely to be perturbed in the corpus callosum of ASD patients. We obtained postmortem samples from six young Caucasian males with a diagnosis of autism together with their respective matched controls from the NICHD Brain and Tissue Bank (Table 5). Total RNAs were prepared and subjected to high-coverage (180M reads/sample) deep RNA-sequencing. Biological replicates (with same sequencing depth) were performed on half of the samples, using different sections of the same tissue block. The biological replicates produced highly reproducible results with a median Pearson's coefficient equal to 0.95 (range 0.9-0.96; FIG. 15), whereas the correlations among samples from different individuals were substantially lower (median correlation coefficient 0.89, P=4.4e-3, Wilcoxon ranksum test), demonstrating the high intra-individual reproducibility of our technique. Because gene expression in the brain is age-dependent in patients with autism (Chow et al. (2012) PLoS Genet 8:e1002592), we compared gene expression in each case-control pair with identical age, ethnicity, sex, and comparable post-mortem intervals (PMIs). We then identified genes showing the most extreme expression changes in at least 1 case-control pair (fold-change >2, above the 97.5% upper bound for up-regulation and below 2.5% for down-regulation across the entire transcriptomes, Table 6). Genes encoding components of the module #13 showed significant enrichment for the differentially expressed genes relative to the genes encoding the entire protein interaction network (P=5e-4, hypergeometric test, FIG. 4A). We conducted comparisons against two control gene sets: a complete list of 1,886 known synapse-related genes (the synaptome in FIG. 4A) from SynaptomeDB (Pirooznia et al. (2012) Bioinformatics 28:897-899), and the other control included a list of known 383 autism candidate genes represented on the network. In each case, the gene set contained a similar fraction of differentially expressed genes as the entire transcriptome background (P=0.39 and 0.14, hypergeometric tests, respectively). Thus, expression of module #13, but not synaptic genes in general or known ASD candidate genes, was significantly altered in the corpus callosum of the ASD patients relative to the matched controls.

A Network View of the Candidate Loci in this ASD Module

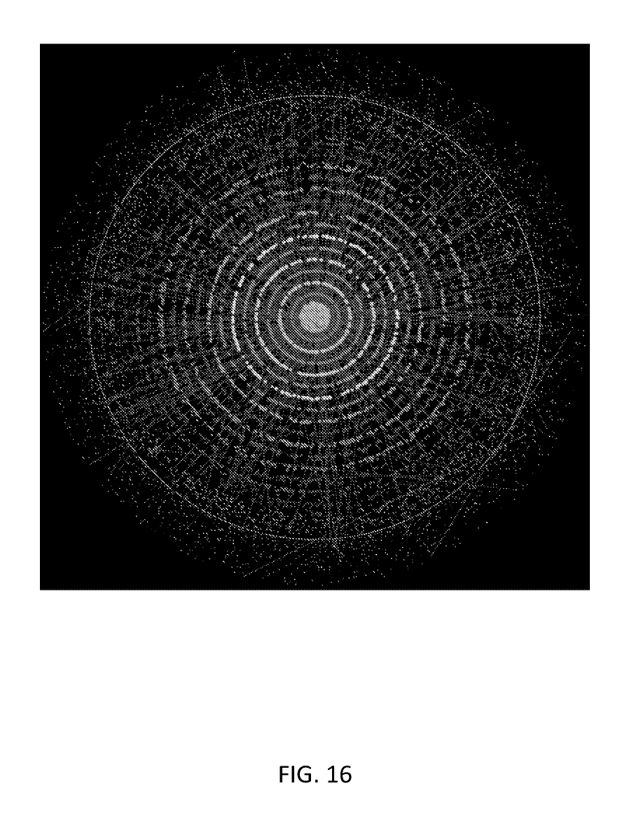

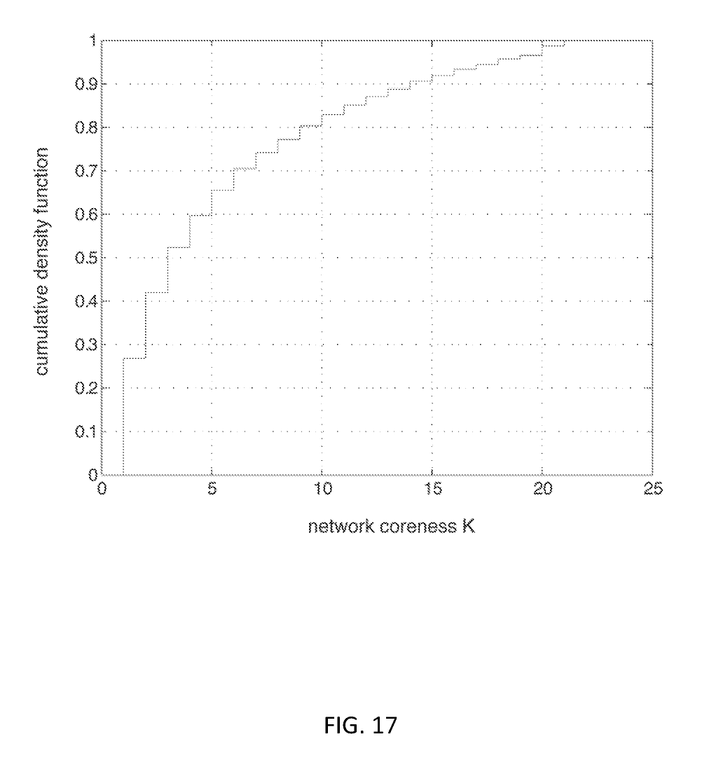

We postulated that genes associated with ASDs might show common patterns in their topological positions on the molecular network, and thus we used the protein interaction network to integrate our findings from the genome sequencing and expression analyses for the module. The global interactome can be viewed as a layered structure with proteins distributed from central cores to peripheral layers. This can be revealed by the k-core decomposition algorithm (see the layered structure in FIG. 16), where the coreness K of a protein describes its closeness towards the network center. Proteins with K=1 are peripheral nodes that are individually connected, and proteins with K≧10 lie in the center of the network (the entire K distribution for this module is shown in FIG. 17). A previous study has shown that the proportion of essential and conserved proteins increased successively towards the network's innermost cores (Wuchty & Almaas (2005) Proteomics 5:444-449).

By combining the 38 genes with at least one significant nonsynonymous variant detected from our whole-genome and exome sequencing (FIG. 1A), we found that protein products of this gene set are significantly more positioned towards the network center relative to those of the human proteome background (P=0.02, Wilcoxon ranksum test). Since this may reflect the elevated connectivity of this module as a whole on the network (P=3.6e-4, Wilcoxon ranksum test), we examined the fraction of genes with the significant variants as a function of their coreness K in the network. As shown in FIG. 4B, a significantly high proportion of central proteins in the network were affected by mutations in individuals with ASD (P=4.5e-2, hypergeometric test), whereas a significant depletion was manifested in the intermediate layer (3≦K<6) (P=0.01, hypergeometric test). The peripheral nodes also were enriched for mutations in the module but these were not statistically significant. By randomly sampling the same number of genes from the module 10,000 times, we found that the particular U-shape distribution was not expected by chance (P=4.0e-4), suggesting that network topology is indeed correlated with gene mutation frequency in ASD patients.

We also examined brain tissue gene expression as a function of network coreness K. Analysis of the different layers of the network revealed that protein products of the genes centered in the network (K≧10, FIG. 4B) were significantly biased towards the corpus callosum-specific sub-component (Group 1; FIG. 4C, P=0.01, hypergeometric test). These observations were also observed using the independent 500-patient cohort (P≦0.05, hypergeometric test). Further analysis of the corpus callosum RNA-sequencing data from the six non-autistic subjects (Table 5) revealed a positive correlation between the network coreness and their expression levels for individual genes in this synaptic module (r=0.32, P=3.7e4, FIG. 4D). These observations collectively indicate that the central genes may play fundamentally important roles in the corpus callosum as they are preferentially expressed in this tissue and pathogenic mutations of ASD patients lie in these genes. We note that two genes, DYNLL1 and BCAS1, displayed extreme expression in the corpus callosum (FIG. 4D) with FPKMs>130. Examination of their expression in the three neuronal regions (BA9, BA40 and AMY, FIG. 2B) revealed that DYNLL1 is a ubiquitously expressed gene with high expression across all the brain sections, whereas the extreme expression of BACS1 was unique only in the corpus callosum (FPKM<20 in other neuronal regions). Its specific expression in the corpus callosum was further confirmed on the microarray data from Allen Brain Atlas, suggesting a novel function of this gene in the corpus callosum.

Affected Sub-Complexes in this ASD Module

To characterize the module at higher resolution, we decomposed it into 21 sub-clusters using the algorithm. Functional coherence among genes within the same sub-complexes was observed e.g. EXOC3-6 were clustered in the fourth sub-complex, consistent with their co-complex membership by recent mass spectrometry profiling (Havugimana et al. (2012) Cell 150:1068-1081). The second sub-complex contained glutamate receptors, encompassing AMPA, kainate and NMDA families, delineating the collaborative nature of these receptor proteins. Most interestingly, many known genes implicated in ASD were also co-clustered, such as the co-clustering of NLGN1-3 with NRXN2-3, suggesting mutations on these genes are likely to perturb a common protein complex. In general, except for one sub-complex (THAP10-DYNLL2-DNAL4), all others have been affected by either mutations or mis-expression of at least one member protein, suggesting a pervasive role of this module underlying ASD etiology. Notably, the sixth and eighth sub-clusters showed significant enrichment for both the differentially expressed genes (P=0.035, hypergeometric test) and the mutated genes (P=0.036, hypergeometric test), respectively. The sixth sub-cluster revealed interaction between the DLGAP proteins (DLGAP1-4) and SHANK proteins, all of which are part of the postsynaptic scaffold. SHANK2 is particularly interesting as it is preferentially mutated and mis-expressed in the corpus callosum among patient populations in our screen. In addition, genes in the eighth sub-complex were preferentially mutated in our screen, which characterized another pathway involving the corpus callosum-specific protein LRP2. Overall, these results further delineate the substructure of the components and complexes that comprise the ASD cluster.

Discussion

Most of our knowledge today about ASD genetics has been gained from genetic association or exome-sequencing analyses of large ASD patient cohorts, which allows us to begin to observe the molecular underpinnings of this disease. However, a complete picture for this disease may require an integration of ASD genetic data from different dimensions. For example, a number of studies have analyzed genes that displayed differential expression in ASD brains (Voineagu et al, (2011) Nature 474:380-384), but aberrant mutations have not yet been identified for most of these genes. Since the retention of genetic mutations within a population is strongly driven by natural selection and population demographics(Hartl & Clark, (2007) Principles of population genetics, 4th edition, Sunderland, Mass.: Sinauer Associates), mutations in genes critical for ASD are likely to depleted by purifying selection or simply by population bottleneck, preventing the identification of ASD candidate genes only from mutational analyses. In addition, another example of a gene that would be missed by differential expression studies is LRP2, whose implication in ASD was found by the sequencing studies in this study and an earlier investigation (Ionita-Laza et al. (2012) Am. J. Hum. Genet. 90(6):1002-1013); but it did not exhibit altered expression in ASD patients. These observations strongly suggest that genetic alterations leading to ASD might occur at different levels, perturbing gene regulation or affecting gene function, and highlight the importance of building an integrative model to study ASD, where genomic data from multiple independent dimensions are incorporated to reveal the hidden architecture of this disease.

The integrative framework presented in this study is such an example to unravel the natural and physical organization of components implicated in ASD. We leveraged abundant genomic data including the human protein interactome, the transcriptome data in human and mouse brain, the MRF knockout data in mouse oligodendrocytes and also the mutation data from previous ASD sequencing projects. In addition, we also independently sequenced the genomes, exomes and transcriptomes in patients' brains to validate our observations from those publically available data or to gain new insights into this disease. Our integrative approach incorporated these genomic data of diverse dimensions, suggesting several key findings relevant to autism. First, we observed the modular structure of the human protein interactome, where genes forming a natural topological cluster tend to have shared functions. In particular module #2 (with GO enrichment for gene regulation) and #13 (with GO enrichment for synaptic transmission) showed statistically significant enrichment for ASD genes. Their enriched functional categories are consistent with earlier studies for de novo mutations associated with ASD (Ben-David & Shifman (2013) Mol. Psychiatry 18:1054-1056; O'Roak et al. (2012) Nature 485:246-250). These observations suggest convergent functional modules underlying the seemingly heterogeneous mutations associated with ASD.

Because of its high enrichment, we specifically studied module #13, and a second key finding is that this module had dichotomized spatial expression pattern across human brain: one sub-component (Group 2 genes) ubiquitously expressed and one with enhanced molecular expression in the corpus callosum (Group 1 genes). Both interact extensively with each other. We confirmed using RNA-Seq, microarrays and immunohistochemical staining that the module as a whole was expressed in the corpus callosum, a brain structure predominantly constituted by axons and oligodendrocyte cells. Up-regulation of Group 1 genes was associated with oligodendrocyte maturation from OPC cells (FIG. 3D). Considering that the expression of Group 1 genes is highly enriched in the corpus callosum, we speculate that this sub-component is likely involved in differentiating OPCs in the corpus callosum. Genes in this group include KCNJ10 (potassium inwardly-rectifying channel, subfamily J, member 10), which exhibited 10-fold up-regulation from OPCs to the matured myelinating oligodendrocytes, suggesting a strong role of this gene in oligodendrocyte development. Importantly, mutations in this gene were identified among ASD patients from our exome/genome sequencing and also in an earlier study from a different patient cohort (Sicca et al. (2011) Neurobiol. Dis. 43(1):239-247). Meanwhile aberrant mutations in this gene were also found to be associated with seizure susceptibility (Buono et al. (2004) Epilepsy Res. 58(2-3):175-183), a condition commonly comorbid with ASD. These observations support the potential role of oligodendrocytes in the development of autism. Group 2 genes, in addition their relatively high expression in the corpus callosum (FIG. 2C), showed the strongest expression in neuronal regions in brain (FIGS. 2B and 3B), explaining the high enrichment signal of synaptic genes in module #13 in our initial GO enrichment analysis. This observation supports the synaptic theory of this disease.

The corpus callosum plays a central role in mediating signal communication between the brain hemispheres through the axons extending from different cortical layers; thus appropriate myelination by the oligodendrocytes for the axons is key for the process. We further observed that conditional knockout of the myelination regulatory factor (MRF) in the matured oligodendrocyte cells significantly up-regulated Group 2 genes, which were otherwise highly expressed in neuron-rich regions. Collectively given the functions of module #13 involved in the development of oligodendrocytes, the major cell type in the corpus callosum, and thus potentially explains the reduced size of the corpus callosum that has been observed to be associated with ASD (Egaas et al. (1995) Arch Neurol. 52(8):794-801).

Two recent studies (Parikshak et al. (2013) Cell 155:1008-1021; Willsey et al. (2013) Cell 155:997-1007) have implicated the superficial cortical layer (II/III) or the deep cortical regions (layer V/VI) in ASD. Callosal projection neurons are primarily localized in the superficial layers II/III (˜80%) or deep layers V/VI (˜20%); thus our study now connected the two studies suggesting a critical role of the interhemispheric connectivity circuitry, whereby disrupting its sub-components to affect the interhemispheric signal transduction through the corpus callosum will likely to give rise to ASD phenotypes. Therefore the disease etiology should be understood at the level of the complete interhemispheric connectivity circuitry, not simply by a particular brain region or cell type. This could not only explain the enrichment in ASD-associated mutations in genes highly expressed in the constitutive parts of the circuitry (superficial or deep cortical layers in the earlier studies, or in the corpus callosum in this study), but also might provide a molecular basis for the observation from the imaging studies of the under-development of the corpus callosum among ASD patients. Importantly, different from previous research, our study illustrates the role of the oligodendrocyte cells in ASD, which myelinate and support the axons in the corpus callosum for interhemispheric signal transduction. Since current ASD research has been primarily focused on neuronal regions, future study is warranted to examine the implications of other cell types in this disease.

Two groups of genes were identified previously which displayed elevated expression in the corpus callosum, but were not significantly associated with ASD (Ben-David & Shifman (2012) PLoS Genetics 8:e1002556). The overlap between our module and these genes was restricted to two genes. Meanwhile only four of our genes overlapped with those implicated by Gilman et al. (2001) Neuron 70: 898-907), where NETBAG was used to identify the functionally associated genes affected by rare de novo CNVs in autism. Notably a more recent paper considered a sub-network implicated in ASD constituted by known ASD candidate genes and their first-degree interacting neighbors (An et al. (2014) Transl. Psychiatry 4:e394; Cristino et al. (2014) Mol. Psychiatry 19:294-301). This empirical network was large and encompassed more than 2000 genes for ASD, but ˜30% of genes in our module were not captured by their empirical network. Worthy of note, based on independent yeast-two-hybrid screens, recent studies have attempted to generate the complete interactomes for individual proteins implicated in ASD (Corominas et al. (2014) Nat. Commun. 5:3650; Sakai et al. (2011) Sci. Transl. Med. 3(86):86ra49), and thus we envision a significant expansion of our current observation when the human protein interactome is more complete.

In conclusion, by using an integrative framework we were able to examine the convergence of clinical mutations onto specific disease-related pathways. The framework provided in this work might be used to uncover functional modules for other diseases, improving their risk assessment.

Materials and Methods

Network Compilation and Operations

The human protein interaction network used in this study was downloaded from BioGrid database (rel.3.1.92) (Stark et al. (2011) Nucleic Acids Res. 39:D698-704, herein incorporated by reference), where high-quality protein interactions were collected by the curation team. We removed the isolated nodes, self-interacting edges and interactions between human and non-human proteins from the network. We analyzed a total of 13,039 proteins and 69,113 interactions. To first assess the quality of this network, we examined gene co-expression between the reported interacting proteins, which has been used previously to examine the quality of protein interactions (Yu et al. (2008) Science 322:104-110). We compared gene co-expression between the BioGrid interactome with a set of benchmarked high-confidence human interacting proteins (HINT) (Das & Yu (2012) BMC Syst. Biol. 6:92; Wang et al. (2012) Nat. Biotechnol. 30:159-164; herein incorporated by reference) and also with a set of randomly paired proteins. The expression dataset encompassing 79 human tissues and cell types (Su et al. (2002) Proc Natl Acad Sci USA 99:4465-4470, herein incorporated by reference) was used for the co-expression analysis, where probe identifies from the microarray platform were mapped onto their Entrez identifiers, and signals of multiple probes corresponding to a single Entrez gene were averaged. Pearson's pairwise correlation was then computed for protein pairs in each dataset.

Having assessed the overall quality of the network, we next topologically decomposed the global protein interaction network into a set of network modules with dense interactions within a module and sparse interactions between modules. The network decomposition algorithm was first described in a previous publication, which showed significant improvement compared with other methods (Blondel et al. (2008) J Stat Mech Theory Exp 2008:P10008, herein incorporated by reference). The modules in this study were from the first-pass partitioning of the network without further grouping small modules into larger ones. This practice gave more specific insights into module functions. The power-law distribution of the module sizes (FIG. 7A) was based on a statistic test for empirical data (Clauset et al. (2009) SIAM Rev 51:661-703, herein incorporated by reference). To test whether the modularity of the network can be observed by chance, we generated 100 randomized networks by shuffling edges of each node but maintained its degree (degree-preserving shuffling (Milo et al. (2002) Science 298:824-827, herein incorporated by reference) (FIG. 7B). We also performed Markov clustering algorithm (MCL) and affinity propagation (Vlasblom & Wodak (2009) BMC Bioinformatics 10:99, herein incorporated by reference) to divide the network, but their performance was not satisfactory, where the resulting network modularity scores Q were significantly lower than that of the algorithm used in this study. These network operations were based on FUGA (Drozdov et al. (2011) BMC Res Notes 4:462, herein incorporated by reference). Network visualization was implemented by CytoScape v2.8.3 (cytoscape.org). The layered structure of the protein interaction network was decomposed with the k-core algorithm implemented by MatlabBGL (dgleich.github.io/matlab-bgl/). Visualization of the layered structure by k-core decomposition was implemented by LaNet-vi (lanet-vi.soic.indiana.edu).

We examined GO enrichment for each of the decomposed network module to infer their biological relevance. GO annotations (excluding IEA terms) were downloaded from geneontology.org (as of September 2012). The hypergeometric test was performed to determine GO enrichment, followed by FDR correction (false discovery rate). In each of the tests, we only considered modules with more than five genes. To justify this size threshold selection, we varied the threshold from 1 to 20 genes and identified n=5 was the optimal threshold, which has balanced sensitivity and specificity (FIG. 8B). Specifically, in FIG. 8B, the dotted curve with dark gray circles showed the number of clusters with GO enrichment above a given size threshold, and the line dot curve with light gray squares was the gradients of the dotted curve at each threshold, which detected the pattern changes on the dotted curve. It is clear that the number of GO-enriched clusters decreased rapidly with the increase of the threshold when the threshold was <5 (from 200 clusters at threshold n=1 down to 85 at the threshold n=5, the number of clusters curve). This threshold-sensitive pattern was recapitulated by the rapid increase in the gradients at each threshold points, especially by the two consecutive rises in the gradients from threshold n=3 to n=4 and from n=4 to n=5 (gradients curve), transitioning from a threshold-sensitive regime into a threshold-insensitive regime. After the threshold n=5, the dotted curve gradually decreased and reached convergence after n=8, accompanied with the almost flat gradient curve (the line dot curve), which, however, suggests the threshold n≧8 would be too conservative. Thus, in this study, we used the turning point n=5 as our threshold to trade-off specificity and sensitivity. Furthermore, for module #13, we also considered the sources of the curated interactions. Module #13 consists of 119 proteins mediating 275 interactions and was derived from 109 different publications (with different PubMed IDs, on average 2.5 interactions per publication), compared with a total of 16,140 PubMed IDs for 69,113 interactions in the whole network (on average 4.28 interactions per publication). The elevated diversity of experimental sources for this module suggests that its network modularity was less likely to be biased toward a particular experimental platform.

The Enrichment of Module #13 for ASD Gene Candidates Curated from SFARI

To determine the associations of the network modules with ASD, we first considered the curated genes implicated in ASD and then generalized our comparisons to genes from unbiased genome-wide screens. We first retrieved known autism-associated genes from SFARI Gene (gene.sfari.org/autdb/). Among a total of 484 genes in the database (as of February, 2013), 383 were on the protein interaction network. Different versions of these annotated genes were also considered. In addition to using the hypergeometric test to assess the enrichment of the SFARI genes in module #13, we perform a set of permutation tests to ensure that the comparison was not biased by unequal CDS length or GC content. Briefly, we compiled a list of 10,390 genes whose CDS length (the longest RefSeq transcript, Ensembl 72) was similar with the SFARI genes (P=0.24, Wilcoxon rank-sum test). Furthermore, we also compiled a list of 14,041 genes, whose GC content in CDS was similar with the SFARI genes (P=0.58, Wilcoxon rank-sum test). We then considered the intersection between the two gene sets, totaling 7,743 genes (excluding the SFARI genes). Among this gene set with indistinguishable CDS length and GC content, we randomly sampled 383 genes, the same number with the SFARI genes, for 10,000 times (the pseudo-ASD risk genes), and we found that none of the 10,000 random simulations overlapped with module #13 more than the real SFARI gene list, giving an empirical P<1e-5. We also used genes annotated by SynaptomeDB (Pirooznia et al. (2012) Bioinformatics 28:897-899) to control for potential bias from known synaptic genes in this comparison.

The Enrichment of Module #13 for ASD Gene Candidates from Genome-Wide Screens

To determine the enrichment in module #13 for genes implicated in ASD from genome-wide screens, we compared genes in module #13 with 9,782 background genes with indistinguishable CDS length and GC content (P>0.05, Wilcoxon rank-sum test, as described above), and this set of control genes was not overlapping with module #13. For each set of ASD candidate genes (identified by CNV, exome sequencing studies, etc., Table 1), we asked whether or not the module was more enriched for these ASD candidate genes than the matched control gene sets. The exact comparisons can be found in Table 1B, where we considered ASD candidate genes affected by de novo CNVs, rare CNVs, de novo disruptive, missense and silent mutations from large collection of ASD probands. The same categories of mutations identified from non-ASD individuals or the matched unaffected siblings were also analyzed in Table 1B. The references for the data sources can be found in Tables 1A and 1B. Particularly for the de novo CNV datasets, we first considered de novo CNVs (annotated as “de novo” in their final category) identified from ASD probands from a recent publication (Pinto et al. (2014) Am J Hum Genet 94:677-694, herein incorporated by reference). In addition, de novo CNVs from two early studies were also considered (Levy et al. (2011) Neuron 70:886-897; Sanders et al. (2011) Neuron 70:863-885; herein incorporated by reference). The union and the intersection of the de novo CNV datasets from Pinto et al and those from Sanders et al. (2011) or from Levy et al. were separately tested. Genes with at least one exon affected by these de novo CNVs were considered for both ASD and non-ASD subjects. The de novo CNVs for non-ASD subjects were collected from a recent publication (Kirov et al. (2012) Mol Psychiatry 17:142-153, herein incorporated by reference). This control CNV dataset was combined with those identified from the unaffected siblings in Sanders et al. and Levy et al. Since these de novo CNVs affected thousands of genes in the genome, we also considered a small set of strong candidate genes affected by the ASD-associated high-confidence de novo CNVs in this comparison, and these genes were identified from a previous study (Noh et al. (2013) PLoS Genet 9:e1003523, herein incorporated by reference).

Collection of Genes Involved in Other Psychiatric Diseases

We additionally tested enrichment signals in module #13 for genes implicated in schizophrenia, intellectual disability and Alzheimer's diseases. Genes in schizophrenia were obtained from SZGR (bioinfo.mc.vanderbilt.edu/SZGR/index.jsp),where 38 core genes and 278 protein-coding genes representing confident loci from previous genome-wide association studies were considered. 613 genes implicated Alzheimer's disease were obtained from AlzGene (alzgene.org). Genes implicated in intellectual disability were collected in a recent publication (Parikshak et al. (2013) Cell 155:1008-1021, herein incorporated by reference).

Whole-Genome and Exome-Sequencing Protocols

Sample Information

Samples were requested from two sources, Autism Speak's Autism Tissue Program (ATP) and NICHD Brain and Tissue Bank (NICHD). Sample information can be found in Table 2. Autism diagnosis was confirmed by the clinical practitioners in the brain banks with ADI-R (Autism Diagnosis Interview-Revised). The ATP samples covered the most case DNAs in the ATP's repository (excluding 15q duplication, epilepsy, Angelman syndrome samples or samples from patients' siblings or samples with no sufficient DNA amount).

Sequencing Protocol

The genomic DNAs from ATP were extracted from the occipital lobe, Broadmann Area (BA19). We received frozen tissue blocks (postmortem corpus callosum) of six patients from NICHD and extracted genomic DNAs with the use of QIAGEN's DNeasy Blood & Tissue Kit. We used 5 lg DNAs for genome sequencing and 3 lg DNAs for exome sequencing. DNA quality was examined on agarose gel electrophoresis prior to library preparation. Sequencing was on Illumina's HiSeq 2000 platform with 101×2 pair-end adaptors. WGS samples were subject to standard Illumina's procedures with variants called by the company's software CASAVA. The called variants were further validated with the Illumina Omni genotyping SNP array with overall concordance rates of about 99.28%.

The variants were further filtered by removing variants falling in the segmental duplication, simple repeat regions, etc. For exome sequencing, GATK (ver. 2.3.9) was used to call variants by aggregating samples over the targeted intervals designed for exome capture, reaching the average ratio of Ti/Tv 3.18. Agilent SureSelectXT kit (Human All Exon V5+UTRs) was used for exome pull-down in this study. Coverage and Ti/Tv values (transition to transversion rates) for individual samples in WGS and exome sequencing can be found in Tables 3 and 4. Variants were annotated using ANNOVAR (Wang et al. (2010) Nucleic Acids Res 38:e164) based on human genome build hg19.

Analysis

Fisher's exact test was used to identify alleles overrepresented in the patient cohort. 1,000 Genome variants' allele frequencies in all samples or only in Europeans were referenced in the analysis. The P-values for variants in this module were further corrected with the Benjamini-Hochberg procedure. The functional consequences of the identified variants were tested by MutationTaster (Schwarz et al. (2010) Nat Methods 7:575-576, herein incorporated by reference), where the automatic annotations based on the 1,000 Genome frequencies were overridden by the prediction from the original Bayesian classifier. Phenotypic analysis of the identified genes was based on the component of Human-Mouse: Disease Connection in Mouse Genome Informatics (informatics.jax.org/humanDisease.shtml).

Validation Using dbGAP Data

We were approved to use one exome-sequencing dataset in dbGAP, which sequenced a larger patient population in previous study (Liu et al. (2013) PLoS Genet 9:e1003443, herein incorporated by reference). Half of the samples were sequenced in Broad Institute (by the Illumina platform) and the other half in Baylor College Medicine (BCM, by the SOLiD platform). Due to incomplete data deposited in dbGAP for those sequenced on the Illumina platform, we were only able to study the subjects sequenced by BCM, including 505 unrelated patients and 491 controls, all with European ethnicity. Variants showing the most significant deviation in their allele frequencies from the control subjects were identified with a regression analysis. We regressed case/control frequencies reciprocally, followed by a residue analysis that identified outliers exceeding the upper 5% bound of the residue distribution modeled by a t-distribution.

Expression Analyses of the Module Across Brain Sections

Expression data were from Allen Brain Atlas (Hawrylycz et al. (2012) Nature 489:391-399, herein incorporated by reference), where gene expression was measured with microarrays across hundreds of anatomical sections in two representative individuals (9,861 and 10,021). The microarray data had been normalized and post-processed by Allele Brain Atlas, and we considered 295 brain sections that were measured in both individuals (by matching the brain section identifiers). Expression of a given gene in a given tissue was then averaged over the two individuals to reduce the potential individual-specific fluctuations. In addition, signals of multiple probes mapped onto the same transcripts were also averaged in this analysis. The expression profiles were then normalized across sections followed by a hierarchical clustering, which allowed identifying gene groups sharing similar spatial expression patterns. In each brain section, the absolute expression of genes in Group 1 and 2 was also compared against the transcriptomic background in the corresponding section. Tissue specificity index was computed for individual genes across the 295 brain sections using the following formula defined in a previous study (Yanai et al. (2005) Bioinformatics 21:650-659, herein incorporated by reference), s=PN i=1 (1−xi)/N−1, where s is the tissue specificity index of a given gene, N is the total number of different brain sections, and xi is the gene's expression in a section, i. Expression breadth of a given gene was determined by the number of brain sections where the gene is active, and we varied the threshold to define gene activity based on the distribution of the absolute gene expression across the transcriptomes in the 295 brain sections (FIG. 12). The thresholds chosen in our comparison were 15, 25 and 50% of the data points across all genes, and expression values below these cutoffs were deemed to be inactive.

Genes in this module were further mapped onto the mouse genome by identifying their one-to-one mouse orthologs based on Ensembl Gene (as of August, 2013). Mouse expression data for neurons, oligodendrocytes and astrocytes were retrieved from a previous study (Cahoy et al. (2008) J Neurosci 28:264-278, herein incorporated by reference). Chi-square test was used to determine the imbalanced distribution of genes in Group 1 and 2 in the neuron and glial cluster, respectively (FIG. 3B). Mouse expression data in the oligodendrocyte precursor cells (OPCs), the mature oligodendrocytes (OLs) and the MRF conditional knockouts were retrieved from a previous study (Emery et al. (2009) Cell 138:172-185, herein incorporated by reference). We mapped the probes onto mouse gene symbols and averaged signals from multiple probes mapped onto the same genes. Expression across multiple biological replicates under the same condition was averaged.

Immunohistochemistry Analysis of the Postmortem Corpus Callosum

Immunohistochemistry analysis was performed on the corpus callosum from a patient (#5308) and a control subject (#4727). AntiLRP2 antibody was purchased from Abcam (cat#: ab76969, Abcam, Cambridge, Mass.). Immunohistochemistry labeling for LRP2 was carried out using the DAKO EnVision system (cat#: K4065, DAKO, Carpinteria, Calif.) at 1:100; slides were developed using the Dako Envision method as the manual suggested. Heat-induced antigen retrieval was performed with Decloaking Chamber (Biocare Medical, Concord, Calif.) in citrate buffer (pH 6.0). Human kidney carcinoma tissue and normal human ovary were used as positive and negative controls given the presence and absence of LRP2 (from literature) in these two tissues, respectively. In addition, IgG was also used as a control for the specificity of anti-LRP2. Cell types in the corpus callosum were independently identified and verified by a neuropathologist at Stanford.

RNA-Sequencing Protocols

Sample Information

Postmortem tissues of corpus callosum from 12 individuals were subject to RNA-sequencing in this study. Frozen tissue blocks were all provided by NICHD Brain and Tissue Bank. The samples were all European males, and case-control pairs were matched in terms of their age, sex and PMI (depends on tissue availability). All the control subjects have been optimized for comparisons and were selected by the brain bank to match the cases. The case-control pairs are listed in Table 5. We also biologically replicated our experiments on 6 out of 12 individuals by sectioning different areas of the tissue blocks. In addition to the corpus callosum, we also sequenced three brain sections (NICHD) for a control subject #5407 (Table 5), including Brodmann areas 9, 40, and also the amygdala.

Sequencing Protocols

Total RNA was extracted from flash-frozen tissue samples using Trizol reagent. Then, the total RNA was treated with RNase-Free DNase (Qiagen) followed by purification with RNeasy MinElute Cleanup Kit (Qiagen) following the manufacturer's instructions. 2 lg of total RNA each sample was subject to RNA-Seq library preparation with ScriptSeq Complete Gold Kit from Epicentre (Cat. #SCL24EP, Madison, Wis.) following the manufacturer's instructions. In brief, ribosomal RNA was depleted from total RNA using Ribo-Zero magnetic beads, and then, the ribosomal RNA-depleted RNA was purified and fragmented. Random primer tailed with Illumina adaptor was used to perform reverse transcription to get cDNA library. Adaptor sequence was added to the other end of cDNA library with a Terminal-Tagging step. The cDNA library was amplified with Illumina primers provided with this kit. The product was size selected (350-500 bp) from 2% agarose E-gels (Invitrogen) and sequenced in 1 lane per sample on Illumina's HiSeq 2000 platform.

Analysis

The sequenced 101×2 pair-end fragments were mapped against the human RefSeq transcriptome using TopHat v2.0.8 (tophat.cbcb.umd.edu). Quantitation of expression levels was computed with CuffLinks v2.0.2 (cufflinks.cbcb.umd.edu). We excluded genes with low expression in both cases and controls (FPKM<1) to avoid numerical fluctuations by small numbers and retained ˜12,000 highly expressed genes in this study (with “OK” status from Cufflinks calculation), which were likely more relevant to the physiology of this particular tissue type. We also retrieved the medical and neuropathology records of these patients and found that three patients had no documented medication history related to ASD. The other three patients took medications to correct their ASD-related behaviors; however, the potential drug targets (determined by microarray study upon drug exposure or literature curation, data not shown) were not present in our module. Therefore, medication cannot fully explain the dys-regulated genes in our module.

Human Subjects

This study was exempt from Stanford IRB review since only postmortem brain tissues from de-identified and deceased individuals were examined in this study. Brain tissues/DNA extracts were obtained from ATP and NICHD, where informed consent was obtained from all subjects. The experiments conformed to the principles set out in the WMA Declaration of Helsinki and the Department of Health and Human Services Belmont Report.

Data Availability

RNA-sequencing data are deposited in GEO with the accession identifiers: GSE62098 and GSE63513. DNA-sequencing data are deposited in SRA with the accession identifiers SRP050187.

While the preferred embodiments of the invention have been illustrated and described, it will be appreciated that various changes can be made therein without departing from the spirit and scope of the invention.

TABLE 1A
Validation for the enrichment of ASD genes in this module

| | Validation method | Dataset | Conclusion |
|---|---|---|---|
| SFARI-based | Comparing with the | synaptomeDB | Our module is more enriched |
| validation | human synaptome | SFARI | for ASD genes than that of genes |
| | background | | in the synaptome background |
| | | | (P = 3.28e−8, Fisher's exact test) |
| | Enrichment of the non- | synaptomeDB | Genes in our module not |
| | synaptic genes in | SFARI | annotated as synaptic genes |
| | module | | also showed enrichment for ASD |
| | | | genes (P = 1.64e−4, |
| | | | hypergeometric test) |
| | High-confidence loci | SFARI (gene-scoring | Significant enrichment |
| | (with syndromic | module, category S) | (P ≦ 3.85e−06, Fisher's exact) |
| | mutations) and different | | |
| | SFARI versions | | |
| Independent | Enrichment of genes | Sanders, et al; O'Roak, | Significant enrichment |
| validation | affected by de novo ASD | et al.; Neale, et al., | (P = 0.029, Fisher's exact) |
| | disruptive mutations | 2012, Nature | |
| | Enrichment of genes | Pinto, et al. 2014, | Significant enrichment (P = 0.01, |
| | affected by de novo CNVs | AJHG | Fisher's exact) |
| | in ASD probands | Sanders et al. and Levy | |
| | | et al. 2011, Neuron | |
| | Enrichment of the high- | Willsey, A. J. et al. Cell, | Significant enrichment (P = 3.8e−3, |
| | confidence ASD | 2014 | Fisher's exact) |
| | candidate genes with | | |
| | recurrent de novo | | |
| | disruptive mutations | | |
| | Enrichment of genes | Noh, H. J. et al. PLoS | Significant enrichment |
| | affected by de novo CNVs | Genet, 2013 | (P = 3.1105e−13, Fisher's exact) |
| | associated with ASD | | |
| | patients | | |
| | Enrichment of genes | Pinto, D. et al., Nature | Significant enrichment |
| | affected by rare ASD | 2010 | (P = 0.0475, Fisher's exact) |
| | CNVs | | |
| | Enrichment of rare | genome/exome- | Significant enrichment (P = 1.2e−3, |
| | nonsynonymous | sequencing in this | hypergeometric test) |
| | mutations | study | |
| | Replication on exome- | Liu, L. et al. PLoS | Significant overlap with the |
| | seq data for >500 | Genet. | candidate loci identified in our |
| | patients | | sequencing study |

1. SynaptomeDB (http://psychiatry.igm.jhmi.edu/SynaptomeDB/)
2. For the comparisons with previously published sequencing dataset, the control gene
set includes a set of 9782 genes with indistinguishable CDS length and GC content from
the genes in the module.
3. For comparisons involving ASD probands, the same comparisons on unaffected
sibling were also performed.

TABLE 1B
Enrichment test for genes with different types of mutations in ASD probands and unaffected siblings
Test for ASD candidate genes

| | | matched | Fold- | P (Fisher's | |
|---|---|---|---|---|---|
| CNV test | module | control | change | exact) | References (PMID) |
| de novo CNV (ASD-union, 2753 genes) | 19.33% | 11.27% | 1.7152 | 0.0124 | 24768552, 21658581, |
| | | | | | 21658582 |
| de novo CNV (ASD-intersection, 545 genes) | 5.04% | 2.07% | 2.435 | 0.0393 | 24768552, 21658581, |
| | | | | | 21658582 |
| HC CNV in ASD (203 genes) | 14.29% | 1.20% | 11.91 | 3.11E−13 | 23754953 |
| de novo CNV (nonASD, 557 genes) | 1.68% | 2.65% | 0.634 | 0.7725 | 21658581, 21658582, |
| | | | | | 22083728 |
| rare ASD CNV in ASD (407 genes) | 5.04% | 2.17% | 2.3226 | 0.0475 | 20531469 |
| de novo SNVs | | | | | 22495309, 22495306, |
| | | | | | 22495311 |
| de novo disruptive in proband (67 genes) | 2.52% | 0.54% | 4.67 | 0.03 | |
| de novo disruptive in siblings (8 genes) | 0.00% | 0.06% | 0 | 1 | |
| de novo missense in proband (366 genes) | 5.04% | 2.81% | 1.79 | 0.1543 | |
| de novo missense in siblings (109 genes) | 0.84% | 0.73% | 1.15 | 0.5826 | |
| de novo silent in proband (148 genes) | 0.84% | 1% | 0.84 | 1 | |
| de novo silent in siblings (52 genes) | 0 | 0.40% | 0 | 1 | |
| SFARI genes (484 genes) | 21% | 3.40% | 6.18 | 5.84E−13 | gene.sfari.org |
| HC SFARI genes (category S) | 5.04% | 0.31% | 16.26 | 3.85E−06 | HC SFARI genes (category S) |

control gene set is matched with CDS length and GC content

TABLE 2
Sample Information for DNA-sequencing

| ID | AN# | Age | Sex | PMI | Ethnicity | Diagnosis | SEQ [symbol] | Source |
|---|---|---|---|---|---|---|---|---|
| 133332 | AN03217 | 19 | M | 18.58 | European | NA | WGS | ATP |
| 133350 | AN06420 | 39 | M | 13.95 | European | ADI-R | WGS | ATP |
| 133334 | AN10833 | 22 | M | 21.47 | European | NA | WGS | ATP |
| 111305 | AN11989 | 30 | M | 16.06 | European | ADI-R | WGS | ATP |
| 133337 | AN17450 | 0 | M | 5 | European | NA | WGS | ATP |
| 111291 | AN00764 | 20 | M | 23.7 | European | Autism - confirmed by ADI- [symbol] | WGS | ATP |
| 111297 | AN19511 | 8 | M | 22.2 | European | Autism - confirmed by ADI- [symbol] | WGS | ATP |
| 111302 | AN03345 | 2 | M | 4 | European | Autism - confirmed by ADI- [symbol] | WGS | ATP |
| 133331 | AN07444 | 17 | M | 30.75 | European | NA | WGS | ATP |
| 111301 | AN09730 | 22 | M | 25 | European | Autism - confirmed by ADI- [symbol] | WGS | ATP |
| 111289 | AN16641 | 9 | M | 27 | European | ADI-R | EXOME | ATP |
| 111290 | AN00493 | 27 | M | 8.3 | European | ADI-R | EXOME | ATP |
| 111292 | AN08792 | 30 | M | 20.3 | European | ADI-R | EXOME | ATP |
| 111296 | AN08873 | 5 | | 25.5 | European | ADI-R | EXOME | ATP |
| 111299 | AN01570 | 18 | F | 6.75 | European | ADI-R | EXOME | ATP |
| 111304 | AN12457 | 29 | | 17.83 | European | ADI-R | EXOME | ATP |
| 111310 | AN08166 | 28 | | 43.25 | European | ADI-R | EXOME | ATP |
| 111313 | AN17678 | 11 | M | — | European | ADI-R | EXOME | ATP |
| 111316 | AN09714 | 60 | M | 26.5 | European | Autism - confirmed by ADI- [symbol] | EXOME | ATP |
| 111317 | AN17254 | 51 | M | 22.16 | European | ADI-R | EXOME | ATP |
| 133328 | HSB- | 8 | M | 13.8 | European | Autism - supported by | EXOME | ATP |
| | 4640 | | | | | records | | |
| 133341 | AN16115 | 11 | | 12.88 | European | ADI-R | EXOME | ATP |
| 133344 | AN08043 | 52 | F | 39.15 | European | ADI-R | EXOME | ATP |
| 133346 | AN02456 | 4 | | 17.02 | European | NA | EXOME | ATP |
| 5403 | # | 16 | | 35 | European | ADI-R | EXOME | NICHD |
| 5144 | # | 7 | M | 3 | European | ADI-R | EXOME | NICHD |
| 5308 | # | 4 | M | 21 | European | ADI-R | EXOME | NICHD |
| 5302 | # | 16 | M | 20 | European | ADI-R | EXOME | NICHD |
| 4899 | # | 14 | M | 9 | European | ADI-R | EXOME | NICHD |
| 4999 | # | 20 | M | 14 | European | ADI-R | EXOME | NICHD |

Notes -
ADI-R: autism diagnostic interview, revised;
NA: control subjects with no diagnosed autism;
WGS: whole-genome sequencing;
Exome: exome sequencing;
ATP: Autism Tissue Program;
NICHD: NICHD Brain and Tissue Bank;
PMI: postmortem interval. [symbol]
indicates data missing or illegible when filed

TABLE 3
Information for whole-genome sequencing

| | ID | | | | | | | | | |
|---|---|---|---|---|---|---|---|---|---|---|
| | 133350 | 111297 | 133334 | 133337 | 111302 | 111301 | 133331 | 111291 | 133332 | 111305 |
| Ti/Tv | 2.027 | 2.025 | 2.030 | 2.030 | 2.032 | 2.026 | 2.031 | 2.026 | 2.13 | 2.10 |
| Cvg* | 38.3 | 38.7 | 38.6 | 34.7 | 38.4 | 35.9 | 37.2 | 40.4 | 36.2 | 41.9 |
| Array* | 99.28% | 99.28% | 99.27% | 99.25% | 99.26% | 99.26% | 99.28% | 99.28% | — | — |

TABLE 4
Ti/Tv ratio and mean coverage for exome-sequencing

| | ID | | | | | | | | | | | |
|---|---|---|---|---|---|---|---|---|---|---|---|---|
| | 111289 | 111290 | 111292 | 111296 | 111299 | 111304 | 111310 | 111311 | 111313 | 111316 | 111317 | 133328 |
| Ti/Tv | 3.27 | 3.18 | 3.15 | 3.19 | 3.18 | 3.13 | 3.17 | 3.18 | 3.21 | 3.16 | 3.19 | 3.18 |
| Cvg | 107.95 | 110.68 | 115.59 | 120.55 | 113.75 | 111.03 | 202.3* | 106.51 | 125.08 | 108.86 | 123.45 | 103.55 |

| | ID | | | | | | | | |
|---|---|---|---|---|---|---|---|---|---|
| | 133341 | 133344 | 133346 | 4899 | 4999 | 5144 | 5302 | 5308 | 5403 |
| Ti/Tv | 3.19 | 3.18 | 3.19 | 3.14 | 3.16 | 3.14 | 3.22 | 3.1 | 3.22 |
| Cvg | 130.47 | 97.07 | 120.41 | 117.97 | 113.14 | 120.11 | 124.26 | 112.57 | 127.27 |

*Every 2 samples were sequenced in one HiSeq lane, and the sample 111310 were sequenced alone in one lane, which doubled its coverage. Cvg is the mean coverage for each sample. Array is the percentage of agreement with genotyping validation with OminChip.

TABLE 5
Sample Information for RNA-sequencing

| | | | | | Ctl | | | | |
|---|---|---|---|---|---|---|---|---|---|
| Case ID | Age | Sex | PMI | Ethnicity | ID | Age | Sex | PMI | Ethnicity |
| 5403 | 16 | M | 35 | European | 5407 | 16 | M | 33 | European |
| 5144 | 7 | M | 3 | European | 5391 | 7 | M | 12 | European |
| 5308 | 4 | M | 21 | European | 4670 | 4 | M | 17 | European |
| 5302 | 16 | M | 20 | European | 5242 | 15 | M | 9 | European |
| 4899 | 14 | M | 9 | European | 5163 | 14 | M | 12 | European |
| 4999 | 20 | M | 14 | European | 4727 | 20 | M | 5 | European |

TABLE 6
Genes showing extreme expression difference (FPKM) in at least one matched case-control pair(s)

| | case- | ctl- | case- | ctl- | case- | | case- | ctl- |
|---|---|---|---|---|---|---|---|---|
| | 4899 | 5163 | 4999 | 4727 | 5144 | ctl-5391 | 5302 | 5242 |
| Symbols | AGE-14 | AGE-14 | AGE-20 | AGE-20 | AGE-7 | AGE-7 | AGE-16 | AGE-15 |
| ACTN2 | 8.17378 | 6.91817 | 7.05457 | 7.6857 | 4.85415 | 12.3486 | 7.46995 | 13.5616 |
| ATP2B2 | 10.4157 | 10.2315 | 14.496 | 2.27459 | 4.07595 | 7.84837 | 1.95087 | 1.29592 |
| BCAS1 | 54.125 | 59.9034 | 57.4663 | 80.9105 | 45.298 | 63.5851 | 77.5107 | 97.8034 |
| CAMK2A | 12.4896 | 17.5579 | 24.9935 | 3.8031 | 10.5568 | 11.6632 | 1.94691 | 2.23104 |
| CNTNAP4 | 34.1099 | 37.2074 | 52.8401 | 65.2879 | 27.0515 | 35.0873 | 18.6162 | 81.2807 |
| DGKZ | 6.60179 | 4.92138 | 7.28702 | 2.62397 | 3.08711 | 7.22943 | 2.03579 | 2.42599 |
| DLGAP2 | 0.88074 | 0.85067 | 0.86657 | 0.31541 | 0.466513 | 0.964308 | 0.157345 | 0.1543 |
| DLGAP3 | 1.79048 | 1.13156 | 0.7079 | 0.26221 | 0.446388 | 1.09454 | 0.274824 | 0.11764 |
| DYNLL1 | 76.9804 | 100.479 | 114.349 | 147.13 | 112.733 | 75.688 | 18.2117 | 133.366 |
| GDA | 5.42175 | 4.82084 | 10.0478 | 2.45284 | 4.35218 | 9.22799 | 0.365664 | 1.55957 |
| GRIA1 | 6.5753 | 5.24132 | 6.42643 | 3.39868 | 4.6167 | 5.57764 | 0.754103 | 2.04652 |
| GRIK3 | 1.55601 | 0.80589 | 1.57387 | 0.92275 | 0.631652 | 2.19164 | 0.158799 | 0.79529 |
| GRIN2A | 5.43702 | 5.23169 | 12.1565 | 3.92986 | 3.49946 | 5.99765 | 1.41466 | 2.73144 |
| GRIN2B | 6.06032 | 3.73538 | 5.5023 | 1.38787 | 2.89003 | 4.81559 | 0.350852 | 1.0746 |
| HTR2C | 2.48281 | 19.0262 | 0.76687 | 0.64221 | 0.352887 | 0.363268 | 0.381782 | 0.62936 |
| KCNA4 | 1.37102 | 0.51685 | 0.78718 | 0.26573 | 0.439702 | 0.572336 | 0.066066 | 0.07808 |
| KCNJ2 | 4.97208 | 11.6348 | 13.5834 | 18.9721 | 19.7206 | 17.288 | 7.30111 | 24.2115 |
| KCNJ4 | 1.07885 | 0.8044 | 1.28626 | 0.25274 | 0.396522 | 0.785802 | 0.261297 | 0.15163 |
| LDB3 | 2.6986 | 6.11025 | 3.73788 | 5.926 | 2.29005 | 8.6733 | 4.31096 | 8.16793 |
| LPL | 7.36867 | 1.50055 | 1.39253 | 1.42135 | 2.63802 | 0.621972 | 1.45769 | 1.32159 |
| NRXN2 | 4.87073 | 5.95951 | 4.89019 | 4.46779 | 3.49419 | 9.54515 | 3.97993 | 5.68091 |
| PGMS | 0.47905 | 1.85635 | 2.03931 | 2.67337 | 1.90154 | 1.25966 | 0.755508 | 1.98993 |
| PTPRN | 3.47961 | 2.13605 | 2.92521 | 0.49141 | 1.04898 | 2.90347 | 0.181454 | 0.30846 |
| S100A3 | 2.56913 | 0.11203 | 0.06079 | 0.08138 | 0.076386 | 0.03406 | 0.178854 | 0.25742 |
| SCN1A | 5.38941 | 6.30019 | 15.8115 | 2.74602 | 3.16211 | 3.092 | 1.8294 | 2.09287 |
| SHANK2 | 1.66051 | 1.73733 | 1.18268 | 0.63088 | 1.05491 | 1.45654 | 0.609728 | 0.37609 |
| SHANK3 | 1.24115 | 1.35859 | 0.59471 | 0.44926 | 0.537711 | 1.45191 | 1.10618 | 0.41388 |
| TBR1 | 1.46978 | 1.27778 | 2.82477 | 0.37308 | 0.989652 | 2.2465 | 0.218025 | 0.12924 |
| TJAP1 | 2.31436 | 4.58465 | 3.07065 | 4.04476 | 2.77386 | 6.35728 | 2.97949 | 5.82153 |
| ZDHHC23 | 1.39532 | 1.2062 | 3.06164 | 0.4455 | 0.987482 | 1.37944 | 0.45738 | 0.2826 |

| | case- | ctl- | case- | ctl- | |
|---|---|---|---|---|---|
| | 5308 | 4670 | 5403 | 5407 | |
| Symbols | AGE-4 | AGE-4 | AGE-16 | AGE-16 | Case-Ctl pairs showing diff. expression |
| ACTN2 | 7.54513 | 9.69953 | 10.2076 | 6.55199 | 5144_5391 |
| ATP2B2 | 0.957262 | 12.9955 | 2.83299 | 6.72334 | 4999_4727 5308_4670 |
| BCAS1 | 100.546 | 49.4591 | 83.5928 | 62.3803 | 5308_4670 |
| CAMK2A | 1.62797 | 29.614 | 1.83633 | 6.98267 | 4999_4727 5308_4670 5403_5407 |
| CNTNAP4 | 68.3625 | 29.7576 | 44.5378 | 29.4967 | 5308_4670 |
| DGKZ | 2.02202 | 14.8028 | 1.94708 | 3.19701 | 5144_5391 |
| DLGAP2 | 0.079893 | 1.66111 | 0.085811 | 0.351 | 5308_4670 |
| DLGAP3 | 0.132243 | 1.7777 | 0.168847 | 0.8147 | 5144_5391 5308_4670 |
| DYNLL1 | 95.5138 | 100.619 | 67.9696 | 44.878 | 5302_5242 |
| GDA | 0.852387 | 24.4891 | 1.01248 | 1.69001 | 4999_4727 5308_4670 |
| GRIA1 | 1.14055 | 14.8272 | 2.1878 | 2.13048 | 5308_4670 |
| GRIK3 | 0.647623 | 3.31867 | 0.60924 | 0.89717 | 5144_5391 |
| GRIN2A | 0.994802 | 13.798 | 3.74731 | 3.22638 | 5308_4670 |
| GRIN2B | 0.910705 | 13.5725 | 0.71534 | 1.40033 | 5308_4670 |
| HTR2C | 0.117263 | 1.48519 | 22.7309 | 31.4639 | 4899_5163 5308_4670 |
| KCNA4 | 0.054512 | 1.17392 | 0.144732 | 0.31821 | 5308_4670 |
| KCNJ2 | 21.5645 | 11.8395 | 11.7479 | 5.37666 | 4899_5163 |
| KCNJ4 | 0.119609 | 2.2375 | 0.120816 | 0.29597 | 4999_4727 5308_4670 |
| LDB3 | 8.58386 | 4.77803 | 7.49784 | 4.55085 | 5144_5391 |
| LPL | 0.988848 | 0.84403 | 1.51843 | 2.22148 | 4899_5163 |
| NRXN2 | 6.32916 | 9.73752 | 5.83865 | 5.11209 | 5144_5391 |
| PGMS | 1.87654 | 1.78705 | 1.34142 | 0.3585 | 4899_5163 5403_5407 |
| PTPRN | 0.251279 | 6.38684 | 0.303596 | 0.94505 | 4999_4727 5144_5391 5308_4670 |
| S100A3 | 0.050422 | 0.04706 | 0.137539 | 1.23705 | 4899_5163 5403_5407 |
| SCN1A | 2.05957 | 5.82705 | 2.78413 | 2.50353 | 4999_4727 |
| SHANK2 | 0.269225 | 3.5049 | 1.36204 | 2.77783 | 5308_4670 |
| SHANK3 | 0.473776 | 1.51758 | 0.684206 | 1.44919 | 5144_5391 5302_5242 |
| TBR1 | 0.108868 | 2.84041 | 0.191095 | 0.39978 | 4999_4727 5144_5391 5308_4670 |
| TJAP1 | 5.31939 | 3.97288 | 4.89807 | 3.74644 | 5144_5391 |
| ZDHHC23 | 0.366755 | 2.76498 | 0.825861 | 1.17759 | 4999_4727 |

1. A method of screening a subject for genetic markers associated with autism spectrum disorders (ASD) and treating the subject for the ASD, the method comprising:
a) collecting a biological sample from the subject;
b) analyzing the biological sample to determine whether a gene selected from the group consisting of GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprises a mutation associated with the ASD; and
c) treating the subject for the ASD with behavior training, occupational therapy, or special education courses if the subject has at least one mutation associated with the ASD.

2. The method of claim 1, further comprising determining which allele is present at a single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894, wherein the presence of a mutation at the single nucleotide polymorphism indicates that the subject has ASD.

3. The method of claim 1, further comprising determining which allele is present at a single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266, wherein the presence of a mutation at the single nucleotide polymorphism indicates that the subject has ASD.

4. The method of claim 1 comprising analyzing the biological sample to determine whether the genes GRIN2B, SHANK2, TBR1, DLGAP2, SYNGAP1, LRP2, NLGN3, SHANK3, and ERBB2IP comprise a mutation associated with ASD.

5. The method of claim 4, further comprising analyzing the biological sample to determine whether a gene selected from the group consisting of ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprises a mutation associated with ASD.

6. The method of claim 1 comprising analyzing the biological sample to determine whether a gene selected from the group consisting of ACTN4, ANKS1B, BCAS1, DGKZ, DLG1, DLGAP1, ERBB2IP, EXOC3, EXOC5, EXOC6, GDA, GRID2IP, GRIK3, IL16, KCNJ12, KCNJ15, KIF13B, LPL, LRP2BP, MAPK12, MPP6, MYOZ1, NOS1, SCN5A, THAP8, TNN, and UTRN comprises a mutation associated with ASD.

7. (canceled)

8. The method of claim 6 comprising analyzing the biological sample to determine whether a gene selected from the group consisting of ANKS1B, DLG1, ERBB2IP, GRID2IP, GRIK3, KCNJ12, KCNJ15, NOS1, SCN5A, UTRN comprises a mutation associated with ASD.

9. (canceled)

10. The method of claim 6, further comprising analyzing the biological sample to determine whether a gene selected from the group consisting of CNTNAP4, DLG4, DMD, GRIK2, INPP1, KIF13B, KCNJ10, LRP2, NLGN3, NLGN4X, SCN1A, and SHANK2 comprises a mutation associated with ASD.

11. The method of claim 6, further comprising analyzing the biological sample to determine whether a gene selected from the group consisting of GRIN2B, SHANK2, TBR1, DLGAP2, SYNGAP1, LRP2, NLGN3, SHANK3, and ERBB2IP comprises a mutation associated with ASD.

12. The method of claim 6, further comprising determining which allele is present at a single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894, wherein the presence of a mutation indicates that the subject has ASD.

13. The method of claim 6, further comprising determining which allele is present at a single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266, wherein the presence of a mutation indicates that the subject has ASD.

14. The method of claim 1, further comprising screening the subject for copy number variation in at least one gene selected from the group consisting of CAMK2B, DLG1, DLG4, DLGAP2, DLGAP3, DLGAP4, DYNLL1, EXOC3, KCND2, MAPK12, NLGN2, NLGN3, NLGN4X, NOS1, SHANK2, SNTA1, and SYNGAP1, wherein detection of copy number variation in at least one gene indicates that the subject has ASD.

15. The method of claim 14 comprising screening the subject for copy number variation in the genes SHANK2, DLGAP2, and SYNGAP1.

16. The method of claim 1, wherein the biological sample is blood, serum, plasma, saliva, amniotic fluid, or tissue.

17-18. (canceled)

19. The method of claim 1, wherein the subject is a developmentally disabled child.

20. The method of claim 19, further comprising providing early behavior training for the child if the child has at least one genetic marker associated with ASD.

21. The method of claim 1, wherein the subject has a sibling who has ASD, a parent who has ASD, or is a parent or relative of a developmentally disabled child.

22-23. (canceled)

24. The method of claim 1, wherein at least one mutation is a single nucleotide polymorphism.

25. The method of claim 1, wherein at least one mutation comprises a substitution, an insertion, a deletion, or a rearrangement.

26. The method of claim 1, wherein at least one mutation comprises a missense mutation, a nonsense mutation, a frameshift mutation, a splice-site mutation, an inversion, or a translocation.

27. A method of determining risk of a human offspring developing an autism spectrum disorder (ASD) and treating the offspring for the ASD, the method comprising detecting in a biological sample from the mother or potential mother of the offspring at least one mutation associated with the ASD in a gene selected from the group consisting of GRIN2B, SHANK2, TBR1, DLGAP2, SYNGAP1, LRP2, NLGN3, SHANK3, ERBB2IP, ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN, wherein the presence of at least one mutation indicates an increased risk of the offspring developing the ASD; and providing early behavior training to the offspring if the offspring has at least one mutation indicating the offspring has increased risk of developing the ASD.

28-29. (canceled)

30. The method of claim 27 comprising analyzing the biological sample to determine whether the genes GRIN2B, SHANK2, TBR1, DLGAP2, SYNGAP1, LRP2, NLGN3, SHANK3, and ERBB2IP comprise a mutation associated with ASD.

31. The method of claim 30, further comprising analyzing the biological sample to determine whether a gene selected from the group consisting of ACTN4, ANKS1B, BCAS1, CNTNAP4, DGKZ, DLG1, DLG4, DLGAP1, DMD, ERBB2IP, EXOC3, EXOC5, EXOC6, GDA, GRID2IP, GRIK2, GRIK3, IL16, INPP1, KIF13B, KCNJ10, KCNJ12, KCNJ15, LPL, LRP2, LRP2BP, MAPK12, MPP6, MYOZ1, NLGN3, NLGN4X, NOS1, SCN1A, SCN5A, SHANK2, THAP8, TNN, and UTRN comprises a mutation associated with ASD.

32. The method of claim 27 comprising analyzing the biological sample to determine whether a gene selected from the group consisting of ACTN4, ANKS1B, BCAS1, DGKZ, DLG1, DLGAP1, ERBB2IP, EXOC3, EXOC5, EXOC6, GDA, GRID2IP, GRIK3, IL16, KCNJ12, KCNJ15, KIF13B, LPL, LRP2BP, MAPK12, MPP6, MYOZ1, NOS1, SCN5A, THAP8, TNN, and UTRN comprises a mutation associated with ASD.

33. (canceled)

34. The method of claim 32 comprising analyzing the biological sample to determine whether a gene selected from the group consisting of ANKS1B, DLG1, ERBB2IP, GRID2IP, GRIK3, KCNJ12, KCNJ15, NOS1, SCN5A, UTRN comprises a mutation associated with ASD.

35. (canceled)

36. The method of claim 32, further comprising analyzing the biological sample to determine whether a gene selected from the group consisting of CNTNAP4, DLG4, DMD, GRIK2, INPP1, KIF13B, KCNJ10, LRP2, NLGN3, NLGN4X, SCN1A, and SHANK2 comprises a mutation associated with ASD.

37. The method of claim 32, further comprising analyzing the biological sample to determine whether a gene selected from the group consisting of GRIN2B, SHANK2, TBR1, DLGAP2, SYNGAP1, LRP2, NLGN3, SHANK3, and ERBB2IP comprises a mutation associated with ASD.

38. The method of claim 32, further comprising determining which allele is present at a single nucleotide polymorphism selected from the group consisting of rs114460450, rs4072111, rs1801177, rs114842875, rs11068428, rs17526980, rs3213837, rs34355135, rs75029097, rs117927165, rs41315493, rs147232488, rs201998040, rs144800425, rs3213760, rs138457635, rs34693334, rs41311117, rs35430440, rs200424265, rs188319299, rs61752956, rs149249492, rs199777795, rs147877589, rs77436242, rs200240398, rs202120564, rs2917720, rs149484544, rs143174736, rs148359556, rs145307351, rs72468667, and rs144914894, wherein the presence of a mutation indicates that the subject has ASD.

39. The method of claim 32, further comprising determining which allele is present at a single nucleotide polymorphism at a chromosome position selected from the group consisting of chr14:57700582, chr2:191224928, chr5:453976, chr6:144803462, chr10:75394301, chr2:170013962, chr5:462089, chr1:37346322, chr1:175106036, chr2:166856252, chr2:170060566, chr6:102513700, chr7:6537431, chr8:28929209, chr8:28974353, chr11:70336414, chr12:99548216, chr16:76587326, chr16:76587326, chr19:36530613, chr20:52583542, chr20:52601885, and chr21:39671266, wherein the presence of a mutation indicates that the subject has ASD.

40. The method of claim 27, further comprising analyzing the biological sample to determine whether at least one gene selected from the group consisting of CAMK2B, DLG1, DLG4, DLGAP2, DLGAP3, DLGAP4, DYNLL1, EXOC3, KCND2, MAPK12, NLGN2, NLGN3, NLGN4X, NOS1, SHANK2, SNTA1, and SYNGAP1 shows copy number variation, wherein detection of copy number variation in at least one gene indicates that the subject has ASD.

41. The method of claim 40 comprising analyzing the biological sample to determine whether the genes SHANK2, DLGAP2, and SYNGAP1 show copy number variation.

42. The method of claim 27, wherein the offspring is a neonate or a fetus.

43. (canceled)

44. The method of claim 27, wherein said biological sample is obtained prior to conception and said detecting occurs prior to conception.

45. The method of claim 27, wherein the mother or potential mother has a child with an ASD or has a familial history of ASD.

46. (canceled)

47. The method of claim 27, wherein the biological sample is selected from the group consisting of amniotic fluid, placental tissue, blood, serum, and plasma.

48-66. (canceled)

67. A method for diagnosing and treating an autism spectrum disorder (ASD) in a subject, the method comprising:
a) measuring the level of one or more biomarkers in a biological sample derived from the subject, wherein the one or more biomarkers comprise one or more polynucleotides comprising nucleotide sequences from genes or RNA transcripts of genes, including but not limited to, ACTN2, ATP2B2, BCAS1, CAMK2A, CNTNAP4, DGKZ, DLGAP2, DLGAP3, DYNLL1, GDA, GRIA1, GRIK3, GRIN2A, GRIN2B, HTR2C, KCNA4, KCNJ2, KCNJ4, LDB3, LPL, NRXN2, PGM5, PTPRN, S100A3, SCN1A, SHANK2, SHANK3, TBR1, TJAP1, and ZDHHC23, and gene products thereof;
b) analyzing the levels of the biomarkers in conjunction with respective reference value ranges for said plurality of biomarkers, wherein differential expression of one or more biomarkers in the biological sample compared to one or more biomarkers in a control sample from a normal subject indicates that the subject has the ASD; and
c) treating the subject for the ASD with behavior training, occupational therapy, or special education courses if the subject is diagnosed as having the ASD.

68. (canceled)

69. The method of claim 67, wherein the biological sample is blood, serum, plasma, saliva, amniotic fluid, or tissue.

70-100. (canceled)
